(19) United States
(12) Patent Application Publication
(10) Pub. No.: US 2022/0330541 A1
(43) Pub. Date: Oct. 20, 2022

(54) Methods for the Preservation of Reagent Red Blood Cells Using Carbon Monoxide

(71) Applicant: Hemanext Inc., Lexington, MA (US)

(72) Inventors: Tatsuro YOSHIDA, West Newton, MA (US); Andrew Dunham, Tower Lakes, IL (US)

(73) Assignee: Hemanext Inc., Lexington, MA (US)

(21) Appl. No.: 17/639,823

(22) PCT Filed: Sep. 4, 2020
(86) PCT No.: PCT/US2020/049510

§ 371 Date: Mar. 2, 2022

Related U.S. Application Data
(60) Provisional application No. 62/896,360, filed on Sep. 5, 2019.

(51) Int. Cl. A01N 1/02; A61K 35/18

(52) U.S. Cl. CPC A01N 1/0205; A61K 35/18

(57) ABSTRACT

This application provides methods, compositions, and kits for use blood group determination and the preparation of improved red blood cell containing reagents for use in blood typing of blood prior to its use in transfusion medicine.

### CROSS-REFERENCE TO RELATED APPLICATION

This application claims the benefit of U.S. Provisional Application No. 62/896,360, filed Sep. 5, 2019, which is incorporated by reference in its entirety herein.

### FIELD OF THE INVENTION

This invention relates to the field of blood group determination and the preparation of improved red blood cell containing reagents for use in blood typing of blood prior to its use in transfusion medicine. This invention also relates to improved storage and in vivo circulation of red blood cells for drug delivery.

### BACKGROUND OF THE INVENTION

Red Blood Cell (RBC) reagents are manufactured from blood collected from established donors with well-characterized phenotype. RBCs are diluted and packaged in diluent solution to be used as standards for determining blood type of processed RBCs at blood centers/blood banks. In most cases, automated devices are employed for the blood type determinations and for characterizing other important RBC quality parameters. To preserve the surface antigens, RBCs are not fixed and are therefore labile due to the accumulation of storage lesions. These time dependent changes limit the shelf life of these important RBC preparations to about 9 weeks (63 days).

RBC evolved to provide transport oxygen throughout the body, where except for the surface, diffusion from ambient air cannot be relied. RBCs accomplish this function by packing very high concentrations of hemoglobin containing oxidizable ferrous iron in the cytosol. During circulation for approximately 120 days, RBCs are maintained to transport oxygen by elaborate network of metabolic/redox enzymes. However, these elaborate evolutional optimizations are no longer operable once RBCs are removed from circulation and stored hypothermically, resulting in storage-induced damage (storage lesions) that accumulates over the shelf life of stored RBC. One of two major driving forces for development of storage lesions is oxidative damage that was known but not addressed systemically until recently.

Chemical oxidation of iron in hemoglobin is the central reaction that initiates oxidative stress in stored RBCs, the major element for the development of the storage lesion. RBCs contain high concentrations of reactive ferrous iron in the prosthetic group of hemoglobin together with a high concentration of dissolved oxygen. Four iron moieties (ferrous state) in hemoglobin react chemically with oxygen to form methemoglobin (ferric state). As a byproduct, superoxide anion is generated, which is converted by superoxide dismutase to form H₂O₂, a major reactive oxygen species (ROS) and a substrate for hydroxyl radical (OH.) generation. In vivo, methemoglobin is reduced back to hemoglobin by reductase enzymes but these enzyme activities are curtailed under hypothermic storage conditions. Coupled with higher dissolved oxygen concentrations stemming from increased solubility at low temperature, this phenomenon results in enhanced production of methemoglobin and superoxide anion. ROS molecules react with lipids and structural proteins in RBC damaging integrity and reducing in vivo circulation life. ROS also attack critical enzymes or surface molecules that makes ‘product’ RBCs valuable, diminishing efficacy or utility.

Hemoglobin's affinity toward CO is about 400 times higher than O₂, and CO does not readily react chemically with heme. The heme-CO complex is very stable, and thus greatly reduces oxidative RBC storage lesions as hemoglobin oxidation by oxygen is the main driver of oxidative stress during hypothermic RBC storage. Unlike O₂, CO does not react readily react with ferrous iron however it prevents Hb oxidation by stabilizing it as Hb-CO, and greatly reduces oxidative storage lesion development in hypothermically (i.e., 1-6° C.) stored RBCs. ROS damage can be further reduced by storing the RBCs under oxygen free conditions, either under CO or an inert gas like nitrogen.

Although high affinity binding of CO to Hb renders Hb and RBCs containing Hb-CO useless as an oxygen carrier until the CO is released, the stabilization of Hb by CO is beneficial during storage and can extend the shelf life of the Reagent RBCs not only prior to being opened or reconstituted for use, but also after opening. Much more than RBCs for transfusions, the Reagent RBCs are highly characterized and carefully controlled reagents of great value. Even small improvements to the shelf life can significantly reduce costs for blood banking operations. CO-treatment of reagent RBCs reduces storage lesion accumulation and prolongs the shelf life.

In addition to the ability of red blood cells to transport oxygen, red blood cells are also utilized as biocompatible carriers for different drugs, peptide molecules, and enzymes. Red blood cells can be engineered to treat various diseases through the expression of biotherapeutic proteins on the cell surface or within the cytosol. These red blood cells are utilized to treat cancer, autoimmune diseases, gastrointestinal diseases, and to replace missing enzymes in patients with enzyme deficiencies. An extensive review of cells for drug delivery is provided in Shi, J., et al., “Engineered red blood cells as carriers for systemic delivery of a wide array of functional probes,” PNAS 111(28): 10131-10136 (2014), Deshmukhe A and Shetty S, “Resealed erythrocytes: a novel and promising drug carrier,” Int J Pharm Sci Res 8(8):3242-51 (2017), Hamadi and Tajerzadeh, “Carrier Erythrocytes: An Overview,” Drug Delivery 10: 9-20 (2003); Harisa et al., “Application and safety of erythrocytes as a novel drug delivery system,” Asian Journal of Biochem. 6(4): 309-321 (2011), Ravilla et al., “Erythrocytes as Carrier for Drugs, Enzymes, and Peptides,” Journ. of Applied Pharm. Sci. 2(04): 166-176 (2012), Sprandel and Zöllner, “Osmotic fragility of drug carrier erythrocytes,” Res. Exp. Med. 185: 77 (1985), US Patent Publication No. 2017/0020926, US Patent Publication No. 2018/0085402, US Patent Publication No. 2018/0153989, US Patent Publication No. 2018/0135012, and U.S. Pat. No. 9,644,180, each of which is incorporated by reference in its entirety.

One issue with the red blood cells for drug delivery arises during storage. Oxidative damage initiates red blood cell storage lesions in conventionally stored red blood cells with or without pharmaceutical agents for drug delivery. In addition, non-oxidative damage also reduces the circulation life of drug delivering RBCs. When delivering therapeutic agents, maintaining high quality during storage and ensuring consistent circulation life are critical. Given the dose dependency of active agents, methods to ensure predictable and consistent quality of RBCs for drug delivery are needed. In contrast to RBCs for transfusion, the ability of the RBCs for drug delivery to deliver oxygen is of secondary importance. Rather, the reduction of lesions and stabilization of the cells for long term circulation are the primary goals. Thus, for pharmaceutical agent delivery, further methods are needed to reduce oxidative damage as well as stabilize red blood cells to extend storage shelf-life and circulation life within the body of a treated patient.

Here we provide for the first time a storage method for improving the shelf-life of red blood cells used for drug delivery and increased circulation time of red blood cells by both reducing oxidative damage and stabilizing hemoglobin. This is accomplished by reducing oxygen levels and preparing stabilized derivatives of hemoglobin during storage. In one aspect, carbon monoxide is added to the hemoglobin containing cells to form stable carboxyhemoglobin. As provided above, hemoglobin affinity to carbon monoxide is approximately 400 times that of oxygen, and carbon monoxide does not readily chemically react with ferrous iron of hemoglobin. Here, we also provide for several alternatives to carbon monoxide, including cyanide and azide. Cyanide reacts with oxidized hemoglobin (methemoglobin) to form the very stable cyano-methemoglobin. This complex does not readily degrade. The cyano-methemoglobin complex can be formed by numerous oxidizing agents such as methylene blue, phenylmehtylsulfate, and potassium ferricyanide. Azide also reacts with methemoglobin to form the stable azido-hemoglobin complex.

### SUMMARY OF THE INVENTION

The present disclosure provides for, and includes, a method for preserving reagent red blood cells (RBC) comprising obtaining red blood cells, flushing the red blood cells with a gas comprising carbon monoxide to prepare carbon monoxide saturated hemoglobin in RBCs (CO-Hb RBCs) and storing the CO-Hb RBCs under anaerobic conditions in the presence of carbon monoxide (CO), wherein surface antigens of said CO-Hb RBCs are stabilized.

The present disclosure provides for, and includes, a method for preserving reagent red blood cells (RBC) comprising: obtaining red blood cells; treating the red blood cell with a chemical agent to prepare a red blood cell comprising a hemoglobin derivative; and storing the red blood cells comprising a hemoglobin derivative under anaerobic conditions to form reagent red blood cells, wherein surface antigens of the reagent red blood cells comprising a hemoglobin derivative are stabilized.

The present disclosure provides for, and includes, kits comprising one or more vials of carbon monoxide saturated RBCs (CO-Hb RBCs) having a plurality of CO-Hb RBCs having a common set of surface antigens in a buffer and instructions for use.

The present disclosure provides for, and includes, a vial of carbon monoxide saturated RBCs (CO-Hb RBCs) comprising a buffer and CO-Hb RBCs selected from the group consisting of: CO-Hb RBCs that are blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s; CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a}; CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a} and negative for the surface antigens Js^{a}, Kp^{a}, Wr^{a}, Di^{a}, V^{w}, V, Lu^{a}, and C^{w}; CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b}; CO-Hb RBCs are type-O cells that are positive for the Rh antigens D, C, and e; CO-Hb RBCs are type-O cells that are positive for the Rh antigens D, C, and e, I, Lu^{b}, Js^{b}, Kp^{b}, and Yt^{a}; CO-Hb RBCs are type-O cells that are positive for the Rh antigens D, C, and e, I, Lu^{b}, Js^{b}, Kp^{b}, and Yt^{a}, and that are negative for the surface antigens Js^{a}, Kp^{a}, Wr^{a}, Di^{a} V^{w}, V, Lu^{a} and C^{w}; CO-Hb RBCs are type-A cells that are positive for the surface antigen A1; CO-Hb RBCs are type-A cells that are positive for the surface antigen A1 and are negative for surface antigens D, C, and E; CO-Hb RBCs are type-A cells that are positive for the surface antigen A2; CO-Hb RBCs are type-A cells that are positive for the surface antigen A2; and are negative for surface antigens D, C, and E; CO-Hb RBCs are type-B cells that are positive for the surface antigen B; CO-Hb RBCs are type-B cells that are positive for the surface antigen B and are negative for surface antigens D, C, and E; CO-Hb RBCs are type-O cells that are positive for the surface antigens D, C, and e and having the Rh phenotype R₁R₁; CO-Hb RBCs are type-O cells that are positive for the surface antigens D, C^{w}, and e and having the Rh phenotype R₁^{w}R₁; CO-Hb RBCs are type-O cells that are positive for the surface antigens D, c, and E and having the Rh phenotype R₂R₂; CO-Hb RBCs are type-O cells that are positive for the surface antigens d, c, and e and having the Rh phenotype rr; CO-Hb RBCs are type-O cells that are positive for the surface antigens D, C, and e and having the Rh phenotype R₁R₁ and are positive for the surface antigens Lu^{b}, Js^{b}, Kp^{b}, and Yt^{a}; CO-Hb RBCs are type-O cells that are positive for the surface antigens D, C^{w}, and e and having the Rh phenotype R₁^{w}R₁ and are positive for the surface antigens Lu^{b}, Js^{b}, Kp^{b}, and Yt^{a}; CO-Hb RBCs are type-O cells that are positive for the surface antigens D, c, and E and having the Rh phenotype R₂R₂ and are positive for the surface antigens Lu^{b}, Js^{b}, Kp^{b}, and Yt^{a}; CO-Hb RBCs are type-O cells that are positive for the surface antigens d, c, and e and having the Rh phenotype rr and are positive for the surface antigens Lu^{b}, Js^{b}, Kp^{b}, and Yt^{a}; CO-Hb RBCs are type-O cells that are positive for the surface antigens D, C, and e and having the Rh phenotype R₁R₁ and are negative for the surface antigens Js^{a}, Kp^{a}, Wr^{a}, Di^{a}, V^{w}, V, Lu^{a} and C^{w}; CO-Hb RBCs are type-O cells that are positive for the surface antigens D, C^{w}, and e and having the Rh phenotype R₁^{w}R₁ and are negative for the surface antigens Js^{a}, Kp^{a}, Wr^{a}, Di^{a}, V^{w}, V, Lu^{a} and C^{w}; CO-Hb RBCs are type-O cells that are positive for the surface antigens D, c, and E and having the Rh phenotype R₂R₂ and are negative for the surface antigens Js^{a}, Kp^{a}, Wr^{a}, Di^{a}, V^{w}, V, Lu^{a} and C^{w}; CO-Hb RBCs are type-O cells that are positive for the surface antigens d, c, and e and having the Rh phenotype rr and are negative for the surface antigens Js^{a}, Kp^{a}, Wr^{a}, Di^{a}, V^{w}, V, Lu^{a} and C^{w}; CO-Hb RBCs are type-O cells that are positive for the surface antigens D, C, and e and having the Rh haplotype R₁ or are positive for the surface antigens D, c, and e and having the Rh haplotype R₂; CO-Hb RBCs are type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R₂ CO-Hb RBCs and are positive for the surface antigens Lu^{b}, Js^{b}, Kp^{b}, and Yt^{a}; CO-Hb RBCs are type-O cells that are positive for the surface antigens D, C, and e and having the Rh haplotype R₁ and are negative for the surface antigens Js^{a}, Kp^{a}, Wr^{a}, Di^{a}, V^{w}, V, Lu^{a} and C^{w} or are positive for the surface antigens D, c, and e and having the Rh haplotype R² and are negative for the surface antigens Js^{a}, Kp^{a}, Wr^{a}, Di^{a}, V^{w}, V, Lu^{a} and C^{w}; CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a}; CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens; CO-Hb RBCs are type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a}; CO-Hb RBCs are type-O cells that are positive for binding of antibodies to the D antigen and said cells are sensitized with anti-D(Rh₀) serum; CO-Hb RBCs are type-A cells that are positive for binding of antibodies to the A₂ antigen; CO-Hb RBCs are type-B cells that are positive for binding of antibodies to the B antigen and are negative for binding of anti-D(Rh₀) antibodies; CO-Hb RBCs are type-A cells that are positive for binding of antibodies to the Aᵢ antigen and are negative for binding of anti-D(Rh₀) antibodies; CO-Hb RBCs are type-AB cells that are positive for binding of antibodies to the A₁, B antigens and the Rh antigens d, c, and e of Rh blood group rr (dce/dec); CO-Hb RBCs are type-O cells that are positive for binding of antibodies to the Rh antigens D, d, C, c, and e and having the Rh phenotype R₁r (DCe/dce); CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b}; CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens; and CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, V^{w}, V, Lu^{a} and C^{w} antigens.

The present disclosure provides for, and includes a method for increasing the shelf-life of a red blood cell composition for drug delivery comprising obtaining a red blood cell comprising a pharmaceutical agent; treating the red blood cell with a chemical agent to prepare a red blood cell comprising a hemoglobin derivative; and storing the red blood cell comprising the pharmaceutical agent under a storage atmosphere.

The present disclosure also provides for, and includes, a method for increasing the shelf-life of a red blood cell composition for drug delivery comprising purging red blood cells comprising a pharmaceutical agent with carbon monoxide; and storing the purged red blood cells for a period of time.

The present disclosure provides for, and includes, a pharmaceutical composition comprising a leukocyte-depleted red blood cell expressing a pharmaceutical agents (RBC+PA), wherein the pharmaceutical composition comprises a non-oxygen hemoglobin binding agent and less than 25% S02.

The present disclosure provides for, and includes, a storage vial comprising a carbon monoxide saturated pharmaceutical composition comprising a red blood cell comprising a pharmaceutical agent.

The present disclosure also provides for, and includes, a method of packaging a predetermined dose of a red blood cell medication comprising: depleting oxygen by gas exchange with carbon monoxide; filing a medicament container with a predetermined dose of the red blood cell medication; and sealing the medicament container.

The present disclosure further provides for, and includes, a method for increasing the circulation life of a red blood cell for drug delivery comprising: obtaining a red blood cell comprising a pharmaceutical agent (RBC+PA); treating the red blood cell with a chemical agent to prepare a red blood cell comprising a hemoglobin derivative; and storing the red blood cell comprising the pharmaceutical agent under a storage atmosphere.

### BRIEF DESCRIPTION OF THE DRAWING

FIG. 1 is a graph showing the deoxygenation and carbon monoxidization of a red cell concentrate in an embodiment according to Example 2(a).

### DETAILED DESCRIPTION OF THE INVENTION

Blood group serology requires the determination of blood cell compatibility between a blood donor and a patient recipient before a transfusion or organ transplant involving the patient. Blood cell compatibility is determined by the non-occurrence of an immunological reaction between antibodies contained in the blood serum of a patient and antigens present on blood cells from the donor. Tests for blood cell typing and compatibility are generally of two types: (1) agglutination tests which determine whether a specific antibody added to the cells will cause their agglutination, and (2) cell lysis tests which determine whether a specific antibody added to the tested cells together with serum complement results in hemolysis. In blood cell typing and compatibility test procedures commonly used, both agglutination and cell lysis tests are carried out either manually by a trained technician or using automated devices. The presence of an immunological reaction is incompatible with transfusion and transplantation therapies.

The International Society of Blood Transfusion lists 33 blood group systems representing over 300 antigens. See Lögdberg et al. “Human blood group genes 2004: Chromosomal locations and cloning strategies,” Transfus Med Rev. 19:45-57 (2005), and Lögdberg et al., “Human blood group genes 2010: Chromosomal locations and cloning strategies revisited,” Transfus Med Rev. 25:36-46 (2011). Cloning and sequencing demonstrates that the genes of these blood group systems are autosomal, except XG and XK which are X-borne, and MIC2 which is present on both X and Y chromosomes. Many different blood group antigens are found on the surface of the red blood cells of every individual. These antigens, the products of inherited genes, exist in combinations that are likely to be unique between all individuals except identical twins.

A number of significant blood group systems are well known in the art and are presented in Table 1.

TABLE 1
Common Blood Groups

| Name | Symbol | Antigen # | Gene Name | Chromosome |
|---|---|---|---|---|
| ABO | ABO | 5 | ABO | 9 |
| MNS | MNS | 43 | GYPA, GYPB, | 4 |
| | | | GYPE | |
| P | P1 | 1 | P1 | 22 |
| Rhesus | Rh | 49 | RhD, RhCE | 1 |
| Lutheran | LU | 20 | LU | 19 |
| Kell | KEL | 25 | KEL | 7 |
| Lewis | LE | 6 | FUT3 | 19 |
| Duffy | FY | 6 | FY | 1 |
| Kidd | Jk | 3 | SLC14A1 | 18 |

www(dot)ncbi(dot)nlm(dot)nih(dot)gov/pmc/articles/PMC4260296/

Blood grouping is generally the process of testing red cells to determine which antigens are present and which are absent, normally utilizing antibodies to the antigen tested for. Additionally, when a person does not have a particular red cell antigen on his or her red blood cells, his or her serum may contain an antibody to that antigen. Whether or not the antibody is present in the serum depends on whether the person's immune system has been previously challenged by, and responded to, that specific antigen or something very similar to it. For example, a person whose red blood cells are Type A, i.e., having “A” antigens on the red cells, will have anti-B antibodies in his or her serum. Thus, if such a person is given type B blood, an immunological reaction will occur with possible serious clinical consequences.

Before transfusion therapy, the collected blood is tested for compatibility by analyzing the blood groups. Testing of blood groups is carried out by testing RBCs for the various antigens (A, B, etc.) and testing the serum for antibodies to the antigens. For the former test, RBCs from the sample blood is incubated with antibodies the recognize each of the blood group antigens and scored for binding either using aggregation or other known immunological approach. Testing of the serum can be performed in a variety of ways such as by ELISA where the antigen is provided, and antibody binding is detected indirectly through an enzyme or fluorescent reporter. A more traditional approach is to employ RBCs previously characterized as expressing specific antigens in agglutination assays called hemagglutination assays. In short, the agglutination assay comprises mixing reagent RBCs together with serum or plasma from the test sample of blood. Antibodies in the test sample, typically IgM having five antigen binding sites cross-link cells together causing clumping that can be seen macroscopically. Divalent IgG antibodies are also suitable for agglutination assays. Agglutination assays, including those directed to blood typing are well known in the art. See Löw et al., “Antiglobulin test in low-ionic strength salt solution for rapid antibody screening and cross-matching,” Vox Sang 26:53-61 (1974); Malyska et al., “The gel test,” Laboratory Medicine 25:81 (1994); and Technical manual. 14th ed. Bethesda, Md.: American Association of Blood Banks, 2002.

Among the more common Reagent RBCs typically used for reverse typing have on their surface either A, A, B or no ABO antigens (Type A1, Type A2, Type B, Type O). These cells are useful for detecting preformed antibodies which will cause agglutination of the reagent RBCs. For forward type testing, monoclonal anti-A and anti-B are used to detect the presence of their respective antigen on a sample red cell surface. Another well-known blood group is the Rh blood group having 58 different antigenic types, though nine types are the most common. The blood groups and the designations of the antigens are presented in Table 2 and Table 3. Byrne et al., “Review: other blood group systems—Diego, Yt, Xg, Scianna, Dombrock, Colton, Landsteiner-Wiener, and Indian,” Immunohematology. 20(1):50-8 (2004); Daniels G., “The molecular genetics of blood group polymorphism,” Transpl Immunol. 14(3-4):143-53 (2005); Daniels G., “Functions of red cell surface proteins,” Vox Sang. 93(4):331-40 (2007); Eyler and Telen, “The Lutheran glycoprotein: a multifunctional adhesion receptor.” Transfusion 46(4):668-77 (2006); Palacajornsuk P., “Review: molecular basis of MNS blood group variants. Immunohematology,” 22(4):171-82 (2006); Quill E., “Medicine. Blood-matching goes genetic,” Science 14; 319(5869):1478-9 (2008); Westhoff C M., “The structure and function of the Rh antigen complex,” Semin Hematol 44(1):42-50 (2007); Westhoff and Reid, “Review: the Kell, Duffy, and Kidd blood group systems,” Immunohematology 20(1):37-49 (2004); and Yamamoto F., “Review: ABO blood group system—ABH oligosaccharide antigens, anti-A and anti-B, A and B glycosyltransferases, and ABO genes,” Immunohematology 20(l):3-22 (2004), each of which are hereby incorporated by reference in their entireties.

TABLE 2
Blood Groups MNS, RH, LU, KEL and DI

| | 1 | 2 | 4 | 5 | 6 | 10 |
|---|---|---|---|---|---|---|
| 1 | ABO | MNS | RH | LU | KEL | DI |
| 2 | A | M | D | Lu^{a} | K | Di^{a} |
| 3 | B | N | C | Lu^{b} | k | Di^{b} |
| 4 | A, B | S | E | Lu3 | Kp^{a} | Wr^{a} |
| 5 | A1 | s | c | Lu4 | Kp^{b} | Wr^{b} |
| 6 | | U | e | Lu5 | Ku | Wd^{a} |
| 7 | | He | f | Lu6 | Js^{a} | Rb^{a} |
| 8 | | Mi^{a} | Ce | Lu7 | Js^{b} | WARR |
| 9 | | M^{c} | C^{w} | Lu8 | | ELO |
| 10 | | Vw | C^{x} | Lu9 | | Wu |
| 11 | | Mur | V | | Ul^{a} | Bp^{a} |
| 12 | | M^{g} | E^{w} | Lu11 | K11 | Mo^{a} |
| 13 | | Vr | G | Lu12 | K12 | Hg^{a} |
| 14 | | M^{c} | | Lu13 | K13 | Vg^{a} |
| 15 | | Mt^{a} | | Lu14 | K14 | Sw^{a} |
| 16 | | St^{a} | | | | BOW |
| 17 | | Ri^{a} | | Lu16 | K16 | NFLD |
| 18 | | Cl^{a} | Hrₒ | Lu17 | K17 | Jn^{a} |
| 19 | | Ny^{a} | Hr | Au^{a} | K18 | KREP |
| 20 | | Hut | hr^{S} | Au^{b} | K19 | Tr^{a} |
| 21 | | Hil | VS | Lu20 | Km | Fr^{a} |
| 22 | | M^{v} | C^{G} | Lu21 | Kp^{c} | SW1 |
| 23 | | Far | CE | | K22 | |
| 24 | | s^{D} | D^{w} | | K23 | |
| 25 | | Mit | | | K24 | |
| 26 | | Dantu | | | VLAN | |
| 27 | | Hop | c-like | | TOU | |
| 28 | | Nob | cE | | RAZ | |
| 29 | | En^{a} | hr^{H} | | VONG | |
| 30 | | En^{a}KT | Rh29 | | KALT | |
| 31 | | ‘N’ | Go^{a} | | KTIM | |
| 32 | | Or | hr^{B} | | KYO | |
| 33 | | DANE | Rh32 | | KUCI | |
| 34 | | TSEN | Rh33 | | KANT | |
| 35 | | MINY | Hr^{B} | | KASH | |
| 36 | | MUT | Rh35 | | | |
| 37 | | SAT | Be^{a} | | | |
| 38 | | ERIK | Evans | | | |
| 39 | | Os^{a} | | | | |
| 40 | | ENEP | Rh39 | | | |
| 41 | | ENEH | Tar | | | |
| 42 | | HAG | Rh41 | | | |
| 43 | | ENAV | Rh42 | | | |
| 44 | | MARS | Crawford | | | |
| 45 | | ENDA | Nou | | | |
| 46 | | ENEV | Riv | | | |
| 47 | | MNTD | Sec | | | |
| 48 | | | Dav | | | |
| 49 | | | JAL | | | |
| 50 | | | STEM | | | |
| 51 | | | FPTT | | | |
| 52 | | | MAR | | | |
| 53 | | | BARC | | | |
| 54 | | | JAHK | | | |
| 55 | | | DAK | | | |
| 56 | | | LOCR | | | |
| 57 | | | CENR | | | |
| 58 | | | CEST | | | |

TABLE 3
Blood Group Antigens

| | | Antigen Number | | | | | | | | | | | | | | |
|---|---|---|---|---|---|---|---|---|---|---|---|---|---|---|---|---|
| System | | 1 | 2 | 3 | 4 | 5 | 6 | 7 | 8 | 9 | 10 | 11 | 12 | 13 | 14 | 15 |
| 3 | P | P1 | | | | | | | | | | | | | | |
| 7 | LE | Le^{a} | Le^{b} | Le^{ab} | Le^{bH} | ALe^{b} | BLe^{b} | | | | | | | | | |
| 8 | FY | Fy^{a} | Fy^{b} | Fy3 | Fy4 | Fy5 | Fy6 | | | | | | | | | |
| 9 | JK | Jk^{a} | Jk^{b} | Jk3 | | | | | | | | | | | | |
| 11 | YT | Yt^{a} | Yt^{b} | | | | | | | | | | | | | |
| 12 | XG | Xg^{a} | CD99 | | | | | | | | | | | | | |
| 13 | SC | Sc1 | Sc2 | Sc3 | Rd | STAR | SCER | SCAN | | | | | | | | |
| 14 | DO | Do^{a} | Do^{b} | Gy^{a} | Hy | Jo^{a} | DOYA | | | | | | | | | |
| 15 | CO | Co^{a} | Co^{b} | Co3 | | | | | | | | | | | | |
| 16 | LW | | | | | LW^{a} | LW^{ab} | LW^{b} | | | | | | | | |
| 17 | CH/RG | Ch1 | Ch2 | Ch3 | Ch4 | Ch5 | Ch6 | WH | | | | Rg1 | Rg2 | | | |
| 18 | H | H | | | | | | | | | | | | | | |
| 19 | XK | Kx | | | | | | | | | | | | | | |
| 20 | GE | | Ge2 | Ge3 | Ge4 | Wb | Ls^{a} | An^{a} | Dh^{a} | GEIS | | | | | | |
| 21 | CROM | Cr^{a} | Tc^{a} | Tc^{b} | Tc^{c} | Dr^{a} | Es^{a} | IFC | WES^{a} | WES^{b} | UMC | GUTI | SERF | ZENA | CROV | CRAM |
| 22 | KN | Kn^{a} | Kn^{b} | McC^{a} | Sl1 | Yk^{a} | McC^{b} | Sl2 | Sl3 | KCAM | | | | | | |
| 23 | IN | In^{a} | In^{b} | INFI | INJA | | | | | | | | | | | |
| 24 | OK | Ok^{a} | | | | | | | | | | | | | | |
| 25 | RAPH | MER2 | | | | | | | | | | | | | | |
| 26 | JMH | JMH | JMHK | JMHL | JMHG | JMHM | | | | | | | | | | |
| 27 | I | I | | | | | | | | | | | | | | |
| 28 | GLOB | P | | | | | | | | | | | | | | |
| 29 | GIL | GIL | | | | | | | | | | | | | | |
| 30 | RHAG | Duclos | Ol^{a} | Duclos- | | | | | | | | | | | | |
| | | | | like | | | | | | | | | | | | |

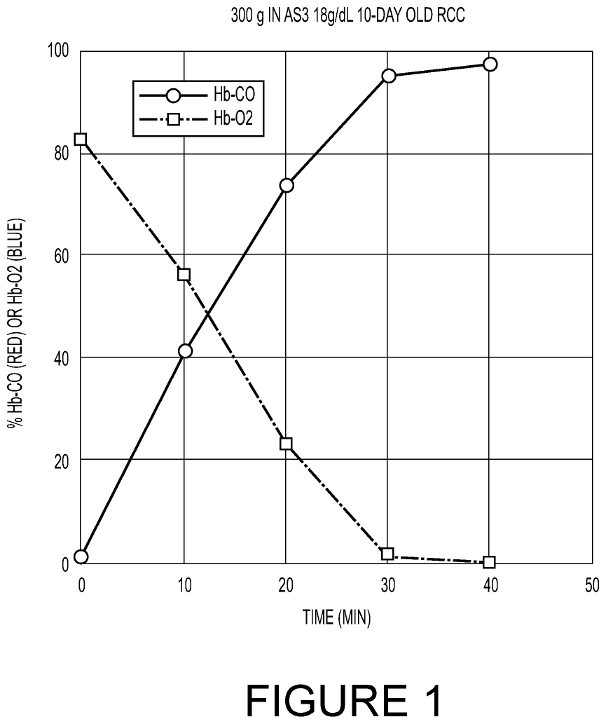

The present disclosure provides for, and includes, methods to reduce degradation of blood group antigens during storage. More specifically, the present disclosure provides for reducing the formation of storage lesions in RBCs during storage including, but not limited to, ROS induced lesions. In brief, RBCs are collected exposed to an atmosphere of carbon monoxide (CO) for a period of time sufficient to remove oxygen (O₂) bound to the heme group of hemoglobin. As shown in Example 2 and FIG. 1, the exchange of oxygen for CO occurs rapidly and essentially complete in 30 minutes and three exchanges of gas. Methods that bring the CO into contact with the blood, particularly those that increase the surface to volume ration of the CO/liquid interface would significantly reduce the length of time necessary to exchange the bound oxygen with carbon monoxide. As discussed above, carbon monoxide has a significantly higher affinity for hemoglobin that oxygen (e.g., 400×), thus exposure of RBCs to CO at any level and time will begin the exchange process and provided sufficient CO, will drive the exchange for completion.

The present disclosure provides for, includes, but is not limited to, exchanging the oxygen for carbon monoxide according the methods shown in Example 2. In an aspect, red cell concentrate (RCC, also known as packed red blood cells) are held in a container and CO is introduced and the container is shaken or mixed gently for a period. In an aspect, the container is a standard blood storage bag comprising polyvinyl chloride. In an aspect, the CO gas is replaced and the mixing repeated one or more time until the hemoglobin is saturated with CO (e.g., Hb-CO RBCs are produced). In another aspect, the container containing the RCCs are provided with a volume of CO and left overnight with, or without, occasional mixing. Mixing improves the rate of exchange, but is not required. In another aspect, the blood and CO is separated by a gas permeable membrane. This can be done using methods known in the art, for example using a Sorin D100 oxygenator and providing CO rather than oxygen. The advantage of a membrane based approach is that the gas can be continuously replaced thereby maintaining the concentration gradient and increasing the rate of exchange.

In another aspect, CO gas can be bubbled through a container or bag of RBCs. Not to be limited by theory, it is thought that by maintaining the bubbles to less than 1 μm in diameter, lysis of the red cells can be prevented or minimized. The ‘bubbling’ method shares the same advantages as the membrane based approach as the CO bubble will provide for a maximal concentration difference thereby facilitating the kinetics of the exchange reaction. The bubble approach also provides for the further advantage of mixing the RBCs.

While it may be preferable to perform carbon monoxide exchange on RCCs, the specification provides for, and includes, exchanging CO for oxygen on blood at any stage of the process. In an aspect, the CO is exchanged on whole blood, prior to removing for example platelets or leukocytes. In an aspect, CO is exchanged on leukoreduced blood. The methods of the present specification can be applied to RBCs prepared by apheresis or collected using traditional methods. The methods may also be performed after processing of the RBCs into a suitable buffer for reagent use and storage. See for example, U.S. Patent Publication No. 2011/0045455, published Feb. 26, 2001; and International Patent Publication No. WO 1983/003477, published Oct. 13, 1983. Other storage solutions compatible with blood typing methods are known in the art.

The present specification provides for, and includes, methods for preparing CO-Hb RBCs that further includes storing said CO-Hb RBCs under anaerobic conditions in the presence of CO. In an aspect, the cells are prepared as described above and transferred to a vial under an atmosphere comprising carbon monoxide. In another aspect, the CO-Hb RBCs are transferred to a container for storage having a nitrogen atmosphere. As provided herein, during storage, any suitable non-oxygen containing gas is suitable. In certain aspect, additional CO is provided before sealing the container for CO-Hb RBCs storage.

The present specification provides for, and includes, methods for preparing CN-Hb RBCs that further includes storing said CN-Hb RBCs under anaerobic conditions. In an aspect, the cells are prepared as described above and transferred to a vial under an atmosphere comprising ambient air. In another aspect, the CN-Hb RBCs are transferred to a container for storage having a nitrogen atmosphere. As provided herein, during storage, any suitable non-oxygen containing gas is suitable. In certain aspect, additional non-oxygen containing gas is provided before sealing the container for CN-Hb RBCs storage.

The present specification provides for, and includes, methods for preparing N₃-Hb RBCs that further includes storing said N₃-Hb RBCs under anaerobic conditions. In an aspect, the cells are prepared as described above and transferred to a vial under an atmosphere comprising ambient air. In another aspect, the N₃-Hb RBCs are transferred to a container for storage having a nitrogen atmosphere. As provided herein, during storage, any suitable non-oxygen containing gas is suitable. In certain aspect, additional non-oxygen containing gas is provided before sealing the container for N₃-Hb RBCs storage.

The present disclosure provides for, and includes, a method for preserving reagent red blood cells (RBC) comprising: obtaining red blood cells; treating the red blood cell with a chemical agent to prepare a red blood cell comprising a hemoglobin derivative; and storing the reagent red blood cells comprising a hemoglobin derivative under anaerobic conditions to form reagent red blood cells, wherein surface antigens of the reagent red blood cells comprising a hemoglobin derivative are stabilized.

In an aspect, the chemical agent is carbon monoxide (CO) and said hemoglobin derivative is carboxy-hemoglobin (CO-Hb). In another aspect, the chemical agent is cyanide and said hemoglobin derivative is cyano-methemoglobin (CN-Hb). In another aspect, the chemical agent is azide (N3) and said hemoglobin derivative is azido-methemoglobin (N₃—Hb) prepared by reacting with an aqueous solution of sodium azide.

The present disclosure provides for, and includes, kits comprising one or more vials of cyanide or azide saturated RBCs (CN-Hb RBCs or N3-Hb RBCs) having a plurality of CN-Hb RBCs or N3-Hb RBCs having a common set of surface antigens in a buffer and instructions for use.

The present disclosure provides for, and includes, a vial of cyanide or azide saturated RBCs (CN-Hb RBCs or N3-Hb RBCs) comprising a buffer and CN-Hb RBCs or N3-Hb RBCs selected from the group consisting of: CN-Hb RBCs or N3-Hb RBCs that are blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s; CN-Hb RBCs or N3-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a}. CN-Hb RBCs or N3-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a} and negative for the surface antigens Js^{a}, Kp^{a}, Wr^{a}, Di^{a}, V^{w}, V, Lu^{a}, and C^{w}; CN-Hb RBCs or N3-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b}; CN-Hb RBCs or N3-Hb RBCs are type-O cells that are positive for the Rh antigens D, C, and e; CN-Hb RBCs or N3-Hb RBCs are type-O cells that are positive for the Rh antigens D, C, and e, I, Lu^{b}, Js^{b}, Kp^{b}, and Yt^{a}; CN-Hb RBCs or N3-Hb RBCs are type-O cells that are positive for the Rh antigens D, C, and e, I, Lu^{b}, Js^{b}, Kp^{b}, and Yt^{a}, and that are negative for the surface antigens Js^{a}, Kp^{a}, Wr^{a}, Di^{a} V^{w}, V, Lu^{a} and C^{w}; CN-Hb RBCs or N3-Hb RBCs are type-A cells that are positive for the surface antigen A1; CN-Hb RBCs or N3-Hb RBCs are type-A cells that are positive for the surface antigen A1 and are negative for surface antigens D, C, and E; CN-Hb RBCs or N3-Hb RBCs are type-A cells that are positive for the surface antigen A2; CN-Hb RBCs or N3-Hb RBCs are type-A cells that are positive for the surface antigen A2; and are negative for surface antigens D, C, and E; CN-Hb RBCs or N3-Hb RBCs are type-B cells that are positive for the surface antigen B; CN-Hb RBCs or N3-Hb RBCs are type-B cells that are positive for the surface antigen B and are negative for surface antigens D, C, and E; CN-Hb RBCs or N3-Hb RBCs are type-O cells that are positive for the surface antigens D, C, and e and having the Rh phenotype R₁R₁; CN-Hb RBCs or N3-Hb RBCs are type-O cells that are positive for the surface antigens D, C^{w}, and e and having the Rh phenotype R₁^{w}R₁; CN-Hb RBCs or N3-Hb RBCs are type-O cells that are positive for the surface antigens D, c, and E and having the Rh phenotype R₂R₂; CN-Hb RBCs or N3-Hb RBCs are type-O cells that are positive for the surface antigens d, c, and e and having the Rh phenotype rr; CN-Hb RBCs or N3-Hb RBCs are type-O cells that are positive for the surface antigens D, C, and e and having the Rh phenotype R₁R₁ and are positive for the surface antigens Lu^{b}, Js^{b}, Kp^{b}, and Yt^{a}; CN-Hb RBCs or N3-Hb RBCs are type-O cells that are positive for the surface antigens D, C^{w}, and e and having the Rh phenotype R₁^{w}R₁ and are positive for the surface antigens Lu^{b}, Js^{b}, Kp^{b}, and Yt^{a}; CN-Hb RBCs or N3-Hb RBCs are type-O cells that are positive for the surface antigens D, c, and E and having the Rh phenotype R₂R₂ and are positive for the surface antigens Lu^{b}, Js^{b}, Kp^{b}, and Yt^{a}; CN-Hb RBCs or N3-Hb RBCs are type-O cells that are positive for the surface antigens d, c, and e and having the Rh phenotype rr and are positive for the surface antigens Lu^{b}, Js^{b}, Kp^{b}, and Yt^{a}; CN-Hb RBCs or N3-Hb RBCs are type-O cells that are positive for the surface antigens D, C, and e and having the Rh phenotype R₁R₁ and are negative for the surface antigens Js^{a}, Kp^{a}, Wr^{a}, Di^{a}, V^{w}, V, Lu^{a} and C^{w}; CN-Hb RBCs or N3-Hb RBCs are type-O cells that are positive for the surface antigens D, C^{w}, and e and having the Rh phenotype R₁^{w}R₁ and are negative for the surface antigens Js^{a}, Kp^{a}, Wr^{a}, Di^{a}, V^{w}, V, Lu^{a} and C^{w}; CN-Hb RBCs or N3-Hb RBCs are type-O cells that are positive for the surface antigens D, c, and E and having the Rh phenotype R₂R₂ and are negative for the surface antigens Js^{a}, Kp^{a}, Wr^{a}, Di^{a}, V^{w}, V, Lu^{a} and C^{w}; CN-Hb RBCs or N3-Hb RBCs are type-O cells that are positive for the surface antigens d, c, and e and having the Rh phenotype rr and are negative for the surface antigens Js^{a}, Kp^{a}, Wr^{a}, Di^{a}, V^{w}, V, Lu^{a} and C^{w}; CN-Hb RBCs or N3-Hb RBCs are type-O cells that are positive for the surface antigens D, C, and e and having the Rh haplotype R₁ or are positive for the surface antigens D, c, and e and having the Rh haplotype R²; CN-Hb RBCs or N3-Hb RBCs are type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R₂ CN-Hb RBCs or N3-Hb RBCs and are positive for the surface antigens Lu^{b}, Js^{b}, Kp^{b}, and Yt^{a}; CN-Hb RBCs or N3-Hb RBCs are type-O cells that are positive for the surface antigens D, C, and e and having the Rh haplotype R₁ and are negative for the surface antigens Js^{a}, Kp^{a}, Wr^{a}, Di^{a}, V^{w}, V, Lu^{a} and C^{w} or are positive for the surface antigens D, c, and e and having the Rh haplotype R² and are negative for the surface antigens Js^{a}, Kp^{a}, Wr^{a}, Di^{a}, V^{w}, V, Lu^{a} and C^{w}; CN-Hb RBCs or N3-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a}; CN-Hb RBCs or N3-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens; CN-Hb RBCs or N3-Hb RBCs are type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a}; CN-Hb RBCs or N3-Hb RBCs are type-O cells that are positive for binding of antibodies to the D antigen and said cells are sensitized with anti-D(Rh₀) serum; CN-Hb RBCs or N3-Hb RBCs are type-A cells that are positive for binding of antibodies to the A₂ antigen; CN-Hb RBCs or N3-Hb RBCs are type-B cells that are positive for binding of antibodies to the B antigen and are negative for binding of anti-D(Rh₀) antibodies; CN-Hb RBCs or N3-Hb RBCs are type-A cells that are positive for binding of antibodies to the A₁ antigen and are negative for binding of anti-D(Rh₀) antibodies; CN-Hb RBCs or N3-Hb RBCs are type-AB cells that are positive for binding of antibodies to the A₁, B antigens and the Rh antigens d, c, and e of Rh blood group rr (dce/dec); CN-Hb RBCs or N3-Hb RBCs are type-O cells that are positive for binding of antibodies to the Rh antigens D, d, C, c, and e and having the Rh phenotype R₁r (DCe/dce); CN-Hb RBCs or N3-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b}; CN-Hb RBCs or N3-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens; and CN-Hb RBCs or N3-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, V^{w}, V, Lu^{a} and C^{w} antigens.

The present disclosure provides for, and includes, a method of preserving reagent red blood cells (RBC) comprising obtaining red blood cells that are selected from, but not limited to the following 40 immunological types:
1. Blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s;
2. Blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a};
3. Blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a} and negative for the surface antigens Js^{a}, Kp^{a}, Wr^{a}, Di^{a}, V^{w}, V, Lu^{a}, and C^{w};
4. Blood type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b};
5. Blood type-O cells that are positive for the Rh antigens D, C, and e;
6. Blood type-O cells that are positive for the Rh antigens D, C, and e, I, Lu^{b}, Js^{b}, Kp^{b}, and Yt^{a};
7. Blood type-O cells that are positive for the Rh antigens D, C, and e, I, Lu^{b}, Js^{b}, Kp^{b}, and Yt^{a}, and that are negative for the surface antigens Js^{a}, Kp^{a}, Wr^{a}, Di^{a} V^{w}, V, Lu^{a} and C^{w};
8. Blood type-A cells that are positive for the surface antigen A1;
9. Blood type-A cells that are positive for the surface antigen A1 and are negative for surface antigens D, C, and E;
10. Blood type-A cells that are positive for the surface antigen A2;
11. Blood type-A cells that are positive for the surface antigen A2; and are negative for surface antigens D, C, and E;
12. Blood type-B cells that are positive for the surface antigen B;
13. Blood type-B cells that are positive for the surface antigen B and are negative for surface antigens D, C, and E;
14. Blood type-O cells that are positive for the surface antigens D, C, and e and having the Rh phenotype R₁R₁;
15. Blood type-O cells that are positive for the surface antigens D, C^{w}, and e and having the Rh phenotype R₁^{w}R₁;
16. Blood type-O cells that are positive for the surface antigens D, c, and E and having the Rh phenotype R₂R₂;
17. Blood type-O cells that are positive for the surface antigens d, c, and e and having the Rh phenotype rr;
18. Blood type-O cells that are positive for the surface antigens D, C, and e and having the Rh phenotype R₁R₁ and are positive for the surface antigens Lu^{b}, Js^{b}, Kp^{b}, and Yt^{a};
19. Blood type-O cells that are positive for the surface antigens D, C^{w}, and e and having the Rh phenotype R₁^{w}R₁ and are positive for the surface antigens Lu^{b}, Js^{b}, Kp^{b}, and Yt^{a};
20. Blood type-O cells that are positive for the surface antigens D, c, and E and having the Rh phenotype R₂R₂ and are positive for the surface antigens Lu^{b}, Js^{b}, Kp^{b}, and Yt^{a};
21. Blood type-O cells that are positive for the surface antigens d, c, and e and having the Rh phenotype rr and are positive for the surface antigens Lu^{b}, Js^{b}, Kp^{b}, and Yt^{a};
22. Blood type-O cells that are positive for the surface antigens D, C, and e and having the Rh phenotype R₁R₁ and are negative for the surface antigens Js^{a}, Kp^{a}, Wr^{a}, Di^{a}, V^{w}, V, Lu^{a} and C^{w};
23. Blood type-O cells that are positive for the surface antigens D, C^{w}, and e and having the Rh phenotype R₁^{w}R₁ and are negative for the surface antigens Js^{a}, Kp^{a}, Wr^{a}, Di^{a}, V^{w}, V, Lu^{a} and C^{w};
24. Blood type-O cells that are positive for the surface antigens D, c, and E and having the Rh phenotype R₂R₂ and are negative for the surface antigens Js^{a}, Kp^{a}, Wr^{a}, Di^{a}, V^{w}, V, Lu^{a} and C^{w};
25. Blood type-O cells that are positive for the surface antigens d, c, and e and having the Rh phenotype rr and are negative for the surface antigens Js^{a}, Kp^{a}, Wr^{a}, Di^{a}, Vw, V, Lu^{a} and C^{w};
26. Blood type-O cells that are positive for the surface antigens D, C, and e and having the Rh haplotype R₁ or are positive for the surface antigens D, c, and e and having the Rh haplotype R²;
27. Blood type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R₂ CO-Hb RBCs and are positive for the surface antigens Lu^{b}, Js^{b}, Kp^{b}, and Yt^{a};
28. Blood type-O cells that are positive for the surface antigens D, C, and e and having the Rh haplotype R₁ and are negative for the surface antigens Js^{a}, Kp^{a}, Wr^{a}, Di^{a}, V^{w}, V, Lu^{a} and C^{w} or are positive for the surface antigens D, c, and e and having the Rh haplotype R₂ and are negative for the surface antigens Js^{a}, Kp^{a}, Wr^{a}, Di^{a}, V^{w}, V, Lu^{a} and C^{w};
29. Blood type-O cells that have been ficin treated and that are negative of the surface antigens M, N, Fy^{a}, Fy^{b}, S, s, Xg^{a}, Pr, Ch^{a}, Rg^{a} and Yk^{a};
30. Blood type-O cells that have been ficin treated and that are negative of the surface antigens M, N, Fy^{a}, Fy^{b}, S, s, Xg^{a}, Pr, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens;
31. Blood type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a};
32. Blood type-O cells that are positive for binding of antibodies to the D antigen and said CO-Hb RBCs are sensitized with anti-D(Rh₀) serum;
33. Blood type-A cells that are positive for binding of antibodies to the A₂ antigen;
34. Blood type-B cells that are positive for binding of antibodies to the B antigen and are negative for binding of anti-D(Rh₀) antibodies;
35. CO-Hb RBCs are type-A cells that are positive for binding of antibodies to the A₁ antigen and are negative for binding of anti-D(Rh₀) antibodies;
36. Blood type-AB cells that are positive for binding of antibodies to the A₁, B antigens and the Rh antigens d, c, and e of Rh blood group rr (dce/dec);
37. Blood type-O cells that are positive for binding of antibodies to the Rh antigens D, d, C, c, and e and having the Rh phenotype R₁r (DCe/dce);
38. Blood type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b};
39. Blood type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens; and
40. Blood type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, V^{w}, V, Lu^{a} and C^{w} antigens.

In an aspect of the present disclosure, carbon monoxide treatment can be substituted with cyanide (CN) or azide (N₃). In an aspect, the present disclosure provides for, and includes, methods to reduce degradation of blood group antigens during storage. More specifically, the present disclosure provides for reducing the formation of storage lesions in RBCs during storage including, but not limited to, ROS induced lesions. In brief, RBCs are collected exposed to cyanide (CN) or azide (N₃) for a period of time sufficient to remove oxygen (O₂) bound to the heme group of hemoglobin. The present disclosure provides for methods to react cyanide with oxidized hemoglobin (methemoglobin) to form the very stable cyano-methemoglobin. The cyano-methemoglobin complex can be formed by numerous oxidizing agents such as methylene blue, phenylmehtylsulfate, and potassium ferricyanide. In another aspect, the present disclosure provides for methods to react azide with methemoglobin to form the stable azido-hemoglobin complex.

As would be understood to a person of ordinary skill in the art, the selection of RBCs suitable for the preparation of reagent RBCs is not limited to the combinations of blood types as provided above. A person or ordinary skill would recognize that RBCs having any antigen combination of the groups recited in Tables 2 and 3 are useful for the methods of the present application. Indeed, a person of ordinary skill in the art would recognize that any RBC, once characterized immunologically would be suitable for the preparation of Reagent RBCs preserved with carbon monoxide (Hb-CO RBCs), cyanide (Hb-CN RBCs), or azide (Hb-N₃ RBCs).

The present specification provides for, and includes, exchanging the oxygen bound on hemoglobin with carbon monoxide using any known methods in the art, including but not limited to the methods of shown in the examples. Thus, as used herein, the term ‘flushing’ refers to any method that brings carbon monoxide gas into contact with RBCs including bubbling or passing through a membrane.

The present specification provides for, and includes, kits comprising carbon monoxide stabilized red blood cells (Hb-CO RBCs). Kits of the present specification may include one or more of the 40 specific types Hb-CO RBCs of the cells described above, but are not limited to those cells. Other suitable Hb-CO RBCs can be prepared as needed. In certain aspects, the kits provide the cells suspended in a suitable buffer and the preparation of the cells may include various washing steps either before carbon monoxide exchange, or after carbon monoxide exchange. Additional reagents and buffer necessary for performing immunological assays may be included in the kits. In many aspects, the kits will include instructions on the performance of the assays, lot characterization of the Hb-CO RBCs and other materials pertinent to a person of skill in the art. In some aspect, the kits of the present specification may include various labware such as tubes, pipettes, buffers, etc.

The present specification provides for, and includes, containers containing the CO-Hb RBCs described herein. In an aspect, the present specification provides for, and includes, containers containing the CN-Hb RBCs described herein. In another aspect, the present specification provides for, and includes, containers containing the N₃-Hb RBCs described herein. In an aspect, the container is a vial. In another aspect, the container is a tube. In yet another aspect, the container is an ampule.

The present disclosure provides for, and includes a method for increasing the shelf-life of a red blood cell composition for drug delivery comprising obtaining a red blood cell comprising a pharmaceutical agent; treating the red blood cell with a chemical agent to prepare a red blood cell comprising a hemoglobin derivative; and storing the red blood cell comprising the pharmaceutical agent under a storage atmosphere.

The present disclosure provides for, and includes a method for increasing the shelf-life of a red blood cell composition for drug delivery comprising obtaining a red blood cell comprising a pharmaceutical agent and storing the red blood cell comprising a pharmaceutical agent in liquid nitrogen.

The present disclosure provides for a red blood cell comprising a pharmaceutical agent. In an aspect of the present disclosure, the pharmaceutical agent comprises a transgene expressed on the cell surface of the red blood cell. In another aspect, the pharmaceutical agent is localized in the cytosol of the red blood cell. In another aspect, the pharmaceutical agent is localized to the intracellular membrane. In yet another aspect, the pharmaceutical agent is a fusion protein. In a further aspect, the pharmaceutical agent comprises a protein selected from a protein as provided by tables B and C of US Patent Publication No. 2017/0020926, published Jan. 26, 2019, and the group consisting of those listed in Table 4, Table 5, Table 6, and Table 7. In another aspect, the pharmaceutical agent comprises an antigen. In another aspect, the antigen is expressed by fusion to a red blood cell protein. In yet another aspect, an antigen is selected from tables as provided by table F and G of US Patent Publication No. 2017/0020926, published Jan. 26, 2019, and the antigens listed in Table 8, Table 9, and Table 10. In another aspect, the pharmaceutical agent is a protein selected from the class of proteins listed in Table 11. In a further aspect, the pharmaceutical agent comprises an antigen or target for an antigen listed in Table 12. In yet another aspect, the pharmaceutical agent is to treat a disease, comprises an exogeneous antigen or target, or is rendered to target an exogeneous antigen or target provided by US Patent Publication No. 2018/0085402, published Mar. 29, 2018 and listed in Table 13. In another aspect, the pharmaceutical agent comprises an antibody molecule. In another aspect, the pharmaceutical agent inhibits an immune checkpoint molecule. In yet another aspect, the pharmaceutical agent comprises a first polypeptide on the red blood cell surface to promote fusion of the red blood cell with a target cell and a second polypeptide selected from any one of the polypeptides listed in Table 14, Table 15, and Table 16 In another aspect, the second polypeptide is formulated to activate or inhibit T cells as provided in Table 15 and Table 16.

TABLE 4
Circulating cell associated proteins

CD1
CD2
CD3
CD4
CD5
CD6
CD7
CD8
CD9
CD10
CD1la
CD1lb
CD11c
CD12w
CD13
CD14
CD15
CD16
CD17
CD18
CD19
CD20
CD21
CD22
CD23
CD24
CD25
CD26
CD27
CD28
CD29
CD30
CD31
CD32
CD33
CD34
CD35
CD36
CD37
CD38
CD39
CD40
CD41
CD42
CD43
CD44
CD45
CD46
CD47
CD48
CD49a
CD49b
CD49c
CD49d
CD49e
CD49f
CD53
CD54
CD55
CD56
CD57
CD58
CD59
CD61
CD62E
CD62L
CD62P
CD63
CD68
CD69
CD71
CD72
CD73
CD74
CD80
CD81
CD82
CD83
CD86
CD87
CD88
CD89
CD90
CD91
CD95
CD96
CD100
CD103
CD105
CD106
CD107
CD107a
CD107b
CD109
CD117
CD120
CD122
CD127
CD132
CD133
CD134
CD135
CD138
CD141
CD142
CD143
CD144
CD147
CD151
CD152
CD154
CD156
CD158
CD163
CD165
CD166
CD168
CD184
CD186
CD195
CD197
CD199
CD209
CD202a
CD220
CD221
CD235a
CD271
CD279
CD303
CD304
CD209
CD326
TLR1
TLR2
TLR4
TLR5
TLR6

TABLE 5
Erythrocyte associated proteins

| | | | |
|---|---|---|---|
| 2′,3′-cyclic- | Creatine kinase | Hypothetical protein | RAP1A or RAP1B |
| nucleotide 3′- | | XP_100510 | |
| phosphodiesterase | | | |
| Acetylcholinesterase | DC 38 | Hypothetical protein | RAP2B |
| | | XP_100619 | |
| Actin alpha and beta | Duodenal | Hypothetical protein | Rh blood D group |
| chain | cytochrome b | XP_100665 | antigen polypeptide |
| Adenosine deaminase | Enhancer protein | Hypothetical protein | Rhesus D category |
| | | XP_100925 | VI type III protein |
| Adducin alpha | Erythroblast | Hypothetical protein | Similar to adhesive |
| subunit | membrane- | XP_103707 | plaque matrix |
| | associated protein | | protein precursor |
| Aldolase A | Far upstream | Hypothetical protein | Similar to ankyrin 1 |
| | element binding | XP_106269 | |
| | protein | | |
| Ankyrin 1 isoform 2 | Flotillin 1 | Ig heavy chain V-V region | Similar to flotillin 2 |
| Ankyrin 1 isoform 4 | Flotillin 2 47 | Kell | Similar to |
| | | | glycophorin A |
| Ankyrin 1 splice | Glucose | KIAA0340 | Similar to Lutheran |
| form 2 | transporter | | blood group |
| | glycoprotein | | |
| Aquaporin 1 | Glutathione | KIAA1741 protein | Similar to RAS- |
| | transferase | | related protein |
| | | | RAB-15 |
| Arginase type 1 | Glyceraldehyde- | Lyn B protein | Similar to RAS- |
| | 3-phosphate | | related protein RAL- |
| | dehydrogenase | | A |
| Arginase type 1 | Glycophorin A | Membrane protein p55 | Similar to |
| erythroid variant | | | tropomyosin |
| ATP-binding cassette | Glycophorin A | Phosphatidylinositol-4- | Similar to |
| half-transporter | precursor | phosphate 5 kinase type | tropomyosin 4 18 |
| | | III | |
| ATP-binding cassette | Glycophorin C | Phosphoribosyl | Solute carrier family |
| subfamily C member 6 | isoform 1 | pyrophosphate synthetase | 29 (nucleoside |
| | | | transporter) member 1 |
| bA421I18.2 (novel | Hemoglobin | Poly (A)-specific | Solute carrier family 2 |
| protein) | alpha | ribonuclease | (facilitated glucose |
| | | | transporter) member 1 |
| B-CAM protein | Hemoglobin beta | Presenilin-associated | Spectrin alpha chain |
| | | protein | |
| Block of proliferation | Hemoglobin delta | Protein band 3 | Spectrin beta chain |
| 1 | | | |
| C-1 -tetrahydrofolate | Hemoglobin | Protein band 4.1 | Translation |
| synthase | epsilon | | initiation factor 2C |
| Calcium transposing | Hemoglobin | Protein band 4.1 | Tropomodulin |
| ATPase 4 | gamma | (elliptocytosis 1, RH- | |
| | | linked) | |
| CD55 | HGTD-P | Protein band 4.2 | Tropomyosin 3 |
| CD58 | Hypothetical | Protein band 4.9 (dematin) | Tropomyosin |
| | protein | | isoform |
| | XP_061743 or | | |
| | XP_089854 | | |
| CD59 antigen | Hypothetical | Protein band 7.2b, | Tropomyosinalpha |
| | protein | stomatin | chain (smooth |
| | XP_091430 | | muscle) 26 |
| Cell surface | Hypothetical | RAB 35 | Unknown protein |
| glycoprotein CD44 | protein | | |
| | XP_091724 | | |
| Channel-like integral | Hypothetical | Rabphilin-3 A-integrating | Vesicle-associated |
| membrane protein | protein | protein | membrane protein 2 |
| | XP_092517 | | (synaptobrevin 2) |
| Complement receptor | Hypothetical | Ral A binding protein | Zona pellucida |
| 1 | protein | | binding protein |
| | XP_095819 | | |
| Adipocyte plasma | Stomatin | Myosin-9 | Histone H1.1 |
| membrane-associated | | | |
| protein | | | |
| Ammonium | Stomatin-like | Protein 4.1 | Histone H2A type 1- |
| transporter Rh type A | protein | | B/E |
| Aquaporin 1 | Thioredoxin- | Spectrin alpha chain, | Histone 113.1 |
| | related | erythrocyte | |
| | transmembrane | | |
| | protein 4 | | |
| Aquaporin 7 | TMCC2 | Spectrin beta chain, | Histone 114 |
| | | erythrocyte | |
| ATP-binding cassette | Transferrin | Talin-1 | Lamin A/C |
| sub-family B member | receptor protein 1 | | |
| 6, mitochondrial | | | |
| Band 3 anion | Transmembrane | Talin-2 | Lamina-associated |
| transport protein | and coiled-coil | | polypeptide 2, |
| | domain family 2 | | isoform alpha |
| Basigin | Urea transporter 1 | Tropomodulin-1 | Lamina-associated |
| | | | polypeptide 2. |
| | | | isoforms |
| | | | beta/gamma |
| CD44 | Zinc transporter I | Tropomyosin I (Alpha) | Lamin-B receptor |
| | | isoform 4 | |
| CD47 | 55 kDa | Tropomyosin 3 | I.amin-B1 |
| | erythrocyte | | |
| | membrane protein | | |
| Equilibrative | Actin. alpha | Tropomyosin alpha-3 | Lamin-B2 |
| nucleoside transporter 1 | cardiac muscle | chain | |
| Erythroid membrane- | Actin, | Tubulin alpha-I chain | Matrin-3 |
| associated protein | cytoplasmic | | |
| Flotillin-1 | Actin-related | Tubulin beta chain | Multiple inositol |
| | protein 2 | | polyphosphate |
| | | | phosphatase 1 |
| Flotillin-2 | Actin-related | Tubulin, alpha 1 (‘Testis | N-acylneuraminate |
| | protein 2/3 | specific) | cytidylyltransferase |
| | complex subunit | | |
| | 1B | | |
| Glucose transporter, | Actin-related | Tubulin, alpha 8 | Neutral alpha- |
| type I | protein 2/3 | | glucosidase AB |
| | complex subunit | | |
| | 2 | | |
| Glycophorin-A | Actin-related | Tubulin, beta 6 | Nuclear pore |
| | protein 3 | | complex protein |
| | | | Nup93 |
| Glycophorin-B | Alpha-actinin-4 | Vinculin | Nuclear pore |
| | | | membrane |
| | | | glycoprotein 210 |
| Glycophorin-C | Alpha-adducin | 78 kDa glucose-regulated | Nucleolin |
| | | protein | |
| Immunoglobulin-like | Ankyrin-1 | Antigen KI-67 | Nucleoporin |
| domain-containing | | | NUP188 homolog |
| receptor 1 | | | |
| Integrin alpha-X | Ankyrin-3 | ATP-dependent RNA | Nucleoprotein TPR |
| | | helicase DDX39A | |
| Integrin beta-I | Beta-actin-like | Calnexin | Prelamin-A/C |
| | protein 2 | | |
| Kell blood group | Beta-adducin | Calreticulin | Protein disulfide- |
| glycoprotein | | | isomerase |
| Large neutral amino | Capping protein | DNA topoisomerase 1 | Protein disulfide- |
| acids transporter | (Actin filament) | | isomerase A4 |
| small subunit 3 | muscle Z-line, | | |
| | beta | | |
| Membrane transport | Cortactin | DNA-dependent protein | Protein disulfide- |
| protein XK | | kinase catalytic subunit | isomerase A6 |
| Membrane-associated | Dematin | Dolichyl- | Protein ERGIC-53 |
| progesterone receptor | | diphosphooligosaccharide- | |
| component 2 | | protein | |
| | | glycosyltransferase 48 | |
| | | kDa subunit | |
| Monocarboxylate | Dynactin 2 (P50), | Dolichyl- | Ribophorin II |
| transporter I | isoform CRA_b | diphosphooligosaccharide- | |
| | | protein | |
| | | glycosyltransferase | |
| | | subunit 1 | |
| Multidrug resistance- | Erythrocyte | Endoplasmic reticulum | Transitional |
| associated protein 4 | membrane protein | resident protein 29 | endoplasmic |
| | band 4.2 | | reticulum ATPase |
| Neutral cholesterol | Filamin-A | Endoplasmic reticulum | UDP- |
| ester hydrolase 1 | | resident protein 44 | glucose:glycoprotein |
| | | | glucosyltransferase |
| | | | 1 |
| Plasma membrane | Gamma-adducin | Endoplasmin | CD59 |
| calcium-transporting | | | |
| ATPase I | | | |
| Plasma membrane | Gelsolin | ER-Golgi SNARE of 24 | |
| calcium-transporting | | kDa | |
| ATPase 3 | | | |
| Plasma membrane | Kinesin-1 heavy | FACT complex submit | |
| calcium-transporting | chain | SPT16 | |
| ATPase 4 | | | |
| Probable E3 | Microtubule- | Glucosidase 2 subunit beta | |
| ubiquitin-protein | associated protein | | |
| ligase C12orf51 | RP/EB family | | |
| | member 1 | | |
| Rh blood group, | Myosin light | Heme oxygenase 1 | |
| CcEe antigens | chain 4 | | |
| SLC43A3 | Myosin light | Hemogen | |
| | polypeptide 6 | | |
| Sodium/calcium | Myosin, heavy | Heterochromatin protein | |
| exchanger SCL8A3 | chain 11, smooth | 1-binding protein 3 | |
| | muscle | | |
| Sodium/potassium- | Myosin-10 | High mobility group | |
| transporting ATPase | | protein BI | |
| subunit alpha-1 | | | |
| Sodium/potassium- | Myosin-14 | High mobility group | |
| transporting ATPase | | protein B2 | |
| subunit beta-3 | | | |

TABLE 6
Transmembrane and GPI-linked proteins

| | Erythrocyte GPI- |
|---|---|
| Erythrocyte transmembrane proteins | linked proteins |
| Aquaporin 1 | Acetylecholinesterase |
| Cell surface glycoprotein CD44 | CD55 |
| Channel-like integral membrane protein | CD58 |
| Complement receptor 1 | CD59 antigen |
| Erythroblast membrane-associated protein | |
| Glucose transporter glycoprotein | |
| Glycophorin A | |
| Glycophorin A precursor | |
| Glycophorin C isoform 1 | |
| Kell | |
| Membrane protein p55 | |
| Protein band 3 | |
| Rh blood D group antigen polypeptide | |
| Rhesus D category VI type III protein | |
| Similar to glycophorin A | |
| Similar to Lutheran blood group | |
| Solute carrier family 2 (facilitated glucose | |
| transporter) member 1 | |
| Solute carrier family 29 (nucleoside | |
| transporter) member 1 | |

TABLE 7
Erythrocyte intracellular proteins

| | |
|---|---|
| 2′-3′-cyclic-nucleotide 3′-phosphodiesterase | Hypothetical protein XP_100665 |
| Actin alpha and beta chains | Hypothetical protein XP_100925 |
| Adenosine deaminase | Hypothetical protein XP_103707 |
| Adducin alpha subunit | Hypothetical protein XP_106269 |
| Aldolase A | Ig heavy chain V-V region |
| Ankyrin I isoform 2 | ICIAA0340 |
| Ankyrin I isoform 4 | ICIAA1741 protein |
| Ankyrin I splice form 2 | Lyn B protein |
| Arginase type 1 | Phosphatidylinositol-4-phosphate 5 |
| | kinase type III |
| Arginase type 1 erythroid variant | Poly (A)-specific ribonuclease |
| ATP-binding cassette half-transporter | Presenilin-associated protein |
| ATP-binding cassette subfamily C member 6 | Protein band 4.1 |
| bA421H8.2 (novel protein) | Protein band 4.1 (elliptocytosis 1, RH- |
| | linked) |
| B-CAM protein | Protein band 4.2 |
| Block of proliferation 1 | Protein band 4.9 (dematin) |
| C-1-tetrahydrofolate synthase | Protein band 7.2b, stomatin |
| Calcium transporting ATPase 4 | RAB 35 |
| Creatine kinase | Rabphilin-3 A-integrating protein |
| DC 38 | Ral A binding protein |
| Duodenal cytochrome b | RAPIA or RAPIB |
| Enhancer protein | RAP2B |
| Far upstream element binding protein | Similar to adhesive plaque matrix protein |
| | precursor |
| Flotillin 1 | Similar to ankyrin 1 |
| Flotillin 2 47 | Similar to flotillin 2 |
| Glutathione transferase | Similar to RAS-related protein RAB-15 |
| Glyceraldehyde-3-phosphate dehydrogenase | Similar to RAS-related protein RAL-A |
| Hemoglobin alpha | Similar to tropomyosin |
| Hemoglobin beta | Similar to tropomyosin 4 |
| Hemoglobin delta | Spectrin alpha chain |
| Hemoglobin epsilon | Spectrin beta chain |
| Hemoglobin gamma | Translation initiation factor 2C |
| HGTD-P | Tropomodulin |
| Hypothetical protein XP_061743 or XP_089854 | Tropomyosin 3 |
| Hypothetical protein XP_091430 | Tropomyosin isoform |
| Hypothetical protein XP_091724 | Tropomyosinalpha chain (smooth |
| | muscle) 26 |
| Hypothetical protein XP_092517 | Vesicle-associated membrane protein 2 |
| | (synaptobrevin 2) |
| Hypothetical protein XP_095819 | Zona pellucida binding protein |
| Hypothetical protein XP_100510 | |
| Hypothetical protein XP_100619 | |

TABLE 8
Autoimmune diseases and antigens

| Disease | Known antigen |
|---|---|
| Acute rheumatic fever | cross reactive antibodies to cardiac muscle |
| alopecia areata | Trychohyalin, keratin 16 |
| ANCA-associated vasculitis | Neutrophil cytoplasmic antigen, proteinase 3, |
| | myeloperodixase, bacterial permiability |
| | increasing factor |
| autoimmune gastritis | H. K adenosine triphosphatase |
| autoimmune hemolytic | Rh blood group antigens, I antigen |
| anemia | |
| autoimmune hepatitis | nuclear protein, liver-kidney microsome type I. |
| | liver cytosol type I |
| autoimmune myocarditis | cardiac myosin |
| Autoimmune thyroiditis | Thyroid peroxidase, thyroglobulin,, thyroid- |
| | stimulating hormone receptor |
| Autoimmune uveitis | Retinal arrestin (S-antigen) |
| dermatomyositis | Mi2 ATPase |
| diabetes (type 1) | Pancreatic beta cell antigen |
| goodpasture's syndrome | Noncollagenous domain of basement membrane |
| | collagen type IV |
| Graves' disease | Thyroid stimulating hormone receptor |
| Guillain-Barré syndrome | Neurofascin-186, gliomedin, nodal adhesion |
| | molecueles |
| Hypoglycemia | Insulin receptor |
| Idiopathic thrombocytopenic | Platelet integrin GpIIb, GpIIIa |
| purpura | |
| Insulin resistant diabetes | Insulin receptor |
| Membranous nephritis | Phospholipase A2 |
| mixed essential | rheumatoid factor IgG complexes |
| cryoglobulinemia | |
| multiple sclerosis | Myelin basic protein, proteolipid protein, myelin |
| | oligodendrocyte glycoprotein |
| myasthenia gravis | Acetylcholine receptor |
| Myasthenia gravis - MUSC | Muscarinic receptor |
| Pemphigus/pemphigoid | Epidermal cadherin |
| pernicious anemia | intrinsic factor (Gastric) |
| polymyositis | nuclear and nucleolar antigen |
| primary biliary cirrhosis | neutrophil nuclear antigen, mitochondria! |
| | multienzyme complex |
| psoriasis | PSO p27 |
| rheumatoid arthritis | rheumatoid factor IgG complexes, synovial joint |
| | antigen, citrullinated protein, carbamylated |
| | protein |
| scleroderma/systemic | Scl-86, nucleolar scleroderma antigen |
| sclerosis | |
| Sjógren's syndrome | SS-B, Lupus La protein |
| systemic lupus erythematosus | DNA, histones, ribosomes, snRNP, scRNP |
| vitiligo | VIT-90, VIT-75, VIT-40 |
| Wegener's granulomatosis | neutrophil nuclear antigen |
| Anti-phospholipid syndrome | Beta-2 glycoprotein 1 |
| (APS) & catastrophic APS | |
| Chemotherapy induced | Neuronal antigens |
| peripheral neuropathy | |
| Thrombotic | ADAMTSI3 |
| thrombocytopenic purpura | |
| Atypical hemolytic uremic | Complement factor H |
| syndrome | |

TABLE 9
Inflammatory diseases and antigens

| Disease | Antigen |
|---|---|
| Crohn's disease | Flagellin, microbial antigens |
| Ulcerative colitis | Neutrophil cytoplasmic antigen, microbial |
| | antigens |
| Celiac disease | Gluten |
| Inflammatory bowel disease | Microbial antigens |

TABLE 10
Allergic disease triggers

| Allergy | Antigen |
|---|---|
| Animal dander | fel d 1, can f6 |
| Black walnut | 2S albumin, vicilin-like (7S) protein |
| Brazil nut | 2S albumin, legumin-like (11S) seed storage protein |
| Cashew nut | 7S vicilin-like protein, legumin-like 11S seed storage protein |
| Chestnut | Chitinase lb, lipid transfer protein Cas s8 |
| Dust mites | Der p2 |
| Egg | Ovomucoid, ovalbumin, ovotransferrin, lysozyme, alpha-livetin |
| English | 2S albumin, 7S vicilin-like protein, lipid transfer protein, |
| walnut | legumin-like 11S seed storage protein |
| Fish | Parvalbumins |
| Hazelnut | Bet vl homologue, profilin, lipid transfer protein, lls globulin-like |
| | protein, 7S vicilin-like protein |
| Insect venom | Melittin, phospholipase A2, hyaluronidase, acid phosphatase, |
| | protease, antigen 5, Api 1111-4, Bom pl, p4, Dol ml, 2, 5; lisp cl, |
| | c5; Pol al, a2, 115; Ves vl, v2, v5; Sol I 1-4 |
| Latex | Hey b 1, 2, 3, 5, 6.01, 6.02, 8, 9, 11 |
| Milk | Alpha sl casein, beta-lactoglobulin |
| Mold | enzymes, toxins, cell wall components |
| Peanut | Ara hl, Ara h2, Ara h3, Ara h6 |
| Pollen | Aconitate hydratase, fructose bisphosphate aldolase, ATP |
| | synthase, luminal binding protein, calmodulin, calreticulin, |
| | chaperonin, enolase, lipid transfer protein 1, lipid transfer protein |
| | 2, profilins |
| Pollen, grass | Phl p I, 2, 4, 5, 6, 11, 12, 13 |
| Shellfish | arginine kinase, tropomyosin. myosin light chain, sarcoplasmic |
| | calcium binding protein, triose phosphate isomerase, aldolase, |
| | titin |
| Soy | Gly ml soybean hydrophobic protein, gly m4, gly m5, gly m6, |
| | Gly m 2s albumin, lipid transfer proteins, alpha-globulin, |
| Tree nuts | Lipid transfer proteins, profilins, Bet vl-related family, legumins, |
| | vicilins, 2S albumins |
| Wheat | gluten, prolamins, 2S albumins, lipid transer proteins, a- |
| | amylasefprotease inhibitors, puroindoline, alpha-globulin, alpha- |
| | gliadin, beta-gliadin, gamma-gliadin, fast-omega-gliadin, slow- |
| | omega-gliadin |

TABLE 11
Therapeutic protein classes to treat diseases

| | |
|---|---|
| AAV capsid protein | CTLA4 |
| alglocosidase alpha | Erythmpoietin |
| Anti CS | Factor IX |
| Anti gp Iib/IIIa | Factor VII |
| Anti IGE | Factor VIII |
| Anti IL-12, Anti IL-23 | Glatiramer acetate |
| Anti RANK ligand | glucocerebrosidase |
| Anti-alpha 4 integral | GnRH antagonist |
| Anti-APRIL | IgG |
| Anti-BAFF | IL1R antagonist |
| Anti-CD20 | IL1R or IL1 antagonist |
| Anti-CD52 | Insulin |
| Anti-FGPR | Interferon alpha |
| Anti-Her2 | interferon beta |
| Anti-IL2 receptor | Lentivirus capsid protein |
| Anti-IL6 receptor | LFA3-Fc |
| Anti-PD1 | Retrovirus capsid protein |
| Anti-RSV protein F | TACI-Ig |
| Anti-TNFa | TNF-receptor |
| Anti-VEGF | Asparaginase |

TABLE 12
Exogenous antigens
General Classes of Exogenous antigens

| Ankyrin repeat proteins | | Fibronectins | Lyases |
|---|---|---|---|
| Antibodies | Complement receptors | GPI-linkcd | Nanobodies |
| | | polypeptides | |
| Aptamers | Cyclic peptides | HEAT repeat | Nucleic Acids |
| | | proteins | |
| ARM repeat proteins | DARPins | Hydrolases | Polypeptides |
| Carbohydrates | DNAses | Kinases | Single-chain variable |
| | | | fragments (scFv) |
| Cell surface receptors | Enzymes | Lipoproteins | Tetratricopeptide repeat |
| | | | proteins |

Complement-Related Exogenous antigens

| | | | |
|---|---|---|---|
| Cl inhibitor | C4 binding protein | CR3 | Factor I |
| C3 Beta chain Receptor | CD59 | CR4 | Homologous restriction |
| | | | factor |
| C3aR | CR1 | Decay-accelerating | Membrane cofactor |
| | | factor (DAF) | protein (MCP) |
| C3eR | CR2 | Factor H | PRELP |

Enzymes

| | | | |
|---|---|---|---|
| triacylglycerol lipase | bile-acid-CoA | feruloyl esterase | phosphatidate |
| | hydrolase | | phosphatase |
| (S)-methylmalonyl- | bis(2- | formyl-CoA | phosphatidylglycerophos |
| CoA hydrolase | ethylhexyl)phthalate | hydrolase | phatase |
| | esterase | | |
| [acyl-carrier-protein] | bisphosphoglycerate | fructose- | phosphatidylinositol |
| phosphodiesterase | phosphatase | bisphosphatase | deacylase |
| [phosphorylase] | Carboxy lic-Ester | fumarylacetoacetase | phosphodiesterase I |
| phosphatase | Hydrolases | | |
| 1,4-lactonase | carboxymethylenebute | fusarinine-C | phosphoglycerate |
| | nolidase | ormthinesterase | phosphatase |
| 11-cis-retinyl-palmitate | cellulose-polysulfatase | galactolipase | phosphoglycolate |
| hydrolase | | | phosphatase |
| 1-alkyl-2- | cephalosporin-C | gluconolactonase | phosphoinositide |
| acetylglycerophosphocholine | deacetylase | | phospholipase C |
| esterase | | | |
| 2′-hy droxybiphenyl-2- | cerebroside-sulfatase | glucose-1- | phospholipase Al |
| sulfinate desulfinase | | phosphatase | |
| 2-pyrone-4,6- | cetraxate | glucose-6- | phospholipase A2 |
| dicarboxylate lactonase | benzylesterase | phosphatase | |
| 3′,5′-bisphosphate | chlorogenate hydrolase | glutathione | phospholipase C |
| nucleotidase | | thiolesterase | |
| 3-hydroxyisobutyryl- | chlorophyllase | glycerol-1- | phospholipase D |
| CoA hydrolase | | phosphatase | |
| 3′-nucleotidase | cholinesterase | glycerol-2- | phosphonoacetaldehyde |
| | | phosphatase | hydrolase |
| 3-oxoadipate enol- | choline-sulfatase | glycerophosphocholine | phosphonoacetate |
| lactonase | | phosphodiesterase | hydrolase |
| 3-phytase | choloyl-CoA hydrolase | Glycosidases, i.e. | phosphonopyruvate |
| | | enzymes that | hydrolase |
| | | hydrolyse O- and | |
| | | S-glycosyl | |
| | | compounds | |
| 4-hydroxybenzoyl-CoA | chondro-4-stilfatase | glycosulfatase | phosphoprotein |
| thioesterase | | | phosphatase |
| 4-methyloxaloacetate | chondro-6-sulfatase | Glycosylases | Phosphoric-diester |
| esterase | | | hydrolases |
| 4-phytase | citrate-lyase | histidinol- | Phosphoric-monoester |
| | deacetylase | phosphatase | hydrolases |
| 4-pyridoxolactonase | cocaine esterase | hormone-sensitive | Phosphoric-triester |
| | | lipase | hydrolases |
| 5′-nuclemidase | cutinase | Hydrolysing N- | phosphoserine |
| | | glycosyl | phosphatase |
| | | compounds | |
| 6-acetylglucose | cyclamate | Hydrolysing S- | poly(3-hydroxybutyrate) |
| deacetylase | sulfohydrolase | glycosyl | depolymemse |
| | | compounds | |
| 6- | Cysteine | hydroxyacylglutathione | poly(3-hydroxyoctanoate) |
| phosphogluconolactonase | endopeptidases | hydrolase | depolymerase p |
| a-amino-acid esterase | Cysteine-type | hydroxybutyrate- | polyneuridine-aldehyde |
| | carboxypeptidases | dimer hydrolase | esterase |
| a-Amino-acyl-peptide | D-arabinonolactonase | hydroxymethylglut | protein-glutamate |
| hydrolases | | aryl-CoA hydrolase | methylesterase |
| acetoacetyl-CoA | deoxylimonate A-ring- | iduronate-2- | quorum-quenching N- |
| hydrolase | lactonase | sulfatase | acyl-homoserine |
| | | | lactonase |
| acetoxybutynylbithiophene | dGTPase | inositol-phosphate | retinyl-palmitate esterase |
| deacetylase | | phosphatase | |
| acetylajmaline esterase | dihydrocoumarin | juvenile-hormone | Serine dehyrdatase or |
| | hydrolase | esterase | serine hydroxymethyl |
| | | | transferase |
| acetylalkylglycerol | Dipeptidases | kynureninase | Serine endopeptidases |
| acetylhydrolase | | | |
| acetylcholinesterase | Dipeptide hydrolases | L- | serine- |
| | | arabinonolactonase | ethanolluninephosphate |
| | | | phosphodiesterase |
| acetyl-CoA hydrolase | Dipeptidyl-peptidases | limonin-D-ring- | Serine-type |
| | and tripeptidyl- | lactonase | carboxypeptidases |
| | peptidases | | |
| acetylesterase | Diphosphoric- | lipoprotein lipase | S-formylglutathione |
| | monoester hydrolases | | hydrolase |
| acetylpyruvate | disulfoglucosamine-6- | L-rhamnono-1,4- | sialate O-acetylesterase |
| hydrolase | sulfatase | lactonase | |
| acetylsalicylate | Dodecanoyl-[acyl- | lysophospholipase | sinapine esterase |
| deacetylase | carrier-protein] | | |
| | hydrolase | | |
| acetylxylan esterase | Endodeoxyribonucleases | mannitol-1- | Site specific |
| | producing 3′- | phosphatase | endodeoxyribonucleases: |
| | phosphomonoesters | | cleavage is not sequence |
| | | | specific |
| acid phosphatase | Endodeoxyribonucleases | Metallocarboxypeptidases | Site-specific |
| | producing 5′- | | endodeoxyribonucleases |
| | phosphomonoesters | | that are specific for |
| | | | altered bases. |
| Acting on acid | Endopeptidases of | Metalloendopeptidases. | Site-specific |
| anhydrides to catalyse | unknown catalytic | | endodeoxyribonucleases: |
| transmembrane | mechanism | | cleavage is sequence |
| movement of | | | specific |
| substances | | | |
| Acting on acid | Endoribonucleases | methylphosphothio | Sphingomyelin |
| anhydrides to facilitate | producing 3′- | glycerate | phosphodiesterase |
| cellular and subcellular | phosphomonoesters | phosphatase | |
| movement | | | |
| Acting on GTP to | Endoribonucleases | methylumbelliferyl- | S-succinylglutathione |
| facilitate cellular and | producing 5′- | acetate | hydrolase |
| subcellular movement | phosphomonoesters | deacetylase | |
| Acting on phosphorus- | Endoribonucleases that | monoterpene e- | steroid-lactonase |
| nitrogen bonds | are active with either | lactone hydrolase | |
| | ribo- or | | |
| | deoxyribonucleic acids | | |
| | and produce 3′- | | |
| | phosphomonoesters | | |
| Acting on sulfur- | Endoribonucleases that | N- | sterol esterase |
| nitrogen bonds | are active with either | acetylgalactosamine- | |
| | ribo- or | 4-sulfatase | |
| | deoxyribonucleic acids | | |
| | and produce 5′- | | |
| | phosphomonoesters | | |
| actinomvcin lactonase | Enzymes acting on | N- | steryl-sulfatase |
| | acid anhydrides | acetylgalactosamine- | |
| | | 6-sulfatase | |
| acylcamitine hydrolase | Enzymes Acting on | N- | succinyl-CoA hydrolase |
| | carbon-carbon bonds | acetylgalactosamin | |
| | | oglycan | |
| | | deacetylase | |
| acyl-CoA hydrolase | Enzymes acting on | N- | sucrose-phosphate |
| | carbon-nitrogen bonds, | acetylglucosiunine- | phosphatase |
| | other than peptide | 6-sulfatase | |
| | bonds | | |
| acylglycerol lipase | Enzymes acting on | N- | sugar-phosphatase |
| | carbon-phosphorus | sulfoglucosamme | |
| | bonds | sulfohydrolase | |
| acyloxyacyl hydrolase | Enzymes acting on | oleoyl[acyl-[arner- | Sulfuric-ester hydrolases |
| | carbon-sulfur bonds | protein] hydrolase | |
| acylpyruvate hydrolase | Enzymes Acting on | Omega peptidases | tannase |
| | ether bonds | | |
| ADAMTSI3 | Enzymes acting on | orsellinate-depside | Thioester hydmlases |
| | halide bonds | hydrolase | |
| Adenosine deaminase | Enzymes acting on | oxaloacetase | Thioether and |
| | peptide bonds | | trialkylsulfonium |
| | (peptidases) | | hydrolases |
| adenyly1-[glutamate- | Enzymes acting on | palmitoyl[protein] | Threonine endopeptidases |
| ammonia ligase] | phosphorus-nitrogen | hydrolase | |
| hydrolase | bonds | | |
| ADP-dependent | Enzymes acting on | palmitoyl-CoA | thymidine phosphorylase |
| medium-chain-acyl- | sulfur-nitrogen bonds | hydrolase | |
| CoA hydrolase | | | |
| ADP-dependent short- | Enzymes acting on | pectinesterase | trehalose-phosphatase |
| chain-acyl-CoA | sulfur-sulfur bonds | | |
| hydrolase | | | |
| ADP-phosphoglycerate | Ether hydmlases. | Peptidyl peptide | triacetate-lactonase |
| phosphatase | | hydrolases | |
| alkaline phosphatase | Exodeoxyribonucleases | Peptidyl-amino- | Triphosphoric-monoester |
| | producing 5′- | acid hydrolases | hydrolases |
| | phosphomonoesters | | |
| all-trans-retinyl- | Exonucleases that are | Peptidylamino-acid | trithionate hydrolase |
| palmitate hydrolase | active with either ribo- | hydrolases or | |
| | or deoxyribonucleic | acylamino-acid | |
| | acids and produce 3′- | hydrolases | |
| | phosphomonoesters | | |
| aminoacyl-tRNA | Exonucleases that are | Peptidyl- | tropinesterase |
| hydrolase | active with either ribo- | dipeptidases | |
| | or deoxyribonucleic | | |
| | acids and produce 5′- | | |
| | phosphomonoesters | | |
| Aminopeptidases | Exoribonucleases | phenylacetyl-CoA | ubiquitin thiolesterase |
| | producing 3′- | hydrolase | |
| | phosphomonoesters | | |
| arylesterase | Exoribonucleases | Phenylalanine | UDP-sulfoquinovose |
| | producing 5′- | ammonia lyase | synthase |
| | phosphomonoesters. | | |
| arylsulfatase | Factor IX | Phenylalanine | uricase |
| | | hydroxylase | |
| Asparaginase | Factor VIII | pheophorbidase | uronolactonase |
| Aspartic endopeptidases | fatty-acyl-ethyl-ester | phloretin hydrolase | wax-ester hydrolase x |
| | synthase | | |

| | | |
|---|---|---|
| b-diketone hydrolase | phorbol-diester | Xylono-1,4-lactonase |
| | hydrolase | |

Selected Disease, Exogeneous antigens, and Targets

| Category | Disease | Exogenous antigen | Target |
|---|---|---|---|
| Amyloidoses | AA Amyloidosis | an an antibody-like | Serum amyloid A |
| | | binder to serum | protein and amyloid |
| | | amyloid A protein or | placques component |
| | | serum amyloid P | |
| | | component | |
| Amyloidoses | beta2 microglobulin | an an antibody-like | Beta2 microglobulin or |
| | amyloidosis | binder to beta-2 | amyloid placques |
| | | microglobulin or | |
| | | serum amyloid P | |
| | | component | |
| Amyloidoses | Light chain | an an antibody-like | Antibody light chain or |
| | amyloidosis | hinder to light chain, | amyloid placques |
| | | serum amyloid P | |
| | | component | |
| Cell clearance | Cancer | an an antibody-like | a circulating tumor cell |
| | | binder to CD44 | |
| Cell clearance | Cancer | an an antibody-like | a circulating tumor cell |
| | | binder to EpCam | |
| Cell clearance | Cancer | an an antibody-like | a circulating tumor cell |
| | | binder to Hcr2 | |
| Cell clearance | Cancer | an an antibody-like | a circulating tumor cell |
| | | binder to EGFR | |
| Cell clearance | Cancer (6 cell) | an an antibody-like | a cancerous B cell |
| | | binder to CD20 | |
| Cell clearance | Cancer .6 cell) | an an antibody-like | a cancerous B cell |
| | | binder to CDI9 | |
| Clearance Ab | Antiphospholipid | beta2-glycoprotein-1 | pathogenic self- |
| | syndrome | | antibody against beta2- |
| | | | glycoprotein-I |
| Clearance Ab | Catastrophic | beta2-glycoprotein-1 | pathogenic self- |
| | antiphospholipid | | antibody against beta2- |
| | syndrome | | glycoprotein-I |
| Clearance Ab | Cold agglutinin disease | I/i antigen | Pathogenic self- |
| | | | antibody against I/i |
| | | | antigen |
| Clearance Ab | Goodpasture syndrome | a3 NCI domain of | pathogenic self- |
| | | collagen (IV) | antibody against a3 |
| | | | NCI domain of |
| | | | Collagen (IV) |
| Clearance Ab | Immune | Platelet Glycoproteins | pathogenic self- |
| | thrombocytopenia | (Ib-IX, IIb-IIIa, IV, Ia- | antibody against |
| | purpura | IIa) | platelet glycoprotein |
| Clearance Ab | Membranous | Phospholipase A2 | pathogenic self- |
| | Nephropathy | receptor | antibody against |
| | | | phospholipase A2 |
| | | | receptor |
| Clearance Ab | Warm antibody | Glycophorin A, | pathogenic self- |
| | hemolytic anemia | glycophorin B, and/or | antibody against |
| | | glycophorin C, Rh | glycophorins and/or Rh |
| | | antigen | antigen |
| Complement | Age-related macular | a suitable complement | active complement |
| | degeneration | regulatory protein | |
| Complement | Atypical hemolytic | complement factor H, | active complement |
| | uremic syndrome | or a suitable | |
| | | complement regulatory | |
| | | protein | |
| Complement | Autoimmune | a suitable complement | active complement |
| | hemolytic anemia | regulatory molecule | |
| Complement | Complement Factor I | Complement factor 1, | active complement |
| | deficiency | a suitable complement | |
| | | regulatory protein | |
| Complement | Non-alcoholic | a suitable complement | active complement |
| | steatohepatitis | regulatory molecule | |
| Complement | Paroxysmal nocturnal | a suitable complement | active complement |
| | hemoglobinuria | regulatory protein | |
| Enzyme | 3-methylcrotonyl-CoA | 3 -methylcrotonyl-CoA | 3-hydroxyvalerylcamitine, |
| | carboxylase deficiency | carboxylase | 3-methylcrotonylglycine |
| | | | (3-MCG) and 3- |
| | | | hydroxyisovaleric acid |
| | | | (3- HIVA) |
| Enzyme | Acute Intermittent | Porphobilinogen | Porphobilinogen |
| | Porphyria | deaminase | |
| Enzyme | Acute lymphoblastic | Asparaginase | Asparagine |
| | leukemia | | |
| Enzyme | Acute lymphocytic | Asparaginase | Asparagine |
| | leukemia, acute | | |
| | myeloid leukemia | | |
| Enzyme | Acute myeloblastic | Asparaginase | Asparagine |
| | leukemia | | |
| Enzyme | Adenine | adenine | Insoluble purine 2,8- |
| | phosphoribosyltransferase | phosphorihosyltnansfemse | dihydroxyadenine |
| | deficiency | | |
| Enzyme | Adenosine deaminase | Adenosine deaminase | Adenosine |
| | deficiency | | |
| Enzyme | Afibrinogenomia | FI | enzyme replacement |
| Enzyme | Alcohol poisoning | Alcohol | Ethanol |
| | | dehydrogenase/oxidase | |
| Enzyme | Alexander's disease | FVII | enzyme replacement |
| Enzyme | Alkaptonuria | homogentisate oxidase | homogentisate |
| Enzyme | Argininemia | Ammonia | ammonia |
| | | monooxygenase | |
| Enzyme | argininosuccinate | Ammonia | ammonia |
| | aciduria | monooxygenase | |
| Enzyme | citrullinemia type I | Ammonia | ammonia |
| | | monooxygenase | |
| Enzyme | Citrullinemia type II | Ammonia | ammonia |
| | | monooxygenase | |
| Enzyme | Complete LCAT | Lecithin-cholesterol | Cholesterol |
| | deficiency, Fish-eye | acyltransferase | |
| | disease, | (LCAT) | |
| | atherosclerosis, | | |
| | hypercholesterolemia | | |
| Enzyme | Cyanide poisoning | Thiosulfate-cyanide | Cyanide |
| | | sulfurtransferase | |
| Enzyme | Diabetes | Hexokirtase, | Glucose |
| | | glucokinase | |
| Enzyme | Factor II Deficiency | FII | enzyme replacement |
| Enzyme | Familial | Arginase | Arginine |
| | hyperarginemia | | |
| Enzyme | Fibrin Stabilizing | FXIII | enzyme replacement |
| | factor Def. | | |
| Enzyme | Glutaric acidemia type | lysine oxidase | 3-hydroxyglutaric and |
| | I | | glutaric acid (C5-DC), |
| | | | lysine |
| Enzyme | Gout | Uricase | Uric Acid |
| Enzyme | Gout - hyperuricemia | Uricase | Uric acid (Urate |
| | | | crystals) |
| Enzyme | Hageman Def. | FXII | enzyme replacement |
| Enzyme | Hemolytic anemia due | pyrimidine 5′ | pyrimidines |
| | to pyrimidine 5′ | nucleotidase | |
| | nucleotidase deficiency | | |
| Enzyme | Hemophilia A | Factor VIII | Thrombin (factor II a) |
| | | | or Factor X |
| Enzyme | Hemophilia B | Factor DC | Factor XIa or Factor X |
| Enzyme | Hemophilia C | FXI | enzyme replacement |
| Enzyme | Hepatocellular | Arginine deiminase | Arginine |
| | carcinoma, melanoma | | |
| Enzyme | Homocystinuria | Cystathionine B | homocysteine |
| | | synthase | |
| Enzyme | hyperammonemia/ | Ammonia | Ammonia |
| | ornithinemia/citrullinemia | monooxygenase | |
| | (ornithine transporter | | |
| | defect) | | |
| Enzyme | Isovaleric acidemia | Leucine metabolizing | leucine |
| | | enzyme | |
| Enzyme | Lead poisoning | d-aminolevulinate | lead |
| | | dehydrogenase | |
| Enzyme | Lesch-Nyhan | Uricase | Uric acid |
| | syndrome | | |
| Enzyme | Maple syrup urine | Leucine metabolizing | Leucine |
| | disease | enzyme | |
| Enzyme | Methylmalonic | methylmalonyl-CoA | methylmalonate |
| | acidemia (vitamin b | mutase | |
| | 12 non-responsive) | | |
| Enzyme | Mitochondrial | thymidine | thymidine |
| | neurogastrointestinal | phosphorylase | |
| | encephalomyopathy | | |
| Enzyme | Mitochondrial | Thymidine | Thymidine |
| | neurogastrointestinal | phosphorylase | |
| | encephalomyopathy | | |
| | (MNGIE) | | |
| Enzyme | Owren's disease | FV | enzyme replacement |
| Enzyme | p53-mill solid tumor | Serine dehyrdatase or | serine |
| | | serine hydroxymethyl | |
| | | transferase | |
| Enzyme | Pancreatic | Asparaginase | asparagine |
| | adenocarcinoma | | |
| Enzyme | Phenylketonuria | Phenylalanine | Phenylalanine |
| | | hydroxylase, | |
| | | phenylalanine | |
| | | ammonia lyase | |
| Enzyme | Primary hyperoxaluria | Oxalate oxidase | Oxalate |
| Enzyme | Propionic acidemia | Propionate conversion | Proprionyl coA |
| | | enzyme? | |
| Enzyme | Purine nucleoside | Purine nucleoside | Inosine, dGTP |
| | phosphorylase | phosphorylase | |
| | deficiency | | |
| Enzyme | Stuart-Power Def. | FX | enzyme replacement |
| Enzyme | Thrombotic | ADAMTS 13 | ultra-large von |
| | Thrombocytopenic | | willebrand factor |
| | Purpura | | (ULVWF) |
| Enzyme | Transferase deficient | galactose | Galactose-I -phosphate |
| | galactosemia | dehydrogenase | |
| | (Galactosemia type 1) | | |
| Enzyme | Tyrosinemia type 1 | tyrosine phenol-lyase | tyrosine |
| Enzyme | von Willebrand disease | vWF | enzyme replacement |
| IC clearance | IgA Nephropathy | Complement receptor I | Immune complexes |
| IC clearance | Lupus nephritis | Complement receptor | immune complex |
| | | 1 | |
| IC clearance | Systemic lupus | Complement receptor | immune complex |
| | erythematosus | 1 | |
| Infections | Anthrax (R. anthraris) | an an antibody-like | B. anthraris |
| | infection | binder to B. anthracis | |
| | | surface protein | |
| Infectious | C. botulinum infection | an an antibody-like | C. botilinum |
| | | binder to C. botulinum | |
| | | surface protein | |
| Infectious | C. difficile infection | an antibody-like binder | C. dificile |
| | | to C. difficile surface | |
| | | protein | |
| Infectious | Candida infection | an antibody-like binder | candida |
| | | to candida surface | |
| | | protein | |
| Infectious | E. coli infection | an antibody-like binder | E. coli |
| | | to E. coli surface | |
| | | protein | |
| Infectious | Ebola infection | an antibody-like binder | Ebola |
| | | to Ebola surface | |
| | | protein | |
| Infectious | Hepatitis B (HBV) | an antibody-like binder | HBV |
| | infection | to HBV surface protein | |
| Infectious | Hepatitis C (HCV) | an antibody-like binder | HCV |
| | infection | to HCV surface protein | |
| Infectious | Human | an antibody-like binder | HIV |
| | immunodeficiency | to HIV envelope | |
| | virus (HIV) infection | proteins or CD4 or | |
| | | CCRS or | |
| Infectious | M. tuberculosis | an antibody-like binder | M. tuberculosis |
| | infection | to M. tuberculosis | |
| | | surface protein | |
| Infections | Malaria (P falciparum) | an antibody-like hinder | P falciparum |
| | infection | to P. falriparutm | |
| | | surface protein | |
| Lipid | Hepatic lipase | Hepatic lipase (LIPC) | Lipoprotein, |
| | deficiency, | | intermediate density |
| | hypercholesterolemia | | (IDL) |
| Lipid | Hyperalphalipoproteinemia | Cholesteryl ester | Lipoprotein, high |
| | I | transfer protein(CETP) | density (HDL) |
| Lipid | hypercholesterolemia | an antibody-like binder | LDL |
| | | to low-density | |
| | | lipoprotein (LDL), | |
| | | LDL receptor | |
| Lipid | hypercholesterolemia | an antibody-like binder | HDL |
| | | to high-density | |
| | | lipoprotein (HDL) or | |
| | | HDL receptor | |
| Lipid | lipoprotein lipase | lipoprotein lipase | chilomicrons and very |
| | deficiency | | low density |
| | | | lipoproteins (VLDL) |
| Lipid | Lipoprotein lipase | lipoprotein lipase | Lipoprotein, very low |
| | deficiency, disorders | (LPL) | density (VLDL) |
| | of lipoprotein | | |
| | metabolism | | |
| Lysosomal storage | Aspartylglucosaminuria | N- | glycoproteins |
| | (208400) | Aspartylglucosaminidase | |
| Lysosomal storage | Cerebrotendinous | Sterol 27-hydroxylase | lipids, cholesterol, and |
| | xanthomatosis | | bile acid |
| | (cholestanol lipidosis; | | |
| | 213700) | | |
| Lysosomal storage | Ceroid lipofuscinosis | Palmitoyl-protein | lipopigments |
| | Adult form (CLN4, | thioesterase-1 | |
| | Kufs' disease; 204300) | | |
| Lysosomal storage | Ceroid lipofuscinosis | Paliiiitoyl-protein | lipopigments |
| | Infantile form (CLN1, | thioesterase-1 | |
| | Santavuon-Haltia | | |
| | disease; 256730) | | |
| Lysosomal storage | Ceroid lipofuscinosis | Lysosomal | lipopigments |
| | Juvenile form (CLN3, | transmembrane CLN3 | |
| | Batten disease, Vogt- | protein | |
| | Spielmeyer disease; | | |
| | 204200) | | |
| Lysosomal storage | Ceroid lipofuscinosis | Lysosomal pepstatin- | lipopigments |
| | Late infantile form | insensitive peptidase | |
| | (CLN2, JansIcy- | | |
| | Bielschowslcy disease; | | |
| | 204500) | | |
| Lysosomal storage | Ceroid lipofuscinosis | Transmembrane CLN8 | lipopigments |
| | Progressive epilepsy | protein | |
| | with intellectual | | |
| | disability (600143) | | |
| Lysosomal storage | Ceroid lipofuscinosis | Transmembrane CLN6 | lipopigments |
| | Variant late infantile | protein | |
| | form (CT N6; 601780) | | |
| Lysosomal storage | Ceroid lipofuscinosis | Lysosomal | lipopigments |
| | Variant late infantile | transmembrane CLN5 | |
| | form, Finnish type | protein | |
| | (CLNS; 256731) | | |
| Lysosomal storage | Cholesteryl ester | lysosomal acid lipase | lipids and cholesterol |
| | stomge disease | | |
| | (CESD) | | |
| Lysosomal storage | Congenital disorders of | Phosphomannomutase- | N-glycosylated protein |
| | N-glycosylation CDC | 2 | |
| | la (solely neurologic | | |
| | and neurologic- | | |
| | multivisceral forms; | | |
| | 212065) | | |
| Lysosomal storage | Congenital disorders of | Mannose (Man) | N-glycosylated protein |
| | N-glycosylation CDG | phosphate (P) | |
| | lb (602579) | isomerase | |
| Lysosomal storage | Congenital disorders of | Dolicho-P-GIc: | N-glycosylated protein |
| | N-glycosylation CDG | Man9GIcNAc2-PP- | |
| | Ic (603147) | dolichol | |
| | | glucosyltransferase | |
| Lysosomal storage | Congenital disorders of | Dolicho-P-Man: | N-glycosylated protein |
| | N-glycosylation CDG | Man5GIcNAc2-PP- | |
| | Id (601110) | dolichol | |
| | | mannosyltransferase | |
| Lysosomal storage | Congenital disorders of | Dolichol-P-mannose | N-glycosylated protein |
| | N-glycosylation CDG | synthase | |
| | Ie (608799) | | |
| Lysosomal storage | Congenital disorders of | Protein involved in | N-glycosylated protein |
| | N-glycosylation CDG | mannose-P-dolichol | |
| | If (609180) | utilization | |
| Lysosomal storage | Congenital disorders of | Dolichyl-P-mannose; | N-glycosylated protein |
| | N-glycosylation CDG | Man-7-GIcNAc-2-PP- | |
| | Ig (607143) | dolichyl-\acute{α}-6- | |
| | | mannosyltransferase | |
| Lysosomal storage | Congenital disorders of | Dolichyl-P-glucose: G | N-glycosylated protein |
| | N-glycosylation CDG | lc-1-Man-9-01cNAc- | |
| | lh (608104) | 2-PP-dolichyl-a-3- | |
| | | glucosyltransferase | |
| Lysosomal storage | Congenital disorders of | \acute{α}-1,3- | N-glycosylated protein |
| | N-glycosylation CDG | Mannosyltransferase | |
| | Ii (607906) | | |
| Lysosomal storage | Congenital disorders of | Mannosyl-\acute{α}-1,6- | N-glycosylated protein |
| | N-glycosylation CDG | glycoprotein-β-1, 2-N- | |
| | Ea (212066) | acetylglucosminyltransferase | |
| Lysosomal storage | Congenital disorders of | Glucosidase I | N-glycosylated protein |
| | N-glycosylation CDG | | |
| | fib (606056) | | |
| Lysosomal storage | Congenital disorders of | GDP-fucose | N-glycosylated protein |
| | N-glycosylation CDG | transporter-I | |
| | tic (Rambam-Hasharon | | |
| | syndrome; 266265 | | |
| Lysosomal storage | Congenital disorders of | β-1,4- | N-glycosylated protein |
| | N-glycosylation CDG | Galactosyltransferase | |
| | Ed (607091) | | |
| Lysosomal storage | Congenital disorders of | Oligomeric Golgi | N-glycosylated protein |
| | N-glycosylation CDG | complex-7 | |
| | De (608779) | | |
| Lysosomal storage | Congenital disorders of | UDP-GIcNAc: | N-glycosylated protein |
| | N-glycosylation CDG | dolichyl-P NAcGIc | |
| | Ij (608093) | phosphotransferase | |
| Lysosomal storage | Congenital disorders of | β-1, | N-glycosylated protein |
| | N-glycosylation CDG | 4Mannosyltransferase | |
| | Tk (608540) | | |
| Lysosomal storage | Congenital disorders of | \acute{α}-1,2- | N-glycosylated protein |
| | N-glycosylation CDG | Mannosyltransferase | |
| | II (608776) | | |
| Lysosomal storage | Congenital disorders of | \acute{α}-1,2- | N-glycosylated protein |
| | N-glycosylation, type I | Mannosyltransferase | |
| | (pre-Golgi | | |
| | glycosylation defects) | | |
| Lysosomal storage | Cystinosis | Cystinosin (lysosomal | Cysteine |
| | | cystine transporter) | |
| Lysosomal storage | Fabry's disease | Trihexosylceramide a- | globotriaosylceramide |
| | (301500) | galactosidase | |
| Lysosomal storage | Farber's disease | Ceramidase | lipids |
| | (lipogmnulomatosis; | | |
| | 228000) | | |
| Lysosomal storage | Fucosidosis (230000) | \acute{α}-L-Fucosidase | fucose and complex |
| | | | sugars |
| Lysosomal storage | Galactosialidosis | Protective | lysosomal content |
| | (Goldberg's syndrome, | protein/cathepsin A | |
| | combined | (PPCA) | |
| | neuraminidase and (β- | | |
| | galactosidase | | |
| | deficiency; 256540) | | |
| Lysosomal storage | Gaucher's disease | Glucosylcerainide β- | sphingolipids |
| | | glucosidase | |
| Lysosomal storage | Glutamyl ribose-5- | ADP-ribose protein | glutamyl ribose 5- |
| | phosphate storage | hydrolase | phosphate |
| | disease (305920) | | |
| Lysosomal storage | Glycogen storage | alpha glucosidase | glycogen |
| | disease type 2 | | |
| | (Pompe's disease) | | |
| Lysosomal storage | GM1 gangliosidosis, | Ganglioside β- | acidic lipid material, |
| | generalized | galactosidase | gangliosides |
| Lysosomal storage | GM2 activator protein | GM2 activator protein | gangliosides |
| | deficiency (Tay-Sachs | | |
| | disease AB variant, | | |
| | GM2A; 272750) | | |
| Lysosomal storage | GM2 gangliosidosis | Ganglioside β- | gangliosides |
| | | galactosidase | |
| Lysosomal storage | Infantile sialic acid | Na phosphate | sialic acid |
| | storage disorder | cotransporter, sialin | |
| | (269920) | | |
| Lysosomal storage | Krabbe's disease | Galactosylceramide β- | sphingolipids |
| | (245200) | galactosidase | |
| Lysosomal storage | Lysosomal acid lipase | Lysosomal acid lipase | cholestery esters and |
| | deficiency (278000) | | triglycerides |
| Lysosomal storage | Metachromatic | Arylsul fatase A | sulfatides |
| | leukodystrophy | | |
| | (250100) | | |
| Lysosomal storage | Mucolipidosis ML II | N- | N-linked glycoproteins |
| | (I-cell disease; | Acetylglucosaminyl-1- | |
| | 252500) | phosphotransfeerase | |
| | | catalytic subunit | |
| Lysosomal storage | Mucolipidosis ML III | N-acetylglucosaminyl- | N-linked glycoproteins |
| | (pseudo-Hurler's | 1-phosphotransfeerase | |
| | polydystrophy) | | |
| Lysosomal storage | Mucolipidosis ML III | Catalytic subunit | N-linked glycoproteins |
| | (pseudo-Hurler's | | |
| | polydystrophy) Type | | |
| | 111-A (252600) | | |
| Lysosomal storage | Mucolipidosis ML III | Substrate-recognition | N-linked glycoproteins |
| | (pseudo-Hurler's | subunit | |
| | polydystrophy) Type | | |
| | HI-C (252605) | | |
| Lysosomal storage | Mucopolysaccharidosis | \acute{α}-l-lduronidase | glycosaminoglycans |
| | MPS I HIS (Hurler- | | |
| | Scheie syndrome; | | |
| | 607015) | | |
| Lysosomal storage | Mucopolysaccharidosis | \acute{α}-I-Iduronidase | glycosaminoglycans |
| | MPS I-H (Hurler's | | |
| | syndrome; 607014) | | |
| Lysosomal storage | Mucopolysaccharidosis | Iduronate sulfate | glycosaminoglycans |
| | MPS 11 (Hunter's | sulfatase | |
| | syndrome; 309900) | | |
| Lysosomal storage | Mucopolysaccharidosis | Heparan-S-sulfate | glycosaminoglycans |
| | MPS HI | sulfamidase | |
| | (Sanfilippo's | | |
| | syndrome) Type HI-A | | |
| | (252900) | | |
| Lysosomal storage | Mucopolysaccharidosis | N-acetyl-D- | glycosaminoglycans |
| | MPS HI | glucosaminidase | |
| | (Sanfilippo's | | |
| | syndrome) Type HI-B | | |
| | (252920) | | |
| Lysosomal storage | Mucopolysaccharidosis | Acetyl-CoA- | glycosaminoglycans |
| | MPS HI | glucosaminide N- | |
| | (Sanfilippo's | acetyltranaferase | |
| | syndrome) Type HI-C | | |
| | (252930) | | |
| Lysosomal storage | Mucopolysaccharidosis | N-acetyl- | glycosaminoglycans |
| | MPS HI | glucosaminine-6- | |
| | (Sanfilippo's | sulfate sulfatase | |
| | syndrome) Type HI-D | | |
| | (252940) | | |
| Lysosomal storage | Mucopolysaccharidosis | \acute{α}-I-Iduronidase | glycosaminoglycans |
| | MPS I-S (Seheie's | | |
| | syndrome; 607016) | | |
| Lysosomal storage | Mucopolysaccharidosis | Galactosamine-6- | glycosaminoglycans |
| | MPS IV (Morquio's | sulfate sulfatase | |
| | syndrome) Type IV-A | | |
| | (253000) | | |
| Lysosomal storage | Mucopolysaccharidosis | β-Galactosidase | glycosaminoglycans |
| | MPS IV (Morquio's | | |
| | syndrome) Type IV-B | | |
| | (253010) | | |
| Lysosomal storage | Mucopolysaccharidosis | Hyaltutwidase | glycosaminoglycans |
| | MPS IX | deficiency | |
| | (hyaluronidase | | |
| | deficiency; 601492) | | |
| Lysosomal storage | Mucopolysaccharidosis | N-Acetyl | glycosaminoglycans |
| | MPS VI (Maroteaux- | galactosamine \acute{α}-4- | |
| | Lamy syndrome; | sulfate sulfatase | |
| | 253200) | (aiylsulfatase B) | |
| Lysosomal storage | Mucopolysaccharidosis | β-Glucuronidase | glycosaminoglycans |
| | MPS VII (Sly's | | |
| | syndrome; 253220) | | |
| Lysosomal storage | Mucosulfatidosis | Sulfatase-modifying | sulfatides |
| | (multiple sulfatase | factor-1 | |
| | deficiency; 272200) | | |
| Lysosomal storage | Niemann-Pick disease | Sphingomyelinase | sphingomyelin |
| | type A | | |
| Lysosomal storage | Niemann-Pick disease | Sphingomyelinase | sphingomyelin |
| | type B | | |
| Lysosomal storage | Niemann-Pick disease | NPCI protein | sphingomyelin |
| | Type Cl/Type 13 | | |
| | ((257220) | | |
| Lysosomal storage | Niemann-Pick disease | Epididymal secretory | sphingomyelin |
| | Type C2 (607625) | protein 1 (HE1; NPC2 | |
| | | protein) | |
| Lysosomal storage | Prosaposin deficiency | Prosaposin | sphingolipids |
| | (176801) | | |
| Lysosomal storage | Pycnodysostosis | Cathepsin K | kinins |
| | (265800) | | |
| Lysosomal storage | Sandhofrs disease; | β-Hocosaminidase B | gangliosides |
| | 268800 | | |
| Lysosomal storage | Saposin B deficiency | Saposin B | sphingolipids |
| | (sulfatide activator | | |
| | deficiency) | | |
| Lysosomal storage | Saposin C deficiency | Saposin C | sphingolipids |
| | (Gaucher's activator | | |
| | deficiency) | | |
| Lysosomal storage | Schindler's disease | N-Acetyl- | glycoproteins g |
| | Type I (infantile severe | galactostuninidase | |
| | foray 609241) | | |
| Lysosomal storage | Schindler's disease | N-Acetyl- | lycoproteins |
| | Type II (Kanzaki | galactosaminidase | |
| | disease, adult-onset | | |
| | form; 609242) | | |
| Lysosomal storage | Schindler's disease | N-Acetyl- | glycoproteins |
| | Type HI (intermediate | galactosaminidase | |
| | form; 609241) | | |
| Lysosomal storage | Sialidosis (256550) | Neuriuninidase 1 | mucopolysaccharides |
| | | (sialidase) | and mucolipids |
| Lysosomal storage | Sialuria Finnish type | Na phosphate | sialic acid |
| | (Salta disease; 604369) | cotransporter, sialin | |
| Lysosomal storage | Sialuria French type | UDP-N- | sialic acid |
| | (269921) | acetylglucosamine-2- | |
| | | epimerase/N- | |
| | | acetylmannosamine | |
| | | kinase, sialin | |
| Lysosomal storage | Sphingolipidosis Type | Ganglioside β- | sphingolipids |
| | I (230500) | galactosidase | |
| Lysosomal storage | Sphingolipidosis Type | Ganglioside β- | sphingolipids |
| | II (juvenile type; | galactosidase | |
| | 230600) | | |
| Lysosomal storage | Sphingolipidosis Type | Ganglioside β- | sphingolipids |
| | III (adult type; | galactosidase | |
| | 230650) | | |
| Lysosomal storage | Tay-Sachs disease; | β-Hexosaminidase A | gangliosides |
| | 272800 | | |
| Lysosomal storage | Winchester syndrome | Metal loproteinase- 2 | mucopolysaccharides |
| | (277950) | | |
| Lysosomal storage | Wolman's disease | lysosomal acid lipase | lipids and cholesterol |
| Lysosomal storage | \acute{α}-Mannosidosis | \acute{α}-D-Mannosidase | carbohydrates and |
| | (248500). type I | | glycoproteins |
| | (severe) or II (mild) | | |
| Lysosomal storage | β-Mannosidosis | β-D-Mannosidase | carbohydrates and |
| | (248510) | | glycoproteins |
| Toxic Molecule | alpha hemolysin | an antibody-like binder | alpha hemolysin |
| | poisoning | to alpha hemolysin | |
| Toxic Molecule | antrax toxin poisoning | an antibody-like binder | anthrax toxin |
| | | to anthrax toxin | |
| Toxic Molecule | bacterial toxin-induced | an antibody-like binder | bacterial toxin |
| | shock | to bacterial toxin | |
| Toxic Molecule | botulinum toxin | an antibody-like binder | botulinum toxin |
| | poisoning | to botulinum toxin | |
| Toxic Molecule | Hemochromatosis | iron chelator | molecular iron |
| | (iron poisoning) | | |
| Toxic Molecule | Methanol poisoning | Methanol | Methanol |
| | | dehdrogenase | |
| Toxic Molecule | Nerve gas poisoning | Butyryl cholinesterase | Sarin |
| Toxic Molecule | Prion disease caused | an antibody-like binder | Prion protein PRP |
| | by PRP | to prion protein PRP | |
| Toxic Molecule | Prion disease caused | an antibody-like binder | Prion protein PRPc |
| | by PRPc | to prion protein PRPc | |
| Toxic Molecule | Prion disease caused | an antibody-like binder | Prion protein PRPsc |
| | by | to prion protein PRPsc | |
| Toxic Molecule | PRPsc Prion disease | an antibody-like binder | Prion protein PRPres |
| | cussed by PRPrcs | to prion protein | |
| | | PRPres | |
| Toxic Molecule | Sepsis or cytokine | an antibody-like binder | cytokines |
| | storm | to cytokines or Duffy | |
| | | antigen receptor of | |
| | | chemokines (DARC) | |
| Toxic Molecule | spider venom | an antibody-like binder | spider venom |
| | poisoning | to spider venom | |
| Toxic Molecule | Wilson disease | copper chelator | molecular copper |

TABLE 13
Selected Disease, Exogeneous antigens, and Targets

| Category | Disease | Exogenous antigen | Target |
|---|---|---|---|
| Amyloidoses | AA Amyloidosis | an an antibody-like | Serum amyloid A |
| | | binder to serum | protein and amyloid |
| | | amyloid A protein or | placques component |
| | | serum amyloid P | |
| | | component | |
| Amyloidoses | beta2 microglobulin | an an antibody-like | Beta2 microglobulin or |
| | amyloidosis | binder to beta-2 | amyloid placques |
| | | microglobulin or | |
| | | serum amyloid P | |
| | | component | |
| Amyloidoses | Light chain | an an antibody-like | Antibody light chain or |
| | amyloidosis | hinder to light chain, | amyloid placques |
| | | serum amyloid P | |
| | | component | |
| Cell clearance | Cancer | an an antibody-like | a circulating tumor cell |
| | | binder to CD44 | |
| Cell clearance | Cancer | an an antibody-like | a circulating tumor cell |
| | | binder to EpCam | |
| Cell clearance | Cancer | an an antibody-like | a circulating tumor cell |
| | | binder to Hcr2 | |
| Cell clearance | Cancer | an an antibody-like | a circulating tumor cell |
| | | binder to EGFR | |
| Cell clearance | Cancer (6 cell) | an an antibody-like | a cancerous B cell |
| | | binder to CD20 | |
| Cell clearance | Cancer .6 cell) | an an antibody-like | a cancerous B cell |
| | | binder to CDI9 | |
| Clearance Ab | Antiphospholipid | beta2-glycoprotein-1 | pathogenic self- |
| | syndrome | | antibody against beta2- |
| | | | glycoprotein-I |
| Clearance Ab | Catastrophic | beta2-glycoprotein-1 | pathogenic self- |
| | antiphospholipid | | antibody against beta2- |
| | syndrome | | glycoprotein-I |
| Clearance Ab | Cold agglutinin disease | I/i antigen | Pathogenic self- |
| | | | antibody against I/i |
| | | | antigen |
| Clearance Ab | Goodpasture syndrome | a3 NCI domain of | pathogenic self- |
| | | collagen (IV) | antibody against a3 |
| | | | NCI domain of |
| | | | Collagen (IV) |
| Clearance Ab | Immune | Platelet Glycoproteins | pathogenic self- |
| | thrombocytopenia | (Ib-IX, IIb-IIIa, IV, Ia- | antibody against |
| | purpura | IIa) | platelet glycoprotein |
| Clearance Ab | Membranous | Phospholipase A2 | pathogenic self- |
| | Nephropathy | receptor | antibody against |
| | | | phospholipase A2 |
| | | | receptor |
| Clearance Ab | Warm antibody | Glycophorin A, | pathogenic self- |
| | hemolytic anemia | glycophorin B, and/or | antibody against |
| | | glycophorin C, Rh | glycophorins and/or Rh |
| | | antigen | antigen |
| Complement | Age-related macular | a suitable complement | active complement |
| | degeneration | regulatory protein | |
| Complement | Atypical hemolytic | complement factor H, | active complement |
| | uremic syndrome | or a suitable | |
| | | complement regulatory | |
| | | protein | |
| Complement | Autoimmune | a suitable complement | active complement |
| | hemolytic anemia | regulatory molecule | |
| Complement | Complement Factor I | Complement factor 1, | active complement |
| | deficiency | a suitable complement | |
| | | regulatory protein | |
| Complement | Non-alcoholic | a suitable complement | active complement |
| | steatohepatitis | regulatory molecule | |
| Complement | Paroxysmal nocturnal | a suitable complement | active complement |
| | hemoglobinuria | regulatory protein | |
| Enzyme | 3-methylcrotonyl-CoA | 3 -methylcrotonyl-CoA | 3-hydroxyvalerylcamitine, |
| | carboxylase deficiency | carboxylase | 3-methylcrotonylglycine |
| | | | (3-MCG) and 3- |
| | | | hydroxyisovaleric acid |
| | | | (3- HIVA) |
| Enzyme | Acute Intermittent | Porphobilinogen | Porphobilinogen |
| | Porphyria | deaminase | |
| Enzyme | Acute lymphoblastic | Asparaginase | Asparagine |
| | leukemia | | |
| Enzyme | Acute lymphocytic | Asparaginase | Asparagine |
| | leukemia, acute | | |
| | myeloid leukemia | | |
| Enzyme | Acute myeloblastic | Asparaginase | Asparagine |
| | leukemia | | |
| Enzyme | Adenine | adenine | Insoluble purine 2,8- |
| | phosphoribosyltransferase | phosphorihosyltnansfemse | dihydroxyadenine |
| | deficiency | | |
| Enzyme | Adenosine deaminase | Adenosine deaminase | Adenosine |
| | deficiency | | |
| Enzyme | Afibrinogenomia | FI | enzyme replacement |
| Enzyme | Alcohol poisoning | Alcohol | Ethanol |
| | | dehydrogenase/oxidase | |
| Enzyme | Alexander's disease | FVII | enzyme replacement |
| Enzyme | Alkaptonuria | homogentisate oxidase | homogentisate |
| Enzyme | Argininemia | Ammonia | ammonia |
| | | monooxygenase | |
| Enzyme | argininosuccinate | Ammonia | ammonia |
| | aciduria | monooxygenase | |
| Enzyme | citrullinemia type I | Ammonia | ammonia |
| | | monooxygenase | |
| Enzyme | Citrullinemia type II | Ammonia | ammonia |
| | | monooxygenase | |
| Enzyme | Complete LCAT | Lecithin-cholesterol | Cholesterol |
| | deficiency, Fish-eye | acyltransferase | |
| | disease, | (LCAT) | |
| | atherosclerosis, | | |
| | hypercholesterolemia | | |
| Enzyme | Cyanide poisoning | Thiosulfate-cyanide | Cyanide |
| | | sulfurtransferase | |
| Enzyme | Diabetes | Hexokirtase, | Glucose |
| | | glucokinase | |
| Enzyme | Factor II Deficiency | FII | enzyme replacement |
| Enzyme | Familial | Arginase | Arginine |
| | hyperarginemia | | |
| Enzyme | Fibrin Stabilizing | FXIII | enzyme replacement |
| | factor Def. | | |
| Enzyme | Glutaric acidemia type | lysine oxidase | 3-hydroxyglutaric and |
| | I | | glutaric acid (C5-DC), |
| | | | lysine |
| Enzyme | Gout | Uricase | Uric Acid |
| Enzyme | Gout - hyperuricemia | Uricase | Uric acid (Urate |
| | | | crystals) |
| Enzyme | Hageman Def. | FXII | enzyme replacement |
| Enzyme | Hemolytic anemia due | pyrimidine 5′ | pyrimidines |
| | to pyrimidine 5′ | nucleotidase | |
| | nucleotidase deficiency | | |
| Enzyme | Hemophilia A | Factor VIII | Thrombin (factor II a) |
| | | | or Factor X |
| Enzyme | Hemophilia B | Factor DC | Factor XIa or Factor X |
| Enzyme | Hemophilia C | FXI | enzyme replacement |
| Enzyme | Hepatocellular | Arginine deiminase | Arginine |
| | carcinoma, melanoma | | |
| Enzyme | Homocystinuria | Cystathionine B | homocysteine |
| | | synthase | |
| Enzyme | hyperammonemia/ | Ammonia | Ammonia |
| | ornithinemia/citrullinemia | monooxygenase | |
| | (ornithine transporter | | |
| | defect) | | |
| Enzyme | Isovaleric acidemia | Leucine metabolizing | leucine |
| | | enzyme | |
| Enzyme | Lead poisoning | d-aminolevulinate | lead |
| | | dehydrogenase | |
| Enzyme | Lesch-Nyhan | Uricase | Uric acid |
| | syndrome | | |
| Enzyme | Maple syrup urine | Leucine metabolizing | Leucine |
| | disease | enzyme | |
| Enzyme | Methylmalonic | methylmalonyl-CoA | methylmalonate |
| | acidemia (vitamin b | mutase | |
| | 12 non-responsive) | | |
| Enzyme | Mitochondrial | thymidine | thymidine |
| | neurogastrointestinal | phosphorylase | |
| | encephalomyopathy | | |
| Enzyme | Mitochondrial | Thymidine | Thymidine |
| | neurogastrointestinal | phosphorylase | |
| | encephalomyopathy | | |
| | (MNGIE) | | |
| Enzyme | Owren's disease | FV | enzyme replacement |
| Enzyme | p53-mill solid tumor | Serine dehyrdatase or | serine |
| | | serine hydroxymethyl | |
| | | transferase | |
| Enzyme | Pancreatic | Asparaginase | asparagine |
| | adenocarcinoma | | |
| Enzyme | Phenylketonuria | Phenylalanine | Phenylalanine |
| | | hydroxylase, | |
| | | phenylalanine | |
| | | ammonia lyase | |
| Enzyme | Primary hyperoxaluria | Oxalate oxidase | Oxalate |
| Enzyme | Propionic acidemia | Propionate conversion | Proprionyl coA |
| | | enzyme? | |
| Enzyme | Purine nucleoside | Purine nucleoside | Inosine, dGTP |
| | phosphorylase | phosphorylase | |
| | deficiency | | |
| Enzyme | Stuart-Power Def. | FX | enzyme replacement |
| Enzyme | Thrombotic | ADAMTS 13 | ultra-large von |
| | Thrombocytopenic | | willebrand factor |
| | Purpura | | (ULVWF) |
| Enzyme | Transferase deficient | galactose | Galactose-I -phosphate |
| | galactosemia | dehydrogenase | |
| | (Galactosemia type 1) | | |
| Enzyme | Tyrosinemia type 1 | tyrosine phenol-lyase | tyrosine |
| Enzyme | von Willebrand disease | vWF | enzyme replacement |
| IC clearance | IgA Nephropathy | Complement receptor I | Immune complexes |
| IC clearance | Lupus nephritis | Complement receptor | immune complex |
| | | 1 | |
| IC clearance | Systemic lupus | Complement receptor | immune complex |
| | erythematosus | 1 | |
| Infections | Anthrax (R. anthraris) | an an antibody-like | B. anthraris |
| | infection | binder to B. anthracis | |
| | | surface protein | |
| Infectious | C. botulinum infection | an an antibody-like | C. botilinum |
| | | binder to C. botulinum | |
| | | surface protein | |
| Infectious | C. difficile infection | an antibody-like binder | C. dificile |
| | | to C. difficile surface | |
| | | protein | |
| Infectious | Candida infection | an antibody-like binder | candida |
| | | to candida surface | |
| | | protein | |
| Infectious | E. coli infection | an antibody-like binder | E. coli |
| | | to E. coli surface | |
| | | protein | |
| Infectious | Ebola infection | an antibody-like binder | Ebola |
| | | to Ebola surface | |
| | | protein | |
| Infectious | Hepatitis B (HBV) | an antibody-like binder | HBV |
| | infection | to HBV surface protein | |
| Infectious | Hepatitis C (HCV) | an antibody-like binder | HCV |
| | infection | to HCV surface protein | |
| Infectious | Human | an antibody-like binder | HIV |
| | immunodeficiency | to HIV envelope | |
| | virus (HIV) infection | proteins or CD4 or | |
| | | CCRS or | |
| Infectious | M. tuberculosis | an antibody-like binder | M. tuberculosis |
| | infection | to M. tuberculosis | |
| | | surface protein | |
| Infections | Malaria (P falciparum) | an antibody-like hinder | P falciparum |
| | infection | to P. falriparutm | |
| | | surface protein | |
| Lipid | Hepatic lipase | Hepatic lipase (LIPC) | Lipoprotein, |
| | deficiency, | | intermediate density |
| | hypercholesterolemia | | (IDL) |
| Lipid | Hyperalphalipoproteinemia | Cholesteryl ester | Lipoprotein, high |
| | I | transfer protein(CETP) | density (HDL) |
| Lipid | hypercholesterolemia | an antibody-like binder | LDL |
| | | to low-density | |
| | | lipoprotein (LDL), | |
| | | LDL receptor | |
| Lipid | hypercholesterolemia | an antibody-like binder | HDL |
| | | to high-density | |
| | | lipoprotein (HDL) or | |
| | | HDL receptor | |
| Lipid | lipoprotein lipase | lipoprotein lipase | chilomicrons and very |
| | deficiency | | low density |
| | | | lipoproteins (VLDL) |
| Lipid | Lipoprotein lipase | lipoprotein lipase | Lipoprotein, very low |
| | deficiency, disorders | (LPL) | density (VLDL) |
| | of lipoprotein | | |
| | metabolism | | |
| Lysosomal storage | Aspartylglucosaminuria | N- | glycoproteins |
| | (208400) | Aspartylglucosaminidase | |
| Lysosomal storage | Cerebrotendinous | Sterol 27-hydroxylase | lipids, cholesterol, and |
| | xanthomatosis | | bile acid |
| | (cholestanol lipidosis; | | |
| | 213700) | | |
| Lysosomal storage | Ceroid lipofuscinosis | Palmitoyl-protein | lipopigments |
| | Adult form (CLN4, | thioesterase-1 | |
| | Kufs' disease; 204300) | | |
| Lysosomal storage | Ceroid lipofuscinosis | Paliiiitoyl-protein | lipopigments |
| | Infantile form (CLN1, | thioesterase-1 | |
| | Santavuon-Haltia | | |
| | disease; 256730) | | |
| Lysosomal storage | Ceroid lipofuscinosis | Lysosomal | lipopigments |
| | Juvenile form (CLN3, | transmembrane CLN3 | |
| | Batten disease, Vogt- | protein | |
| | Spielmeyer disease; | | |
| | 204200) | | |
| Lysosomal storage | Ceroid lipofuscinosis | Lysosomal pepstatin- | lipopigments |
| | Late infantile form | insensitive peptidase | |
| | (CLN2, JansIcy- | | |
| | Bielschowslcy disease; | | |
| | 204500) | | |
| Lysosomal storage | Ceroid lipofuscinosis | Transmembrane CLN8 | lipopigments |
| | Progressive epilepsy | protein | |
| | with intellectual | | |
| | disability (600143) | | |
| Lysosomal storage | Ceroid lipofuscinosis | Transmembrane CLN6 | lipopigments |
| | Variant late infantile | protein | |
| | form (CT N6; 601780) | | |
| Lysosomal storage | Ceroid lipofuscinosis | Lysosomal | lipopigments |
| | Variant late infantile | transmembrane CLN5 | |
| | form, Finnish type | protein | |
| | (CLNS; 256731) | | |
| Lysosomal storage | Cholesteryl ester | lysosomal acid lipase | lipids and cholesterol |
| | stomge disease | | |
| | (CESD) | | |
| Lysosomal storage | Congenital disorders of | Phosphomannomutase- | N-glycosylated protein |
| | N-glycosylation CDC | 2 | |
| | la (solely neurologic | | |
| | and neurologic- | | |
| | multivisceral forms; | | |
| | 212065) | | |
| Lysosomal storage | Congenital disorders of | Mannose (Man) | N-glycosylated protein |
| | N-glycosylation CDG | phosphate (P) | |
| | lb (602579) | isomerase | |
| Lysosomal storage | Congenital disorders of | Dolicho-P-GIc: | N-glycosylated protein |
| | N-glycosylation CDG | Man9GIcNAc2-PP- | |
| | Ic (603147) | dolichol | |
| | | glucosyltransferase | |
| Lysosomal storage | Congenital disorders of | Dolicho-P-Man: | N-glycosylated protein |
| | N-glycosylation CDG | Man5GIcNAc2-PP- | |
| | Id (601110) | dolichol | |
| | | mannosyltransferase | |
| Lysosomal storage | Congenital disorders of | Dolichol-P-mannose | N-glycosylated protein |
| | N-glycosylation CDG | synthase | |
| | Ie (608799) | | |
| Lysosomal storage | Congenital disorders of | Protein involved in | N-glycosylated protein |
| | N-glycosylation CDG | mannose-P-dolichol | |
| | If (609180) | utilization | |
| Lysosomal storage | Congenital disorders of | Dolichyl-P-mannose; | N-glycosylated protein |
| | N-glycosylation CDG | Man-7-GIcNAc-2-PP- | |
| | Ig (607143) | dolichyl-\acute{α}-6- | |
| | | mannosyltransferase | |
| Lysosomal storage | Congenital disorders of | Dolichyl-P-glucose: G | N-glycosylated protein |
| | N-glycosylation CDG | lc-1-Man-9-01cNAc- | |
| | lh (608104) | 2-PP-dolichyl-a-3- | |
| | | glucosyltransferase | |
| Lysosomal storage | Congenital disorders of | \acute{α}-1,3- | N-glycosylated protein |
| | N-glycosylation CDG | Mannosyltransferase | |
| | Ii (607906) | | |
| Lysosomal storage | Congenital disorders of | Mannosyl-\acute{α}-1,6- | N-glycosylated protein |
| | N-glycosylation CDG | glycoprotein-β-1, 2-N- | |
| | Ea (212066) | acetylglucosminyltransferase | |
| Lysosomal storage | Congenital disorders of | Glucosidase I | N-glycosylated protein |
| | N-glycosylation CDG | | |
| | fib (606056) | | |
| Lysosomal storage | Congenital disorders of | GDP-fucose | N-glycosylated protein |
| | N-glycosylation CDG | transporter-I | |
| | tic (Rambam-Hasharon | | |
| | syndrome; 266265 | | |
| Lysosomal storage | Congenital disorders of | β-1,4- | N-glycosylated protein |
| | N-glycosylation CDG | Galactosyltransferase | |
| | Ed (607091) | | |
| Lysosomal storage | Congenital disorders of | Oligomeric Golgi | N-glycosylated protein |
| | N-glycosylation CDG | complex-7 | |
| | De (608779) | | |
| Lysosomal storage | Congenital disorders of | UDP-GIcNAc: | N-glycosylated protein |
| | N-glycosylation CDG | dolichyl-P NAcGIc | |
| | Ij (608093) | phosphotransferase | |
| Lysosomal storage | Congenital disorders of | β-1, | N-glycosylated protein |
| | N-glycosylation CDG | 4Mannosyltransferase | |
| | Tk (608540) | | |
| Lysosomal storage | Congenital disorders of | \acute{α}-1,2- | N-glycosylated protein |
| | N-glycosylation CDG | Mannosyltransferase | |
| | II (608776) | | |
| Lysosomal storage | Congenital disorders of | \acute{α}-1,2- | N-glycosylated protein |
| | N-glycosylation, type I | Mannosyltransferase | |
| | (pre-Golgi | | |
| | glycosylation defects) | | |
| Lysosomal storage | Cystinosis | Cystinosin (lysosomal | Cysteine |
| | | cystine transporter) | |
| Lysosomal storage | Fabry's disease | Trihexosylceramide a- | globotriaosylceramide |
| | (301500) | galactosidase | |
| Lysosomal storage | Farber's disease | Ceramidase | lipids |
| | (lipogmnulomatosis; | | |
| | 228000) | | |
| Lysosomal storage | Fucosidosis (230000) | \acute{α}-L-Fucosidase | fucose and complex |
| | | | sugars |
| Lysosomal storage | Galactosialidosis | Protective | lysosomal content |
| | (Goldberg's syndrome, | protein/cathepsin A | |
| | combined | (PPCA) | |
| | neuraminidase and (β- | | |
| | galactosidase | | |
| | deficiency; 256540) | | |
| Lysosomal storage | Gaucher's disease | Glucosylcerainide β- | sphingolipids |
| | | glucosidase | |
| Lysosomal storage | Glutamyl ribose-5- | ADP-ribose protein | glutamyl ribose 5- |
| | phosphate storage | hydrolase | phosphate |
| | disease (305920) | | |
| Lysosomal storage | Glycogen storage | alpha glucosidase | glycogen |
| | disease type 2 | | |
| | (Pompe's disease) | | |
| Lysosomal storage | GM1 gangliosidosis, | Ganglioside β- | acidic lipid material, |
| | generalized | galactosidase | gangliosides |
| Lysosomal storage | GM2 activator protein | GM2 activator protein | gangliosides |
| | deficiency (Tay-Sachs | | |
| | disease AB variant, | | |
| | GM2A; 272750) | | |
| Lysosomal storage | GM2 gangliosidosis | Ganglioside β- | gangliosides |
| | | galactosidase | |
| Lysosomal storage | Infantile sialic acid | Na phosphate | sialic acid |
| | storage disorder | cotransporter, sialin | |
| | (269920) | | |
| Lysosomal storage | Krabbe's disease | Galactosylceramide β- | sphingolipids |
| | (245200) | galactosidase | |
| Lysosomal storage | Lysosomal acid lipase | Lysosomal acid lipase | cholestery esters and |
| | deficiency (278000) | | triglycerides |
| Lysosomal storage | Metachromatic | Arylsul fatase A | sulfatides |
| | leukodystrophy | | |
| | (250100) | | |
| Lysosomal storage | Mucolipidosis ML II | N- | N-linked glycoproteins |
| | (I-cell disease; | Acetylglucosaminyl-1- | |
| | 252500) | phosphotransfeerase | |
| | | catalytic subunit | |
| Lysosomal storage | Mucolipidosis ML III | N-acetylglucosaminyl- | N-linked glycoproteins |
| | (pseudo-Hurler's | 1-phosphotransfeerase | |
| | polydystrophy) | | |
| Lysosomal storage | Mucolipidosis ML III | Catalytic subunit | N-linked glycoproteins |
| | (pseudo-Hurler's | | |
| | polydystrophy) Type | | |
| | 111-A (252600) | | |
| Lysosomal storage | Mucolipidosis ML III | Substrate-recognition | N-linked glycoproteins |
| | (pseudo-Hurler's | subunit | |
| | polydystrophy) Type | | |
| | HI-C (252605) | | |
| Lysosomal storage | Mucopolysaccharidosis | \acute{α}-l-lduronidase | glycosaminoglycans |
| | MPS I HIS (Hurler- | | |
| | Scheie syndrome; | | |
| | 607015) | | |
| Lysosomal storage | Mucopolysaccharidosis | \acute{α}-I-Iduronidase | glycosaminoglycans |
| | MPS I-H (Hurler's | | |
| | syndrome; 607014) | | |
| Lysosomal storage | Mucopolysaccharidosis | Iduronate sulfate | glycosaminoglycans |
| | MPS 11 (Hunter's | sulfatase | |
| | syndrome; 309900) | | |
| Lysosomal storage | Mucopolysaccharidosis | Heparan-S-sulfate | glycosaminoglycans |
| | MPS HI | sulfamidase | |
| | (Sanfilippo's | | |
| | syndrome) Type HI-A | | |
| | (252900) | | |
| Lysosomal storage | Mucopolysaccharidosis | N-acetyl-D- | glycosaminoglycans |
| | MPS HI | glucosaminidase | |
| | (Sanfilippo's | | |
| | syndrome) Type HI-B | | |
| | (252920) | | |
| Lysosomal storage | Mucopolysaccharidosis | Acetyl-CoA- | glycosaminoglycans |
| | MPS HI | glucosaminide N- | |
| | (Sanfilippo's | acetyltranaferase | |
| | syndrome) Type HI-C | | |
| | (252930) | | |
| Lysosomal storage | Mucopolysaccharidosis | N-acetyl- | glycosaminoglycans |
| | MPS HI | glucosaminine-6- | |
| | (Sanfilippo's | sulfate sulfatase | |
| | syndrome) Type HI-D | | |
| | (252940) | | |
| Lysosomal storage | Mucopolysaccharidosis | \acute{α}-I-Iduronidase | glycosaminoglycans |
| | MPS I-S (Seheie's | | |
| | syndrome; 607016) | | |
| Lysosomal storage | Mucopolysaccharidosis | Galactosamine-6- | glycosaminoglycans |
| | MPS IV (Morquio's | sulfate sulfatase | |
| | syndrome) Type IV-A | | |
| | (253000) | | |
| Lysosomal storage | Mucopolysaccharidosis | β-Galactosidase | glycosaminoglycans |
| | MPS IV (Morquio's | | |
| | syndrome) Type IV-B | | |
| | (253010) | | |
| Lysosomal storage | Mucopolysaccharidosis | Hyaltutwidase | glycosaminoglycans |
| | MPS IX | deficiency | |
| | (hyaluronidase | | |
| | deficiency; 601492) | | |
| Lysosomal storage | Mucopolysaccharidosis | N-Acetyl | glycosaminoglycans |
| | MPS VI (Maroteaux- | galactosamine \acute{α}-4- | |
| | Lamy syndrome; | sulfate sulfatase | |
| | 253200) | (aiylsulfatase B) | |
| Lysosomal storage | Mucopolysaccharidosis | β-Glucuronidase | glycosaminoglycans |
| | MPS VII (Sly's | | |
| | syndrome; 253220) | | |
| Lysosomal storage | Mucosulfatidosis | Sulfatase-modifying | sulfatides |
| | (multiple sulfatase | factor-1 | |
| | deficiency; 272200) | | |
| Lysosomal storage | Niemann-Pick disease | Sphingomyelinase | sphingomyelin |
| | type A | | |
| Lysosomal storage | Niemann-Pick disease | Sphingomyelinase | sphingomyelin |
| | type B | | |
| Lysosomal storage | Niemann-Pick disease | NPCI protein | sphingomyelin |
| | Type Cl/Type 13 | | |
| | ((257220) | | |
| Lysosomal storage | Niemann-Pick disease | Epididymal secretory | sphingomyelin |
| | Type C2 (607625) | protein 1 (HE1; NPC2 | |
| | | protein) | |
| Lysosomal storage | Prosaposin deficiency | Prosaposin | sphingolipids |
| | (176801) | | |
| Lysosomal storage | Pycnodysostosis | Cathepsin K | kinins |
| | (265800) | | |
| Lysosomal storage | Sandhofrs disease; | β-Hocosaminidase B | gangliosides |
| | 268800 | | |
| Lysosomal storage | Saposin B deficiency | Saposin B | sphingolipids |
| | (sulfatide activator | | |
| | deficiency) | | |
| Lysosomal storage | Saposin C deficiency | Saposin C | sphingolipids |
| | (Gaucher's activator | | |
| | deficiency) | | |
| Lysosomal storage | Schindler's disease | N-Acetyl- | glycoproteins g |
| | Type I (infantile severe | galactostuninidase | |
| | foray 609241) | | |
| Lysosomal storage | Schindler's disease | N-Acetyl- | lycoproteins |
| | Type II (Kanzaki | galactosaminidase | |
| | disease, adult-onset | | |
| | form; 609242) | | |
| Lysosomal storage | Schindler's disease | N-Acetyl- | glycoproteins |
| | Type HI (intermediate | galactosaminidase | |
| | form; 609241) | | |
| Lysosomal storage | Sialidosis (256550) | Neuriuninidase 1 | mucopolysaccharides |
| | | (sialidase) | and mucolipids |
| Lysosomal storage | Sialuria Finnish type | Na phosphate | sialic acid |
| | (Salta disease; 604369) | cotransporter, sialin | |
| Lysosomal storage | Sialuria French type | UDP-N- | sialic acid |
| | (269921) | acetylglucosamine-2- | |
| | | epimerase/N- | |
| | | acetylmannosamine | |
| | | kinase, sialin | |
| Lysosomal storage | Sphingolipidosis Type | Ganglioside β- | sphingolipids |
| | I (230500) | galactosidase | |
| Lysosomal storage | Sphingolipidosis Type | Ganglioside β- | sphingolipids |
| | II (juvenile type; | galactosidase | |
| | 230600) | | |
| Lysosomal storage | Sphingolipidosis Type | Ganglioside β- | sphingolipids |
| | III (adult type; | galactosidase | |
| | 230650) | | |
| Lysosomal storage | Tay-Sachs disease; | β-Hexosaminidase A | gangliosides |
| | 272800 | | |
| Lysosomal storage | Winchester syndrome | Metal loproteinase- 2 | mucopolysaccharides |
| | (277950) | | |
| Lysosomal storage | Wolman's disease | lysosomal acid lipase | lipids and cholesterol |
| Lysosomal storage | \acute{α}-Mannosidosis | \acute{α}-D-Mannosidase | carbohydrates and |
| | (248500). type I | | glycoproteins |
| | (severe) or II (mild) | | |
| Lysosomal storage | β-Mannosidosis | β-D-Mannosidase | carbohydrates and |
| | (248510) | | glycoproteins |
| Toxic Molecule | alpha hemolysin | an antibody-like binder | alpha hemolysin |
| | poisoning | to alpha hemolysin | |
| Toxic Molecule | antrax toxin poisoning | an antibody-like binder | anthrax toxin |
| | | to anthrax toxin | |
| Toxic Molecule | bacterial toxin-induced | an antibody-like binder | bacterial toxin |
| | shock | to bacterial toxin | |
| Toxic Molecule | botulinum toxin | an antibody-like binder | botulinum toxin |
| | poisoning | to botulinum toxin | |
| Toxic Molecule | Hemochromatosis | iron chelator | molecular iron |
| | (iron poisoning) | | |
| Toxic Molecule | Methanol poisoning | Methanol | Methanol |
| | | dehdrogenase | |
| Toxic Molecule | Nerve gas poisoning | Butyryl cholinesterase | Sarin |
| Toxic Molecule | Prion disease caused | an antibody-like binder | Prion protein PRP |
| | by PRP | to prion protein PRP | |
| Toxic Molecule | Prion disease caused | an antibody-like binder | Prion protein PRPc |
| | by PRPc | to prion protein PRPc | |
| Toxic Molecule | Prion disease caused | an antibody-like binder | Prion protein PRPsc |
| | by | to prion protein PRPsc | |
| Toxic Molecule | PRPsc Prion disease | an antibody-like binder | Prion protein PRPres |
| | cussed by PRPrcs | to prion protein | |
| | | PRPres | |
| Toxic Molecule | Sepsis or cytokine | an antibody-like binder | cytokines |
| | storm | to cytokines or Duffy | |
| | | antigen receptor of | |
| | | chemokines (DARC) | |
| Toxic Molecule | spider venom | an antibody-like binder | spider venom |
| | poisoning | to spider venom | |
| Toxic Molecule | Wilson disease | copper chelator | molecular copper |

TABLE 14
Cytokines and Receptors

| Name | Cytokine Receptor(s)(Da) and Form |
|---|---|
| Interleukins | |
| IL-1-like | |
| IL-1α | CD121a, CDw121b |
| IL-1β | CD121a, CDw121b |
| IL-1RA | CD121a |
| IL-18 | IL-18Rα, β |
| Common g chain (CD132) | |
| IL-2 | CD25, 122, 132 |
| IL-4 | CD124, 213a13, 132 |
| I1-7 | CD127, 132 |
| IL-9 | IL-9R, CD132 |
| IL-13 | CD213a1, 213a2, |
| IL-15 | IL-15Ra, CD122, 132 |
| Common b chain (CD131) | |
| IL-3 | CD123, CDw131 |
| IL-5 | CDw125, 131 |
| Also related | |
| GM-CSF | CD116, CDw131 |
| IL-6-like | |
| IL-6 | CD126, 130 |
| IL-11 | IL-11Ra, CD130 |
| Also related | |
| G-CSF | CD114 |
| IL-12 | CD212 |
| LIF | LIFR, CD130 |
| OSM | OSMR, CD130 |
| IL-10-like | |
| IL-10 | CDw210 |
| IL-20 | IL-20Rα, β |
| Others | |
| IL-14 | IL-14R |
| IL-16 | CD4 |
| IL-17 | CDw217 |
| Interferons | |
| IFN-α | CD118 |
| IFN-β | CD118 |
| IFN-γ | CDw119 |
| TNF | |
| CD154 | CD40 |
| LT-β | LT-βR |
| TNF-α | CD120a, b |
| TNF-β (LT-α) | CD120a, b |
| 4-1BBL | CD137 (4-1BB) |
| APRIL | BCMA, TACI |
| CD70 | CD27 |
| CD153 | CD30 |
| CD178 | CD95 (Fas) |
| GITRL | GITR |
| LIGHT | LTbR, HVEM |
| OX40L | OX40 |
| TALL-1 | BCMA, TACI |
| TRAIL | TRAILR1-4 |
| TWEAK | Apo3 |
| TRANCE | RANK, OPG |
| TGF-β | |
| TGF-β1 | TGF-βR1 |
| TGF-β2 | TGF-βR2 |
| TGF-β3 | TGF-βR3 |
| Miscellaneous hematopoietins | |
| Epo | EpoR |
| Tpo | TpoR |
| Flt-3L | Flt-3 |
| SCF | CD117 |
| M-CSF | CD115 |
| MSP | CDw136 |

TABLE 15
T cell activation

| Activating Ligand | Target Receptor on T cell |
|---|---|
| B7-H2 (e.g., Accession Number | ICOS, CD28 (e.g., Accession |
| NP_056074.1) | Number NP_006130.1) |
| B7-1 (e.g., Accession Number | CD28 (e.g., Accession Number |
| NP_005182.1) | NP_006130.1) |
| B7-2 (e.g., Accession Number | CD28 (e.g., Accession Number |
| AAA86473) | NP_006130.1) |
| CD70 (e.g., Accession Number | CD27 (e.g., Accession Number |
| NP_001243.1) | NP_001233.1) |
| LIGHT (e.g., Accession Number | HVEM (e.g., Accession Number |
| NP_003798.2) | AAQ89238.1) |
| HVEM (e.g., Accession Number | LIGHT (e.g., Accession Number |
| AAQ89238.1) | NP_003798.2) |
| CD40L (e.g., Accession Number | CD40 (e.g., Accession Number |
| BAA06599.1) | NP_001241.1) |
| 4-1BBL (e.g., Accession Number | 4-IF3B (e.g., Accession Number |
| NP_003802.1) | NP_001552.2) |
| OX40L (e.g., Accession Number | OX40 (e.g., Accession Number |
| NP_003317.1) | NP_003318.1) |
| TLIA (e.g., Accession Number | DR3 (e.g., Accession Number |
| NP_005109.2) | NP_683866.1) |
| GITRL (e.g., Accession Number | GITR (e.g., Accession Number |
| NP_005083.2) | NP_004186.1) |
| CD30L (e.g., Accession Number | CD30 (e.g., Accession Number |
| NP_001235.1), | NP_001234.3) |
| TIM4 (e.g., Accession Number | TIM1 (e.g., Accession Number |
| NP_612388.2) | NP_036338.2) |
| SLAM (e.g., Accession Number | SLAM (e.g., Accession Number |
| AAK77968.1) | AAK77968.1) |
| CD48 (e.g., Accession Number | CD2 (e.g., Accession Number |
| CA033293.1) | NP_001315538.1) |
| CD58 (e.g., Accession Number | CD2 (e.g., Accession Number |
| CAG33220.1) | NP_001315538.1) |
| CDI55 (e.g., Accession Number | CD226 (e.g., Accession Number |
| NP_001129240.1) | NP_006557.2) |
| CD112 (e.g., Accession Number | CD226 (e.g., Accession Number |
| NP_001036189.1) | NP_006557.2) |
| CD137L (e.g., Accession Number | CD137 (e.g., Accession Number |
| NP_003802.1) | NP_001552.2) |

TABLE 16
T cell inhibition

| Inhibitory Ligand | Target Receptor on T cell |
|---|---|
| B7-1 | CTLA4, B7H1 |
| B7-2 | CTLA4 |
| B7DC | PD1 |
| B7H1 | PD1, B7-1 |
| HVEM | CD160, BTLA |
| COLLAGEN | LAIR1 |
| GALECTIN9 | TIM3 |
| CD48, TIM4 | TIM4R |
| CD48 | 2B4 |
| CD155, CD112, | TIGIT |
| CD113 | |
| PDL1 | PD1 |

In an aspect of the present disclosure, the pharmaceutical agent is an anti-cancer therapy. In another aspect, the pharmaceutical agent is a therapeutic anti-cancer antibody as provided by US Patent Publication No. 2017/0020926, published Jan. 26, 2019, and

Table 17, below. In another aspect, the pharmaceutical agent is an agent that binds to an immune checkpoint molecule or costimulatory molecule as provided by tables 4 and 5 of US Patent Publication No. 2017/0020926, published Jan. 26, 2019, and Table 18 and Table 19. In a further aspect, the pharmaceutical agent targets any of the diseases and targets provided in US Patent Publication No. 2018/0135012, published May 17, 2018 and U.S. Pat. No. 9,664,180.

In an aspect of the present disclosure, methods of loading a pharmaceutical agent to a red blood cell include electroporation, endocytosis, osmotic based like hypnotic dilution, dialysis, osmotic lysis, lipid fusion, and chemical perturbation. In another aspect, a method for incorporating a pharmaceutical agent is selected from the group consisting of electroporation, osmotic shock, hypotonic and hypertonic cycling, cell surface labeling with heterobifunctional crosslinking, and cell surface labeling with click chemistry. The methods of loading the pharmaceutical agent are understood by those skilled in the art. See Dischmukhe A and Shetty S: Resealed erythrocytes: a novel and promising drug carrier. Int J Pharm Sci Res 8(8):3242-51 (2017).

TABLE 17
Therapeutic anti-cancer antibodies

| Antibody | Target | Exemplary indications |
|---|---|---|
| rituximab | CD20 | Lymphoma e.g., NHL, leukemia |
| alemtuzumab | CD52 | Leukemia, e.g., B cell leukemia |
| Trasmzumab, e.g., | HER2/neu | Solid rumors, e.g., breast cancer, e.g., |
| ado-Lrastuzumab, | | HER2- |
| e.g.; ado- | | positive breast cancer, e.g., breast cancer |
| trastuzumab | | with HER2 overexpression |
| emtansine | | |
| nimotuzumab | EGFR | Solid tumors, e.g., squamous cell carcinoma |
| | | or glioma |
| cetuximab | EGFR | Solid tumors, e.g., squamous cell carcinoma |
| | | or colorectal carcinoma |
| bevacizwnab | VEGF | Solid tumors, e.g., colon cancer, lung |
| | | cancer, renal cancer, ovarian cancer, |
| | | glioma, breast cancer |
| bavituximab | Phosphatidylserine | Solid tumors, e.g., lung cancer e.g. non- |
| | | small cell lung cancer, pancreatic cancer, |
| | | hepatocellular carcinoma, melanoma, rectal |
| | | cancer, prostate cancer |
| gemtuzumab, e.g., | CD33 | Leukemia, e.g., acute myelogenous |
| gemtuzumab | | leukemia |
| ozogamicin | | |
| ibritumomab, e.g., | CD20 | Lymphoma, e.g., NHL, e.g., B cell non- |
| ibritumomab | | Hodgkin's lymphoma |
| tiuxetan | | Lymphoma: e.g., NHL |
| Tositumomab, e.g., | | |
| tositumomab-1-131 | | |
| panitumumab | EGFR | Solid tumors, e.g., colorectal cancer |
| ofatumumab | CD20 | Leukemia, e.g., CLL, lymphoma e.g., NHL, |
| | | e.g., DLBCL |
| ipilimwnab | CTLA-4 | Solid tumors, e.g., melanoma, bladder |
| | | cancer, prostate cancer, and lung cancer e.g. |
| | | NSCLC and SCLC |
| brentuximab: e.g., | CD30 | Lymphoma, e.g., Hodgkin lymphoma and |
| brentuximab vedotin | | sALCL |
| pertuzwnab | Her2 | Solid tumors, e.g., breast cancer, e.g., |
| | | HER2- positive breast cancer, e.g., breast |
| | | cancer with HER2 overexpression |
| obinutuzumab | CD20 | Leukemia, e.g., CLL, and lymphoma, e.g., |
| | | follicular lymphoma |
| durvalwnab | PD-L1 | Solid tumors, e.g., bladder cancer and lung |
| | | cancer e.g., NSCLC |
| nivolumab | PD-1 | Solid tumors, e.g., renal cell carcinoma, |
| | | melanoma or lung cancer e.g., NSCLC |
| pembrolizumab | PD-1 | Solid tumors, e.g., HNSCC, melanoma, or |
| | | lung cancer e.g., NSCLC |
| olaratumab | PDGF-R a | Solid tumors, e.g., soft tissue sarcoma |
| atezolizwnab | PD-L1 | Solid tumors, e.g., lung cancer, e.g., |
| | | NSCLC |
| elotuzumab | SLAMF7 | Multiple myeloma |
| | (CD319) | |
| necitwuwuab | EGFR | Solid tumors, e.g., lung cancer, e.g., |
| | | NSCLC |
| daratumumab | CD38 | Multiple myeloma |
| ramucirumab | VEGFR2 (KDR) | Solid tumors, e.g., colorectal cancer |
| dinutuximab | GD2 | Solid tumors, e.g., neuroblastoma |
| blinatumomab | CD19, CD3 | Leukemia, e.g., ALL |
| siltuximab | IL-6 | solid tumors, e.g., renal cell cancer, prostate |
| | | cancer |
| denosumab | RANK ligand | Solid tumors, e.g., giant cell tumor of bone |
| catumaxomab | EpCAM, CD3 | Solid tumors, e.g., head and neck cancer |
| enoblituzumab | B7H3 | Solid tumors, e.g., NSCLC, SCCHN, |
| | | Melanoma, Urothelial Cancer |
| tremdimumab | CTLA4 | Solid tumors, e.g., Melanoma, |
| | | mesothelioma |
| lirilumab | KIR2D subgroup | Leukemia, e.g., AML, CLL; solid tumors |
| pidilizwnab | PD1 | Lymphoma, e.g., DLBCL; multiple |
| | | myeloma |
| avelwnab | PDL1 | Solid tumors, e.g., Nasopharyngeal Cancer, |
| | | Endometrial Cancer, Merkel Cell |
| | | Carcinoma, Ovarian Cancer, Peritoneal |
| | | Cancer, Fallopian Tube Cancer; Leukemia |
| | | e.g., AML |

TABLE 18
Immune checkpoint molecules, and agents that bind thereto.

| | Antibody or other binding agent (e.g., |
|---|---|
| Immune checkpoint molecule | immune checkpoint molecule inhibitors) |
| A2aR | Anti-Adenosine Receptor A2a Antibody, clone 7F6-G5-A2 |
| | (EMD Millipore) |
| 2B4 (also called CD244 or | CD48 (e.g., Accession Number CAG33293.1) |
| SLAME4) | |
| B-7 family ligand, e.g., B7-H3 | Enoblituzumab |
| B7-H4 | h1D11 TDC (see Leong et al., doi: 10.1021/mp5007745) |
| BTLA | HVEM (e.g., Accession Number AA.Q89238.1) |
| CGEN-15049 | Anti- CGEN-15049 antibody |
| CD160 | HVEM (e.g., Accession Number AAQ89238.1) |
| CEACAM (e.g, CEACAM- 1, | Amexin, e.g., Annexin A2 (e.g., Accession Number |
| CEACAM-3 and/or | AAH52558.1) |
| CEACAM-5) | |
| CHK1 | 2G1D5 mAb (Cell Signalling Technology) |
| CHK2 | 1C12 mAb #34.40 (Cell Signalling Technology) |
| CTLA4 | B7-1 (CD80; e.g., Accession Number N13_005182.1); |
| | B7-2 (CD86; e.g., Accession Number NP 787058.4); |
| | ipilimumab, tremelimumab |
| GAL9 | Anti-human Galectin-9 Antibody 9M1-3 (BioLegend) |
| GR1 | RB6-8C5 antibody (eBioscience) |
| HVEM | BTLA (e.g., Accession Number NP,_861445.3), CD160 |
| | (e.g., Accession Number NP 008984.1) |
| KIR | Lirilumab |
| LAG3 | BMS-986016 |
| LAIR1 | COLLAGEN; Anti-Human CD305 (eBioscience) |
| LY6G | 1A8 antibody (eBioscience) |
| MUC1 | Anti-MUC1 antibody EPR1023 (Abeam) |
| PD1 | B7DC; PD-L1; nivolumab; pembrolizumab. pidilizumab |
| PD-L1 (also called B7H1) | B7-1 (CD80; e.g., Accession Number NP 005182.1); PD1 |
| | (e.g., Accession Number NP 005009.2); durvalumab; |
| | atezolizumab; avelumab; BMS 936559 |
| PDL2 | AMP 224 |
| TGF beta | Antibody 1D11 (see Ueda R et al., doi: 10.1158/1078- |
| | 0432.CCR-09-1067) |
| TIGIT | CD155 (e.g., Accession Number NP_001129240.1); |
| | CD112 (e.g., Accession Number NP 001036189.1); |
| | CD113 (e.g., Accession Number NP 001230215.1); |
| TIM3 | GALECTIN9; |
| TIM4 | TIM4R; CD48 (e.g., Accession Number NP_001769.2) |
| TIM4R | TIM4 (e.g., Accession Number NP 612388.2) |
| VISTA | Human VISTA Antibody Clone # 730804 (R&D Systems) |

TABLE 19
Costimulatory molecules, and agents that bind thereto.

| Costimulatory molecule | Antibody or other binding agent |
|---|---|
| 4-1BB (CD137; e.g., | 4-1BBL (e.g., Accession Number NP 003802.1), |
| Accession NP 001552.2) | CD137L (e.g., Accession Number NP 003802.1) |
| CD2 (e.g., Accession Number | CD48 (e.g., Accession Number NP_001769.2) |
| NP_001315538.1) | |
| CD27 (e.g., Accession Number | CD70 (e.g., Accession Number NP 001243.1) |
| NP_001233.1) | |
| CD28 (e.g., Accession Number | B7-H2 (e.g., Accession Number NP 056074.1), |
| NP 006130.1) | B7-1 (CD80; e.g., Accession Number NP 005182.1) |
| CD30 (e.g., Accession Number | CD3OL (e.g., Accession Number NP 001235.1), |
| NP 001234.3) | brentaximab |
| CD40 (e.g., Accession Number | CD4OL (e.g., Accession Number BAA06599.1) |
| NP_001241.1) | |
| CD226 (e.g., Accession Number | CD155 (e.g., Accession Number NP_001129240.1), |
| NP_006557.2) | CD112 (e.g., Accession Number NP 001036189.1) |
| CDS | |
| DR3 (e.g., Accession Number | TL1A (e.g., Accession Number NP 005109.2) |
| NP 683866.1) | |
| GITR (e.g., Accession Number | GITRL (e.g., Accession Number NP 05083.2) |
| NP 004186.1) | |
| HVEM (LIGHTR) (e.g., | LIGHT (e.g., Accession Number NP 003798.2) |
| Accession Number | |
| AAQ89238.1) | |
| ICAM-1 (e.g., Accession | Anti-ICAM1 antibody [MEM-111] (Abeam) |
| Number NP 000192.2) | |
| LFA-1 (CD11a/CD18) (e.g., | odulimomab |
| Accession Number | |
| NP_002200.2) | |
| LIGHT (e.g., Accession | HVEM (e.g., Accession Number AAQ89238.1) |
| Number NP 003798.2) | |
| ICOS (CD278) (e.g., | B7-H2 (e.g., Accession Number NP 056074.1) |
| Accession | |
| Number NP_036224.1) | |
| OX40 (e.g., Accession | OX4OL (e.g., Accession Number NP 003317.1) |
| Number NP 003318.1) | |
| TIM1 (e.g., Accession Number | TIM4 (e.g., Accession Number NP 612388.2) |
| NP 036338.2) | |

In an aspect of the present disclosure, red blood cells are treated with a chemical agent to prepare a red blood cell comprising a hemoglobin derivative. In an aspect, the chemical agent is carbon monoxide and the hemoglobin derivative is carboxy-hemoglobin. In another aspect, the chemical agent is cyanide and the hemoglobin derivative is cyano-methemoglobin. In an aspect, hemoglobin is oxidized first by an oxidizing agent, such as methylene blue or potassium ferricyanide then reacted with NaCN solution or HCN gas to form cyano-methemoglobin. In a further aspect, the agent is azide (N₃) and the hemoglobin derivative is azido-methemoglobin formed by reacting with aqueous solution of sodium azide. In another aspect, the agent is an agent capable of binding and stabilizing the hemoglobin molecule.

In an aspect of the present disclosure, increasing the shelf-life of a red blood cell composition comprising carboxyhemoglobin includes increasing the maximum length of refrigerated or room temperature storage time of the red blood cell composition while retaining the ability of the red blood cell composition to circulate in a patient in need thereof. In another aspect, increasing the shelf-life of a red blood cell composition comprising carboxyhemoglobin includes increasing the maximum length of refrigerated or ambient temperature storage time while minimizing formation of Heinz bodies. In yet another aspect, increasing the shelf-life of a red blood cell composition comprising carboxyhemoglobin comprises decreasing the number of Heinz bodies formed by at least 5, 10, 15, 20, 25, 30, 35, 40, 45, 50, 55, 60, 65, 70% or more relative to RBCs comprising oxyhemoglobin and stored under similar conditions. In another aspect, the Heinz bodies are decreased by between 5 and 10, 10 and 20, 20 and 30, 30 and 40, 40 and 50, 50 and 60, or 60 and 70% in a red blood cell composition comprising carboxyhemoglobin relative to RBCs comprising oxyhemoglobin and stored under similar conditions. In another aspect, increasing the shelf-life of a red blood cell composition comprising carboxyhemoglobin includes reducing the number of lysed cells relative to RBCs comprising oxyhemoglobin and stored under similar conditions. In an aspect, the number of lysed cells is decreased by at least 5, 10, 15, 20, 25, 30, 35, 40, 45, 50, 55, 60, 65, 70% or more in a red blood cell composition comprising carboxyhemoglobin relative to RBCs comprising oxyhemoglobin and stored under similar conditions.

In an aspect of the present disclosure, increasing the shelf-life of a red blood cell composition comprising cyano-methemoglobin includes increasing the maximum length of refrigerated or room temperature storage time of the red blood cell composition while retaining the ability of the red blood cell composition to circulate in a patient in need thereof. In another aspect, increasing the shelf-life of a red blood cell composition comprising cyano-methemoglobin includes increasing the maximum length of refrigerated or ambient temperature storage time without forming Heinz bodies. In yet another aspect, increasing the shelf-life of a red blood cell composition comprising cyano-methemoglobin comprises decreasing the number of Heinz bodies formed by at least 5, 10, 15, 20, 25, 30, 35, 40, 45, 50, 55, 60, 65, 70% or more relative to RBCs comprising oxyhemoglobin and stored under similar conditions. In another aspect, the Heinz bodies are decreased by between 5 and 10, 10 and 20, 20 and 30, 30 and 40, 40 and 50, 50 and 60, or 60 and 70% in a red blood cell composition comprising cyano-methemoglobin relative to RBCs comprising oxyhemoglobin and stored under similar conditions. In another aspect, increasing the shelf-life of a red blood cell composition comprising cyano-methemoglobin includes reducing the number of lysed cells relative to RBCs comprising oxyhemoglobin and stored under similar conditions. In an aspect, the number of lysed cells is decreased by at least 5, 10, 15, 20, 25, 30, 35, 40, 45, 50, 55, 60, 65, 70% or more in a red blood cell composition comprising cyano-methemoglobin hemoglobin relative to RBCs comprising oxyhemoglobin and stored under similar conditions.

In an aspect of the present disclosure, increasing the shelf-life of a red blood cell composition comprising azido-hemoglobin includes increasing the maximum length of refrigerated or room temperature storage time of the red blood cell composition while retaining the ability of the red blood cell composition to circulate in a patient in need thereof. In another aspect, increasing the shelf-life of a red blood cell composition comprising azido-hemoglobin includes increasing the maximum length of refrigerated or ambient temperature storage time without forming Heinz bodies. In yet another aspect, increasing the shelf-life of a red blood cell composition comprising azido-hemoglobin comprises decreasing the number of Heinz bodies formed by at least 5, 10, 15, 20, 25, 30, 35, 40, 45, 50, 55, 60, 65, 70% or more relative to RBCs comprising oxyhemoglobin and stored under similar conditions. In another aspect, the Heinz bodies are decreased by between 5 and 10, 10 and 20, 20 and 30, 30 and 40, 40 and 50, 50 and 60, or 60 and 70% in a red blood cell composition comprising azido-hemoglobin relative to RBCs comprising oxyhemoglobin and stored under similar conditions. In another aspect, increasing the shelf-life of a red blood cell composition comprising azido-hemoglobin includes reducing the number of lysed cells relative to RBCs comprising oxyhemoglobin and stored under similar conditions. In an aspect, the number of lysed cells is decreased by at least 5, 10, 15, 20, 25, 30, 35, 40, 45, 50, 55, 60, 65, 70% or more in a red blood cell composition comprising azido-methemoglobin relative to RBCs comprising oxyhemoglobin and stored under similar conditions.

In an aspect of the present disclosure, shelf-life is measured by quantifying the efficacy of the red blood cell comprising a pharmaceutical agent and carboxyhemoglobin. In another aspect of the present disclosure, shelf-life is measured by quantifying the efficacy of the red blood cell comprising a pharmaceutical agent and cyano-methemoglobin. In another aspect of the present disclosure, shelf-life is measured by quantifying the efficacy of the red blood cell comprising a pharmaceutical agent and azido-hemoglobin. In another aspect, shelf-life is measured by labeling a red blood cell comprising a pharmaceutical agent with biotin, ⁵¹Cr, or ^{99m}Tc and quantifying the length of time in circulation.

In an aspect of the present disclosure, the shelf-life of RBCs comprising carboxyhemoglobin is increased by one or more weeks relative to RBCs comprising oxyhemoglobin and stored under similar conditions. In another aspect, the shelf-life of RBCs comprising carboxyhemoglobin is increased by two or more weeks relative to RBCs comprising oxyhemoglobin and stored under similar conditions. In another aspect, the shelf-life of RBCs comprising carboxyhemoglobin is increased by three or more weeks relative to RBCs comprising oxyhemoglobin and stored under similar conditions. In another aspect, the shelf-life of RBCs comprising carboxyhemoglobin is increased by four or more weeks relative to RBCs comprising oxyhemoglobin and stored under similar conditions. In another aspect, the shelf-life of RBCs comprising carboxyhemoglobin is increased by five or more weeks relative to RBCs comprising oxyhemoglobin and stored under similar conditions. In another aspect, the shelf-life of RBCs comprising carboxyhemoglobin is increased by two or more days relative to RBCs comprising oxyhemoglobin and stored under similar conditions. In another aspect, the shelf-life of RBCs comprising carboxyhemoglobin is increased by four or more days relative to RBCs comprising oxyhemoglobin and stored under similar conditions. In another aspect, the shelf-life of RBCs comprising carboxyhemoglobin is increased by between 3 and 10 days, 5 and 10 days, 7 and 14 days, or 5 and 30 days relative to RBCs comprising oxyhemoglobin and stored under similar conditions. In yet another aspect, the shelf-life of RBCs comprising carboxyhemoglobin is increased by between 1 and 2 weeks, 1 and 3 weeks, 1 and 4 weeks, 1 and 5 weeks, or 2 and 10 weeks relative to RBCs comprising oxyhemoglobin and stored under similar conditions. In another aspect, the shelf-life of RBCs comprising carboxyhemoglobin is increased by between 5 and 10%, 10 and 20%, 20 and 40%, 40 and 60%, 60 and 80%, or 80 and 100% relative to RBCs comprising oxyhemoglobin and stored under similar conditions. In another aspect, the shelf-life is increased by at least 10% relative to RBCs comprising oxyhemoglobin and stored under similar conditions. In another aspect, the shelf-life is increased by at least 20% relative to RBCs comprising oxyhemoglobin and stored under similar conditions. In another aspect, the shelf-life is increased by at least 30% relative to RBCs comprising oxyhemoglobin and stored under similar conditions. In another aspect, the shelf-life is increased by at least 40% relative to RBCs comprising oxyhemoglobin and stored under similar conditions. In another aspect, the shelf-life is increased by at least 50% relative to RBCs comprising oxyhemoglobin and stored under similar conditions.

In another aspect, the shelf-life of RBCs comprising cyano-methemoglobin is increased by one or more weeks relative to RBCs comprising oxyhemoglobin and stored under similar conditions. In another aspect, the shelf-life of RBCs comprising cyano-methemoglobin is increased by two or more weeks relative to RBCs comprising oxyhemoglobin and stored under similar conditions. In another aspect, the shelf-life of RBCs comprising cyano-methemoglobin is increased by three or more weeks relative to RBCs comprising oxyhemoglobin and stored under similar conditions. In another aspect, the shelf-life of RBCs comprising cyano-methemoglobin is increased by four or more weeks relative to RBCs comprising oxyhemoglobin and stored under similar conditions. In another aspect, the shelf-life of RBCs comprising cyano-methemoglobin is increased by five or more weeks relative to RBCs comprising oxyhemoglobin and stored under similar conditions. In another aspect, the shelf-life of RBCs comprising cyano-methemoglobin is increased by two or more days relative to RBCs comprising oxyhemoglobin and stored under similar conditions. In another aspect, the shelf-life of RBCs comprising cyano-methemoglobin is increased by four or more days relative to RBCs comprising oxyhemoglobin and stored under similar conditions. In another aspect, the shelf-life is increased by between 3 and 10 days, 5 and 10 days, 7 and 14 days, or 5 and 30 days relative to RBCs comprising oxyhemoglobin and stored under similar conditions. In yet another aspect, the shelf-life of RBCs comprising cyano-methemoglobin is increased by between 1 and 2 weeks, 1 and 3 weeks, 1 and 4 weeks, 1 and 5 weeks, or 2 and 10 weeks relative to RBCs comprising oxyhemoglobin and stored under similar conditions. In another aspect, the shelf-life of RBCs comprising cyano-methemoglobin is increased by between 5 and 10%, 10 and 20%, 20 and 40%, 40 and 60%, 60 and 80%, or 80 and 100% relative to RBCs comprising oxyhemoglobin and stored under similar conditions. In another aspect, the shelf-life is increased by at least 10% relative to RBCs comprising oxyhemoglobin and stored under similar conditions. In another aspect, the shelf-life is increased by at least 20% relative to RBCs comprising oxyhemoglobin and stored under similar conditions. In another aspect, the shelf-life is increased by at least 30% relative to RBCs comprising oxyhemoglobin and stored under similar conditions. In another aspect, the shelf-life is increased by at least 40% relative to RBCs comprising oxyhemoglobin and stored under similar conditions. In another aspect, the shelf-life is increased by at least 50% relative to RBCs comprising oxyhemoglobin and stored under similar conditions.

In an aspect of the present disclosure, the shelf-life of RBCs comprising azido-methemoglobin is increased by one or more weeks relative to RBCs comprising oxyhemoglobin and stored under similar conditions. In another aspect, the shelf-life of RBCs comprising azido-methemoglobin is increased by two or more weeks relative to RBCs comprising oxyhemoglobin and stored under similar conditions. In another aspect, the shelf-life of RBCs comprising azido-methemoglobin is increased by three or more weeks relative to RBCs comprising oxyhemoglobin and stored under similar conditions. In another aspect, the shelf-life of RBCs comprising azido-methemoglobin is increased by four or more weeks relative to RBCs comprising oxyhemoglobin and stored under similar conditions. In another aspect, the shelf-life is increased by five or more weeks relative to RBCs comprising oxyhemoglobin and stored under similar conditions. In another aspect, the shelf-life is increased by two or more days relative to RBCs comprising oxyhemoglobin and stored under similar conditions. In another aspect, the shelf-life is increased by four or more days relative to RBCs comprising oxyhemoglobin and stored under similar conditions. In another aspect, the shelf-life is increased by between 3 and 10 days, 5 and 10 days, 7 and 14 days, or 5 and 30 days relative to RBCs comprising oxyhemoglobin and stored under similar conditions. In yet another aspect, the shelf-life is increased by between 1 and 2 weeks, 1 and 3 weeks, 1 and 4 weeks, 1 and 5 weeks, or 2 and 10 weeks relative to RBCs comprising oxyhemoglobin and stored under similar conditions. In another aspect, the shelf-life of RBCs comprising azido-methemoglobin is increased by between 5 and 10%, 10 and 20%, 20 and 40%, 40 and 60%, 60 and 80%, or 80 and 100% relative to RBCs comprising oxyhemoglobin and stored under similar conditions. In another aspect, the shelf-life is increased by at least 10% relative to RBCs comprising oxyhemoglobin and stored under similar conditions. In another aspect, the shelf-life is increased by at least 20% relative to RBCs comprising oxyhemoglobin and stored under similar conditions. In another aspect, the shelf-life is increased by at least 30% relative to RBCs comprising oxyhemoglobin and stored under similar conditions. In another aspect, the shelf-life is increased by at least 40% relative to RBCs comprising oxyhemoglobin and stored under similar conditions. In another aspect, the shelf-life is increased by at least 50% relative to RBCs comprising oxyhemoglobin and stored under similar conditions.

The present disclosure provides for a storage atmosphere for storing a red blood cell composition for drug delivery. In an aspect, the storage atmosphere comprises minimal partial pressure of oxygen. In an aspect, the storage atmosphere comprises less than 20 mmHg, 15 mmHg, or 10 mmHg. In another aspect, the storage atmosphere comprises minimal partial pressure of oxygen relative to the partial pressure of nitrogen, argon, helium, or carbon monoxide. In an aspect, a red blood cell composition for drug delivery comprises carboxyhemoglobin, cyano-methemoglobin, or azido-methemoglobin.

In an aspect, the storage atmosphere is contained in a vial, a container, a syringe, or a bag. In another aspect, storage is under ambient pressure. In another aspect, the storage atmosphere comprises ambient air, carbon monoxide, N₂, or a combination thereof. In a further aspect, the storage atmosphere comprises less than 20, 15, 10, 5, or 3% saturated oxygen. In another aspect, the storage atmosphere comprises between 3 and 5, 5 and 10, 10 and 15, or 15 and 20% saturated oxygen.

The present disclosure provides for treating red blood cells comprising a pharmaceutical agent with gas exchange. In an aspect, the present disclosure provides for treating red blood cells comprising a pharmaceutical agent and oxyhemoglobin with gas exchange. In an aspect, gas exchange comprises rapid gas exchange. In another aspect, gas exchange comprises overnight gas exchange. In yet another aspect, gas exchange comprises membrane gas exchange. In a further aspect, gas exchange comprises microbubble gas exchange. In another aspect, gas exchange is with carbon monoxide, HCN gas, or NAN3 solution.

In an aspect of the present disclosure, the storage temperature is between 0.1 and 6° C., −4 and 6° C., 6 and 24° C., 24 and 38° C. In another aspect, the storage temperature is greater than −4° C., 4° C., 10° C., 15° C., 20° C., 25° C., 30° C., 35° C., or 40° C. In another aspect the storage temperature is about 37° C. In yet another aspect, the storage temperature is about 27° C. In some aspects, the compositions further comprise a cryoprotectant. In yet another aspect, the storage temperature is about −80° C.

In an aspect of the present disclosure, a red blood cell composition comprises an additive solution. In another aspect, the additive solution comprises one or more of glucose, phosphate, citrate, bicarbonate, or sodium chloride (NaCl). In another aspect, a red blood cell composition comprising an additive solution has a pH of between 5.5 and 8. In another aspect of the present disclosure, a red blood cell composition comprises at least 2 red blood cells per 10 microliters of liquid. In another aspect, a red blood cell composition comprises at least 5, 10, 15, 20, 30, 40, 50, 60, 70, 80, 90, or 100 cells per 10 microliters of liquid.

The present disclosure also provides for, and includes, a method for increasing the shelf-life of a red blood cell composition for drug delivery comprising purging red blood cells comprising a pharmaceutical agent with carbon monoxide; and storing the purged red blood cells for a period of time. In an aspect of the present disclosure, the purging of red blood cells is with ambient air, carbon monoxide, N₂, or a combination thereof.

In an aspect of the present disclosure, the red blood cell composition comprising carboxyhemoglobin is stored for 1, 2, 3, 4, 5, 6, 7, 8, 9, or more weeks. In another aspect, the red blood cell composition is stored for between 1 and 3, 3 and 6, 6 and 9, or 9 and 12 weeks. In another aspect, the red blood cell composition is stored for at least 1, 2, 3, 4, 5, 6, 7, or 8 weeks.

In an aspect of the present disclosure, the red blood cell composition comprising cyano-methemoglobin is stored for 1, 2, 3, 4, 5, 6, 7, 8, 9, or more weeks. In another aspect, the red blood cell composition is stored for between 1 and 3, 3 and 6, 6 and 9, or 9 and 12 weeks. In another aspect, the red blood cell composition is stored for at least 1, 2, 3, 4, 5, 6, 7, or 8 weeks.

In an aspect of the present disclosure, the red blood cell composition comprising azido-methemoglobin is stored for 1, 2, 3, 4, 5, 6, 7, 8, 9, or more weeks. In another aspect, the red blood cell composition is stored for between 1 and 3, 3 and 6, 6 and 9, or 9 and 12 weeks. In another aspect, the red blood cell composition is stored for at least 1, 2, 3, 4, 5, 6, 7, or 8 weeks.

The present disclosure provides for, and includes, a pharmaceutical composition comprising a red blood cell expressing a pharmaceutical agent, wherein the pharmaceutical composition comprises a non-oxygen hemoglobin binding agent and less than 25% S02. In another aspect, the pharmaceutical composition comprises less than 20, 15, 10, 5, or 3% S02. In another aspect, the pharmaceutical composition comprises between 25 and 50, 50 and 75, or 75 and 100% carbon monoxide.

The present disclosure provides for, and includes, a storage vial comprising a carbon monoxide saturated pharmaceutical composition comprising a red blood cell comprising a pharmaceutical agent. In an aspect, the storage vial comprises a headspace with carbon monoxide, nitrogen, or argon. In an aspect, the storage vial comprises a headspace with carbon monoxide. In another aspect, the storage vial comprises a headspace with argon. In an aspect, the storage vial comprises a headspace with nitrogen.

The present disclosure also provides for, and includes, a method of packaging a predetermined dose of a red blood cell medication comprising: depleting oxygen by gas exchange with carbon monoxide; filing a medicament container with a predetermined dose of the red blood cell medication; and sealing the medicament container.

In an aspect of the present disclosure, a red blood cell medication comprises a pharmaceutical agent and a hemoglobin derivative selected from carbon monoxide, cyanide, or azide.

In an aspect of the present disclosure, a non-oxygen hemoglobin binding agent and a chemical binding agent is the same compound.

In an aspect of the present disclosure, a predetermined dose is between 1 and 100 microliters, 1 and 500 microliters, 500 and 1000 microliters, or 1 and 2 milliliters. In another aspect, the predetermined dose is at least 1, 10, 100, or 500 microliters.

In an aspect of the present disclosure, a red blood cell medication is a red blood cell comprising a pharmaceutical agent provided in the disclosure. In an aspect of the present disclosure, a medicament container is a vial, a syringe, a tube, a bag, or an ampule.

The present disclosure further provides for, and includes, a method for increasing the circulation life of a red blood cell for drug delivery comprising: obtaining a red blood cell comprising a pharmaceutical agent; treating the red blood cell with an agent to prepare a red blood cell comprising a hemoglobin derivative; and storing the red blood cell comprising the pharmaceutical agent under a storage atmosphere.

In an aspect of the present disclosure, the circulation life of a red blood cell in a patient in need thereof is increased by at least 2, 4, 5, 8, 16, or 32 days relative to a red blood cell comprising oxyhemoglobin and stored under similar conditions. In another aspect, the circulation life of a red blood cell for drug delivery is increased by at least 1, 2, or 3, weeks relative to a red blood cell comprising oxyhemoglobin and stored under similar conditions.

In an aspect of the present disclosure, “similar conditions” are defined by all conditions being matched except for oxygen saturation, hemoglobin derivative, or oxygen saturation and hemoglobin derivative. For example, similar conditions include length of time in storage, storage temperature, and storage container. In an aspect, similar conditions does not include the headspace gas composition (i.e., CO vs. ambient air). In an aspect, similar conditions include storage temperature, length of storage time, and additive solutions.

In an aspect of the present disclosure, “RBCs comprising oxyhemoglobin” comprises a mixture of oxy- and deoxy-hemoglobin with oxy-hemoglobin comprising greater than 50%. In another aspect, RBCs comprising oxyhemoglobin comprise greater than 60% oxyhemoglobin. In another aspect, RBCs comprising oxyhemoglobin comprise greater than 70% oxyhemoglobin. In another aspect, RBCs comprising oxyhemoglobin comprise greater than 80% oxyhemoglobin. In another aspect, RBCs comprising oxyhemoglobin comprise greater than 90% oxyhemoglobin. In another aspect, RBCs comprising oxyhemoglobin comprise greater than 95% oxyhemoglobin. In another aspect, RBCs comprising oxyhemoglobin comprise greater than 98%. In another aspect, oxy-hemoglobin comprises 100% oxyhemoglobin. In yet another aspect, RBCs comprising oxyhemoglobin comprise less than 50, 40, 30, 20, 10, 5, or 2% deoxy-hemoglobin.

As used herein, the term “reagent red blood cells” are red blood cells that have been antigenically characterized. In an aspect of the present disclosure, reagent red blood cells are red blood cells that have been antigenically characterized and stabilized with a hemoglobin derivative selected from the group consisting of carbon monoxide, cyanide, and azide. In another aspect, reagent red blood cells are packed red blood cells. In another aspect, reagent red blood cells comprise a 2-3% suspension of pooled washed red blood cells. In another aspect, reagent red blood cells comprise a 2-3% suspension of pooled washed red blood cells in Modified Alsever's Solution. In yet another aspect, reagent red blood cells comprise a preservation solution. In an aspect, a preservation solution comprises trisodium citrate, citric acid, dextrose, inosine, neomycin sulphate, chloramphenicol, or a combination thereof. In another aspect, a preservation solution comprises trisodium citrate, citric acid, dextrose, inosine, neomycin sulphate (0.103 grams/liter), chloramphenicol (0.349 grams/liter), or a combination thereof.

As used herein, the term “reducing”, “reduction”, “reduced”, “decreasing”, or “decreased” is meant to refer to a final amount lower than an initial amount or lower relative to a control sample. In an aspect, a control sample comprises RBCs comprising oxyhemoglobin and stored under similar conditions. In another aspect, a control sample comprises RBCs comprising a mixture of oxy- and deoxy-hemoglobin.

As used herein, the term “increasing” or “increased” is meant to refer to a final amount higher than an initial amount or higher relative to a control sample.

The present specification provides for, and includes, the following embodiments:

Embodiment 1. A method for preserving reagent red blood cells (RBC) comprising:
a) obtaining red blood cells;
b) flushing said red blood cells with a gas comprising carbon monoxide to prepare carbon monoxide saturated RBCs (CO-Hb RBCs); and
c) storing said CO-Hb RBCs under anaerobic conditions in the presence of carbon monoxide (CO), wherein surface antigens of said CO-Hb RBCs are stabilized.

Embodiment 2. The method of embodiment 1, wherein said gas does not comprise oxygen.

Embodiment 3. The method of embodiment 1, wherein said CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s.

Embodiment 4. The method of embodiment 3, wherein said CO-Hb RBCs are blood group O cells that further are positive for the surface antigens I, Lu^{a}, Lu^{b}, Js^{b}, Kp^{b}, and Yt^{a}.

Embodiment 5. The method of embodiment 4, wherein said CO-Hb RBCs are blood group O cells that are negative for the surface antigens Js^{a}, Kp^{a}, Wr^{a}, Di^{a}, V^{w}, V, Lu^{a}, and C^{w}.

Embodiment 6. The method of embodiment 1, wherein said CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b}.

Embodiment 7. The method of embodiment 1, wherein said CO-Hb RBCs are type-O cells that are positive for the Rh antigens D, C, and e.

Embodiment 8. The method of embodiment 7, wherein said CO-Hb RBCs are type-O cells that are positive for the surface antigens I, Lu^{b}, Js^{b}, Kp^{b}, and Yt^{a}.

Embodiment 9. The method of embodiment 8, wherein said CO-Hb RBCs are type-O cells that are negative for the surface antigens Js^{a}, Kp^{a}, Wr^{a}, Di^{a} V^{w}, V, Lu^{a} and C^{w}.

Embodiment 10. The method of embodiment 1, wherein said CO-Hb RBCs are type-A cells that are positive for the surface antigen A1.

Embodiment 11. The method of embodiment 10, wherein said CO-Hb RBCs are type-A cells that are negative for surface antigens D, C, and E.

Embodiment 12. The method of embodiment 1, wherein said CO-Hb RBCs are type-A cells that are positive for the surface antigen A2.

Embodiment 13. The method of embodiment 12, wherein said CO-Hb RBCs are type-A cells that are negative for surface antigens D, C, and E.

Embodiment 14. The method of embodiment 1, wherein said CO-Hb RBCs are type-B cells that are positive for the surface antigen B.

Embodiment 15. The method of embodiment 14, wherein said CO-Hb RBCs are type-B cells that are negative for surface antigens D, C, and E.

Embodiment 16. The method of embodiment 1, wherein said CO-Hb RBCs are type-O cells that are positive for the surface antigens D, C, and e and having the Rh phenotype R₁R₁; are positive for the surface antigens D, C^{w}, and e and having the Rh phenotype R₁^{w}R₁; are positive for the surface antigens D, c, and E and having the Rh phenotype R₂R₂; or are positive for the surface antigens d, c, and e and having the Rh phenotype rr.

Embodiment 17. The method of embodiment 16, wherein said CO-Hb RBCs are type-O cells that are positive for the surface antigens Lu^{b}, Js^{b}, Kp^{b}, and Yt^{a}.

Embodiment 18. The method of embodiment 16, wherein said CO-Hb RBCs are type-O cells that are negative for the surface antigens Js^{a}, Kp^{a}, Wr^{a}, Di^{a}, V^{w}, V, Lu^{a} and C^{w}.

Embodiment 19. The method of embodiment 1, wherein said CO-Hb RBCs are type-O cells that are positive for the surface antigens D, C, and e and having the Rh haplotype R₁ or are positive for the surface antigens D, c, and e and having the Rh haplotype R₂.

Embodiment 20. The method of embodiment 19, wherein said CO-Hb RBCs are type-O cells that are positive for the surface antigens Lu^{b}, Js^{b}, Kp^{b}, and Yt^{a}.

Embodiment 21. The method of embodiment 19, wherein said CO-Hb RBCs are type-O cells that are negative for the surface antigens Js^{a}, Kp^{a}, Wr^{a}, Di^{a}, Vw, V, Lu^{a} and C^{w}.

Embodiment 22. The method of embodiment 1, wherein said CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a}.

Embodiment 23. The method of embodiment 22, wherein said CO-Hb RBCs are type-O cells that are positive for binding of antibodies to D, C, E, c, e, f, Jk^{a}, Jk^{b}, Le^{a}, Le^{b}, P₁, I, IH, Vel, PP₁P^{k} and P antigens.

Embodiment 24. The method of embodiment 22, wherein said CO-Hb RBCs are type-O cells having reduced or absent binding of antibodies to Fy^{a}, Fy^{b}, S, s, M, N, Xg^{a}, Pr, Ch^{a}, Rg^{a}, and Yk^{a}.

Embodiment 25. The method of embodiment 1, wherein said CO-Hb RBCs are type-O cells that are positive for binding of antibodies to the D antigen and said CO-Hb RBCs are sensitized with anti-D(Rh₀) serum.

Embodiment 26. The method of embodiment 1, wherein said CO-Hb RBCs are type-A cells that are positive for binding of antibodies to the A₂ antigen.

Embodiment 27. The method of embodiment 1, wherein said CO-Hb RBCs are type-B cells that are positive for binding of antibodies to the B antigen and are negative for binding of anti-D(Rh₀) antibodies.

Embodiment 28. The method of embodiment 1, wherein said CO-Hb RBCs are type-A cells that are positive for binding of antibodies to the Aᵢ antigen and are negative for binding of anti-D(Rh₀) antibodies.

Embodiment 29. The method of embodiment 1, wherein said CO-Hb RBCs are type-AB cells that are positive for binding of antibodies to the A₁, B antigens and the Rh antigens d, c, and e of Rh blood group rr (dce/dec).

Embodiment 30. The method of embodiment 1, wherein said CO-Hb RBCs are type-O cells that are positive for binding of antibodies to the Rh antigens D, d, C, c, and e and having the Rh phenotype R₁r (DCe/dce).

Embodiment 31. The method of embodiment 1, wherein said CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b}.

Embodiment 32. The method of embodiment 31, wherein said CO-Hb RBCs are type-O cells that are positive for binding of antibodies to the Lu^{b}, Js^{b}, Kp^{b}, and Yt^{a} antigens.

Embodiment 33. The method of embodiment 31, wherein said CO-Hb RBCs are type-O cells that are negative for the binding of antibodies to the Js^{a}, Kp^{a}, Wr^{a}, Di^{a}, V^{w}, V, Lu^{a} and C^{w} antigens.

Embodiment 34. The method of embodiment 1, wherein said CO-Hb RBCs are type-O cells no profile in Resolve® Panel A instructions.

Embodiment 35. The method of embodiment 1, wherein said CO-Hb RBCs are type-O cells no profile in Resolve® Panel B instructions.

Embodiment 36. The method of embodiment 1, wherein said CO-Hb RBCs are type-O cells no profile in Resolve® Panel C instructions.

Embodiment 37. A kit comprising:
a) one or more vials of carbon monoxide saturated RBCs (CO-Hb RBCs), said vials comprising a buffer;
b) a plurality of CO-Hb RBCs having a common set of surface antigens selected from the group consisting of:
   (i) CO-Hb RBCs that are blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s;
   (ii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a};
   (iii). CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a} and negative for the surface antigens Js^{a}, Kp^{a}, Wr^{a}, Di^{a}, V^{w}, V, Lu^{a}, and C^{w};
   (iv) CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b}.
   (v) CO-Hb RBCs are type-O cells that are positive for the Rh antigens D, C, and e;
   (vi) CO-Hb RBCs are type-O cells that are positive for the Rh antigens D, C, and e, I, Lu^{b}, Js^{b}, Kp^{b}, and Yt^{a};
   (vii) CO-Hb RBCs are type-O cells that are positive for the Rh antigens D, C, and e, I, Lu^{b}, Js^{b}, Kp^{b}, and Yt^{a}, and that are negative for the surface antigens Js^{a}, Kp^{a}, Wr^{a}, Di^{a} V^{w}, V, Lu^{a} and C^{w};
   (viii) CO-Hb RBCs are type-A cells that are positive for the surface antigen A1;
   (ix) CO-Hb RBCs are type-A cells that are positive for the surface antigen A1 and are negative for surface antigens D, C, and E;
   (x) CO-Hb RBCs are type-A cells that are positive for the surface antigen A2;
   (xi) CO-Hb RBCs are type-A cells that are positive for the surface antigen A2; and are negative for surface antigens D, C, and E;
   (xii) CO-Hb RBCs are type-B cells that are positive for the surface antigen B;
   (xiii) CO-Hb RBCs are type-B cells that are positive for the surface antigen B and are negative for surface antigens D, C, and E;
   (xiv) CO-Hb RBCs are type-O cells that are positive for the surface antigens D, C, and e and having the Rh phenotype R₁R₁;
   (xv) CO-Hb RBCs are type-O cells that are positive for the surface antigens D, C^{w}, and e and having the Rh phenotype R₁^{w}R₁;
   (xvi) CO-Hb RBCs are type-O cells that are positive for the surface antigens D, c, and E and having the Rh phenotype R₂R₂;
   (xvii) CO-Hb RBCs are type-O cells that are positive for the surface antigens d, c, and e and having the Rh phenotype rr;
   (xviii) CO-Hb RBCs are type-O cells that are positive for the surface antigens D, C, and e and having the Rh phenotype R₁R₁ and are positive for the surface antigens Lu^{b}, Js^{b}, Kp^{b}, and Yt^{a};
   (xix) CO-Hb RBCs are type-O cells that are positive for the surface antigens D, C^{w}, and e and having the Rh phenotype R₁^{w}R₁ and are positive for the surface antigens Lu^{b}, Js^{b}, Kp^{b}, and Yt^{a};
   (xx) CO-Hb RBCs are type-O cells that are positive for the surface antigens D, c, and E and having the Rh phenotype R₂R₂ and are positive for the surface antigens Lu^{b}, Js^{b}, Kp^{b}, and Yt^{a};
   (xxi) CO-Hb RBCs are type-O cells that are positive for the surface antigens d, c, and e and having the Rh phenotype rr and are positive for the surface antigens Lu^{b}, Js^{b}, Kp^{b}, and Yt^{a};
   (xxii) CO-Hb RBCs are type-O cells that are positive for the surface antigens D, C, and e and having the Rh phenotype R₁R₁ and are negative for the surface antigens Js^{a}, Kp^{a}, Wr^{a}, Di^{a}, V^{w}, V, Lu^{a} and C^{w};
   (xxiii) CO-Hb RBCs are type-O cells that are positive for the surface antigens D, C^{w}, and e and having the Rh phenotype R₁^{w}R₁ and are negative for the surface antigens Js^{a}, Kp^{a}, Wr^{a}, Di^{a}, V^{w}, V, Lu^{a} and C^{w};
   (xxiv) CO-Hb RBCs are type-O cells that are positive for the surface antigens D, c, and E and having the Rh phenotype R₂R₂ and are negative for the surface antigens Js^{a}, Kp^{a}, Wr^{a}, Di^{a}, V^{w}, V, Lu^{a} and C^{w};
   (xxv) CO-Hb RBCs are type-O cells that are positive for the surface antigens d, c, and e and having the Rh phenotype rr and are negative for the surface antigens Js^{a}, Kp^{a}, Wr^{a}, Di^{a}, V^{w}, V, Lu^{a} and C^{w};
   (xxvi) CO-Hb RBCs are type-O cells that are positive for the surface antigens D, C, and e and having the Rh haplotype R₁ or are positive for the surface antigens D, c, and e and having the Rh haplotype R₂;
   (xxvii) CO-Hb RBCs are type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R² CO-Hb RBCs and are positive for the surface antigens Lu^{b}, Js^{b}, Kp^{b}, and Yt^{a};
   (xxviii) CO-Hb RBCs are type-O cells that are positive for the surface antigens D, C, and e and having the Rh haplotype R₁ and are negative for the surface antigens Js^{a}, Kp^{a}, Wr^{a}, Di^{a}, V^{w}, V, Lu^{a} and C^{w} or are positive for the surface antigens D, c, and e and having the Rh haplotype R₂ and are negative for the surface antigens Js^{a}, Kp^{a}, Wr^{a}, Di^{a}, V^{w}, V, Lu^{a} and C^{w};
   (xxix) CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a};
   (xxx) CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens;
   (xxxi) CO-Hb RBCs are type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a};
   (xxxii) CO-Hb RBCs are type-O cells that are positive for binding of antibodies to the D antigen and said CO-Hb RBCs are sensitized with anti-D(Rh₀) serum;
   (xxxiii) CO-Hb RBCs are type-A cells that are positive for binding of antibodies to the A₂ antigen;
   (xxxiv) CO-Hb RBCs are type-B cells that are positive for binding of antibodies to the B antigen and are negative for binding of anti-D(Rh₀) antibodies;
   (xxxv) CO-Hb RBCs are type-A cells that are positive for binding of antibodies to the Aᵢ antigen and are negative for binding of anti-D(Rh₀) antibodies;
   (xxxvi) CO-Hb RBCs are type-AB cells that are positive for binding of antibodies to the A₁, B antigens and the Rh antigens d, c, and e of Rh blood group rr (dce/dec);
   (xxxvii) CO-Hb RBCs are type-O cells that are positive for binding of antibodies to the Rh antigens D, d, C, c, and e and having the Rh phenotype R₁r (DCe/dce);
   (xxxviii) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b};
   (xxxix) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens;
   (xl) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, V^{w}, V, Lu^{a} and C^{w} antigens; and c) instructions.

Embodiment 38. A vial of carbon monoxide saturated RBCs (CO-Hb RBCs) comprising a buffer and CO-Hb RBCs selected from the group consisting of:
(i) CO-Hb RBCs that are blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s;
(ii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a};
(iii). CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a} and negative for the surface antigens Js^{a}, Kp^{a}, Wr^{a}, Di^{a}, V^{w}, V, Lu^{a}, and C^{w};
(iv) CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b};
(v) CO-Hb RBCs are type-O cells that are positive for the Rh antigens D, C, and e;
(vi) CO-Hb RBCs are type-O cells that are positive for the Rh antigens D, C, and e, I, Lu^{b}, Js^{b}, Kp^{b}, and Yt^{a};
(vii) CO-Hb RBCs are type-O cells that are positive for the Rh antigens D, C, and e, I, Lu^{b}, Js^{b}, Kp^{b}, and Yt^{a}, and that are negative for the surface antigens Js^{a}, Kp^{a}, Wr^{a}, Di^{a} V^{w}, V, Lu^{a} and C^{w};
(viii) CO-Hb RBCs are type-A cells that are positive for the surface antigen A1;
(ix) CO-Hb RBCs are type-A cells that are positive for the surface antigen A1 and are negative for surface antigens D, C, and E;
(x) CO-Hb RBCs are type-A cells that are positive for the surface antigen A2;
(xi) CO-Hb RBCs are type-A cells that are positive for the surface antigen A2; and are negative for surface antigens D, C, and E;
(xii) CO-Hb RBCs are type-B cells that are positive for the surface antigen B;
(xiii) CO-Hb RBCs are type-B cells that are positive for the surface antigen B and are negative for surface antigens D, C, and E;
(xiv) CO-Hb RBCs are type-O cells that are positive for the surface antigens D, C, and e and having the Rh phenotype R₁R₁;
(xv) CO-Hb RBCs are type-O cells that are positive for the surface antigens D, C^{w}, and e and having the Rh phenotype R₁^{w}R₁;
(xvi) CO-Hb RBCs are type-O cells that are positive for the surface antigens D, c, and E and having the Rh phenotype R₂R₂;
(xvii) CO-Hb RBCs are type-O cells that are positive for the surface antigens d, c, and e and having the Rh phenotype rr;
(xviii) CO-Hb RBCs are type-O cells that are positive for the surface antigens D, C, and e and having the Rh phenotype R₁R₁ and are positive for the surface antigens Lu^{b}, Js^{b}, Kp^{b}, and Yt^{a};
(xix) CO-Hb RBCs are type-O cells that are positive for the surface antigens D, C^{w}, and e and having the Rh phenotype R₁^{w}R₁ and are positive for the surface antigens Lu^{b}, Js^{b}, Kp^{b}, and Yt^{a};
(xx) CO-Hb RBCs are type-O cells that are positive for the surface antigens D, c, and E and having the Rh phenotype R₂R₂ and are positive for the surface antigens Lu^{b}, Js^{b}, Kp^{b}, and Yt^{a};
(xxi) CO-Hb RBCs are type-O cells that are positive for the surface antigens d, c, and e and having the Rh phenotype rr and are positive for the surface antigens Lu^{b}, Js^{b}, Kp^{b}, and Yt^{a};
(xxii) CO-Hb RBCs are type-O cells that are positive for the surface antigens D, C, and e and having the Rh phenotype R₁R₁ and are negative for the surface antigens Js^{a}, Kp^{a}, Wr^{a}, Di^{a}, V^{w}, V, Lu^{a} and C^{w};
(xxiii) CO-Hb RBCs are type-O cells that are positive for the surface antigens D, C^{w}, and e and having the Rh phenotype R₁^{w}R₁ and are negative for the surface antigens Js^{a}, Kp^{a}, Wr^{a}, Di^{a}, V^{w}, V, Lu^{a} and C^{w};
(xxiv) CO-Hb RBCs are type-O cells that are positive for the surface antigens D, c, and E and having the Rh phenotype R₂R₂ and are negative for the surface antigens Js^{a}, Kp^{a}, Wr^{a}, Di^{a}, V^{w}, V, Lu^{a} and C^{w};
(xxv) CO-Hb RBCs are type-O cells that are positive for the surface antigens d, c, and e and having the Rh phenotype rr and are negative for the surface antigens Js^{a}, Kp^{a}, Wr^{a}, Di^{a}, V^{w}, V, Lu^{a} and C^{w};
(xxvi) CO-Hb RBCs are type-O cells that are positive for the surface antigens D, C, and e and having the Rh haplotype R₁ or are positive for the surface antigens D, c, and e and having the Rh haplotype R₂;
(xxvii) CO-Hb RBCs are type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R₂ CO-Hb RBCs and are positive for the surface antigens Lu^{b}, Js^{b}, Kp^{b}, and Yt^{a};
(xxviii) CO-Hb RBCs are type-O cells that are positive for the surface antigens D, C, and e and having the Rh haplotype R₁ and are negative for the surface antigens Js^{a}, Kp^{a}, Wr^{a}, Di^{a}, V^{w}, V, Lu^{a} and C^{w} or are positive for the surface antigens D, c, and e and having the Rh haplotype R₂ and are negative for the surface antigens Js^{a}, Kp^{a}, Wr^{a}, Di^{a}, V^{w}, V, Lu^{a} and C^{w};
(xxix) CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a};
(xxx) CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens;
(xxxi) CO-Hb RBCs are type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a};
(xxxii) CO-Hb RBCs are type-O cells that are positive for binding of antibodies to the D antigen and said cells are sensitized with anti-D(Rh₀) serum;
(xxxiii) CO-Hb RBCs are type-A cells that are positive for binding of antibodies to the A2 antigen;
(xxxiv) CO-Hb RBCs are type-B cells that are positive for binding of antibodies to the B antigen and are negative for binding of anti-D(Rh₀) antibodies;
(xxxv) CO-Hb RBCs are type-A cells that are positive for binding of antibodies to the Aᵢ antigen and are negative for binding of anti-D(Rh₀) antibodies;
(xxxvi) CO-Hb RBCs are type-AB cells that are positive for binding of antibodies to the A₁, B antigens and the Rh antigens d, c, and e of Rh blood group rr (dce/dec);
(xxxvii) CO-Hb RBCs are type-O cells that are positive for binding of antibodies to the Rh antigens D, d, C, c, and e and having the Rh phenotype R₁r (DCe/dce);
(xxxviii) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b};
(xxxix) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens; and
(xl) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, V^{w}, V, Lu^{a} and C^{w} antigens.

Embodiment 39. A method for preserving reagent red blood cells (RBC) comprising:
a) obtaining red blood cells that have been antigenically characterized;
b) treating said red blood cell with a chemical agent to prepare a red blood cell comprising a hemoglobin derivative;
c) and storing said red blood cells comprising a hemoglobin derivative under anaerobic conditions to form reagent red blood cells, wherein surface antigens of said reagent red blood cells comprising a hemoglobin derivative are stabilized.

Embodiment 40. The method of embodiment 39, wherein said chemical agent is carbon monoxide (CO) and said hemoglobin derivative is carboxy-hemoglobin.

Embodiment 41. The method of embodiment 39, wherein said chemical agent is cyanide and said hemoglobin derivative is cyano-methemoglobin.

Embodiment 42. The method of embodiment 39, wherein said chemical agent is azide (N₃) and said hemoglobin derivative is azido-methemoglobin prepared by reacting with an aqueous solution of sodium azide.

Embodiment 43. The method of embodiment 39, further comprising characterizing red blood cells.

Embodiment 44. A method for increasing the shelf-life of a red blood cell composition for drug delivery comprising:
obtaining a red blood cell comprising a pharmaceutical agent;
treating said red blood cell with a chemical agent to prepare a red blood cell comprising a hemoglobin derivative; and
storing said red blood cell comprising said pharmaceutical agent under a storage atmosphere.

Embodiment 45. The method of embodiment 44, wherein said storage atmosphere comprises less than 10 mmHg oxygen.

Embodiment 46. The method of embodiment 44, wherein said storage is under ambient pressure.

Embodiment 47. The method of embodiment 44, wherein said chemical agent is carbon monoxide (CO) and said hemoglobin derivative is carboxy-hemoglobin.

Embodiment 48. The method of embodiment 44, wherein said chemical agent is cyanide and said hemoglobin derivative is cyano-methemoglobin.

Embodiment 49. The method of embodiment 44, wherein said chemical agent is azide (N₃) and said hemoglobin derivative is azido-methemoglobin prepared by reacting with an aqueous solution of sodium azide.

Embodiment 50. The method of embodiment 47, wherein said storage atmosphere comprises carbon monoxide.

Embodiment 51. The method of embodiment 48, wherein said atmosphere comprises wherein said storage atmosphere comprises ambient air, carbon monoxide, N₂, or mixture thereof.

Embodiment 52. The method of embodiment 49, wherein said atmosphere comprises nitrogen.

Embodiment 53. The method of embodiment 47, wherein said shelf-life is increased by one or more weeks relative to RBCs comprising oxyhemoglobin and stored under similar conditions.

Embodiment 54. The method of embodiment 53, wherein said RBCs comprising oxyhemoglobin comprises a mixture of oxy- and deoxy-hemoglobin with oxy-hemoglobin comprising greater than 50%.

Embodiment 55. The method of embodiment 54, wherein said RBCs comprising oxyhemoglobin comprise greater than, 60, 70, 80, 90, or 95% oxyhemoglobin.

Embodiment 56. The method of embodiment 48, wherein said shelf-life is increased by one or more weeks relative to RBCs comprising oxyhemoglobin and stored under similar conditions.

Embodiment 57. The method of embodiment 49, wherein said shelf-life is increased by one or more weeks relative to RBCs comprising oxyhemoglobin and stored under similar conditions.

Embodiment 58. The method of any one of embodiments 47 to 49, wherein said shelf-life is increased by one or more days relative to RBCs comprising oxyhemoglobin and stored under similar conditions.

Embodiment 59. The method of any one of embodiments 45 to 58, wherein the growth of Heinz bodies in said red blood cells is reduced relative to RBCs comprising oxyhemoglobin and stored under similar conditions.

Embodiment 60. The method of any one of embodiments 45 to 58, wherein cell lysis is reduced in said red blood cell composition relative to RBCs comprising oxyhemoglobin and stored under similar conditions.

Embodiment 61. The method of embodiment 44, wherein said pharmaceutical agent is a transgene expressed on the cell surface of said RBC.

Embodiment 62. The method of embodiment 44, wherein said pharmaceutical agent is localized in the cytosol of said RBC.

Embodiment 63. The method of embodiment 44, wherein said pharmaceutical agent is localized to the cell surface of said RBC.

Embodiment 64. The method of embodiment 44, wherein said pharmaceutical agent is localized to the intracellular membrane.

Embodiment 65. The method of embodiment 44, wherein said treating comprises gas exchange.

Embodiment 66. The method of embodiment 65, where said gas exchange is rapid gas exchange, overnight gas exchange, membrane gas exchange, or microbubble gas exchange.

Embodiment 67. The method of embodiment 65 or 66, wherein said gas exchange is with carbon monoxide.

Embodiment 68. The method of embodiment 65 or 66, wherein said gas exchange is with cyanide by treatment with HCN.

Embodiment 69. The method of embodiment 65 or 66, wherein said gas exchange is by treating red blood cells with sodium azide (NaN₃) solution.

Embodiment 70. The method of embodiment 44, wherein said pharmaceutical agent is a fusion protein.

Embodiment 71. The method of embodiment 70, wherein said fusion protein is selected from the group consisting of those listed in Table 4 and Table 5.

Embodiment 72. The method of embodiment 44, wherein said pharmaceutical agent is a protein selected from the classes of proteins listed in Table 11.

Embodiment 73. The method of embodiment 44, wherein said pharmaceutical agent is selected from the pharmaceuticals listed in Table 12.

Embodiment 74. The method of embodiment 44, wherein said storing is at a temperature between 0.1 and 6° C.

Embodiment 75. The method of embodiment 44, wherein said storing is at a temperature greater than 6° C.

Embodiment 76. The method of embodiment 44, wherein said storing is at a temperature between 24 and 38° C.

Embodiment 77. The method of embodiment 44, wherein said storing is at 37° C.

Embodiment 78. The method of embodiment 44, further comprising mixing red blood cells with an additive solution having a pH of between 5.5 and 8.

Embodiment 79. The method of embodiment 78, wherein said additive solution comprises one or more of glucose, phosphate, citrate, bicarbonate, or sodium chloride (NaCl).

Embodiment 80. The method of embodiment 44, wherein said red blood cell composition comprises at least 100 red blood cells per microliter. A method for increasing the shelf-life of a red blood cell composition for drug delivery comprising: purging red blood cells comprising a pharmaceutical agent with carbon monoxide; and storing said purged red blood cells for a period of time.

Embodiment 81. The method of embodiment 80, wherein said shelf-life is increased by more than one week relative to RBCs comprising oxyhemoglobin and stored under similar conditions.

Embodiment 82. The method of embodiment 80, wherein said shelf-life is increased by one or more days relative to RBCs comprising oxyhemoglobin and stored under similar conditions.

Embodiment 83. The method of embodiment 80, wherein said storing is under reduced oxygen conditions of less than 25% oxygen.

Embodiment 84. The method of embodiment 80, wherein said pharmaceutical agent is one or more expressed proteins.

Embodiment 85. The method of embodiment 80, wherein said pharmaceutical agent is localized in the cytosol of said RBC.

Embodiment 86. The method of embodiment 80, wherein said pharmaceutical agent is localized to the cell surface of said RBC.

Embodiment 87. The method of embodiment 80, wherein said pharmaceutical agent is localized to the RBC membrane.

Embodiment 88. The method of embodiment 80, wherein said purging is by rapid, overnight, or gas exchange with carbon monoxide.

Embodiment 89. The method of embodiment 80, wherein said storing is at 37° C.

Embodiment 90. The method of embodiment 80, wherein said storing is at a temperature between 0.1 and 6° C.

Embodiment 91. The method of embodiment 80, wherein said storing is at a temperature greater than 6° C.

Embodiment 92. The method of embodiment 80, further comprising mixing red blood cells with an additive solution having a pH of between 5.5 and 8.

Embodiment 93. The method of embodiment 92, wherein said additive solution comprises one or more of glucose, phosphate, citrate, bicarbonate, or sodium chloride (NaCl).

Embodiment 94. The method of embodiment 80, wherein said red blood cell composition comprises at least 100 red blood cells per microliter.

Embodiment 95. The method of embodiment 44, wherein said headspace atmosphere comprises less than 5 mmHg oxygen.

Embodiment 96. A pharmaceutical composition comprising a red blood cell expressing a pharmaceutical agent, wherein said pharmaceutical composition comprises a non-oxygen hemoglobin binding agent and less than 25% SO₂.

Embodiment 97. The pharmaceutical composition of embodiment 96, wherein said non-oxygen hemoglobin binding agent is carbon monoxide.

Embodiment 98. The method of embodiment 96, wherein said non-oxygen hemoglobin binding agent is cyanide.

Embodiment 99. The method of embodiment 96, wherein said non-oxygen hemoglobin binding agent is azide (N₃).

Embodiment 100. A storage vial comprising a carbon monoxide saturated pharmaceutical composition comprising a red blood cell comprising a pharmaceutical agent.

Embodiment 101. The pharmaceutical composition of embodiment 100, wherein said pharmaceutical agent comprises an antigen expressed by fusion to a red blood cell protein, selected from the group consisting of those listed in Table 5, Table 6, and Table 7.

Embodiment 102. The pharmaceutical composition of embodiment 100, wherein said pharmaceutical agent is a protein selected from the class of protein listed in Table 11.

Embodiment 103. The pharmaceutical composition of embodiment 101, wherein said antigen is selected from the antigens listed in Table 8, Table 9, or Table 10.

Embodiment 104. The pharmaceutical composition of embodiment 101, wherein said pharmaceutical agent comprises an antibody molecule.

Embodiment 105. The pharmaceutical composition of embodiment 101, wherein said pharmaceutical agent is an agent that inhibits an immune checkpoint molecule.

Embodiment 106. The pharmaceutical composition of embodiment 101, wherein said antigen is selected from the antigens of Table 12.

Embodiment 107. The pharmaceutical composition of embodiment 104, wherein said antibody is selected from the antibodies listed in Table 17.

Embodiment 108. A method of packaging a predetermined dose of a red blood cell medication comprising: depleting oxygen by gas exchange with carbon monoxide; filling a medicament container with a predetermined dose of said red blood cell medication; and sealing said medicament container.

Embodiment 109. The method of embodiment 108, wherein said packaging steps are performed under oxygen reduced conditions.

Embodiment 110. A method for increasing the in vivo circulation time of a red blood cell for drug delivery comprising: obtaining a red blood cell comprising a pharmaceutical agent; treating said red blood cell with a chemical agent to prepare a red blood cell comprising a hemoglobin derivative; and storing said red blood cell comprising said pharmaceutical agent under a storage atmosphere.

Embodiment 111. The method of embodiment 110, wherein said chemical agent is cyanide and said hemoglobin derivative is cyano-hemoglobin.

Embodiment 112. The method of embodiment 110, wherein said chemical agent is azide (N₃) and said hemoglobin derivative is azido-methemoglobin prepared by reacting said red blood cells with an aqueous solution of sodium azide.

Embodiment 113. The method of embodiment 110, wherein said in vivo circulation time is increased by at least 5 days relative to RBCs comprising oxyhemoglobin and stored under similar conditions.

Having now generally described the invention, the same will be more readily understood through reference to the following examples that are provided by way of illustration, and are not intended to be limiting of the present invention, unless specified.

Each periodical, patent, and other document or reference cited herein is herein incorporated by reference in its entirety.

### EXAMPLES

### Example 1: Collection of Blood and Preparation of Red Cell Concentrate (RCC)

Blood for the preparation of Reagent RBCs is collected from an established donor into anti-coagulant solution using standard methods. Various known anticoagulants suitable for use in transfusion medicine are suitable including Citrate Phosphate Dextrose (CPD) and Acid Citrate Dextrose (ACD), but other anticoagulants such as ethylenediaminetetraacetic acid (EDTA) and ethylene glycol-bis(β-aminoethyl ether)-N,N,N′,N′-tetraacetic acid (EGTA) can be used as appropriate if the blood will not be used for transfusion. Collected blood is subjected to centrifugation or filtration to separate the white blood cells (WBC) and excess plasma to prepare packed red blood cells (pRBC) or Red Cell Concentrate (RCC).

### Example 2: Preparation of Hb-CO Containing RCC (Hb-CO RCC)

Preferably without delay, the red cell concentrate (RCC) prepared in Example 1 is converted to Hb-CO by one of the following methods:

a. Rapid Gas Exchange:

RCC is held in a polyvinyl chloride or other suitable bag (150 ml or more) and carbon monoxide is introduced and the bag containing the RCC and CO are placed on a platelet shaker for about 10 minutes. After incubation, the gas containing un-exchanged CO, released and residual oxygen, and carbon dioxide is expressed and replaced with fresh CO. After a second incubation of a platelet shaker for about 10 minutes, the gas is expressed a second time and replaced with a third volume of CO. Following a final incubation with shaking on a platelet shaker, the excess gas is removed, and the Hb-CO RCC transferred under anaerobic conditions to suitable vials for further characterization, quality control, and storage.

b. Overnight Gas Exchange:

RCC is held in a polyvinyl chloride or other suitable bag (150 ml or more) and carbon monoxide is introduced and the bag containing the RCC and CO are placed on overnight at 4° C. with constant gentle shaking or at agitated at regular intervals (e.g., 3 to 5× over 8 hours). After incubation, the CO containing excess gas is removed and the Hb-CO RCC transferred under anaerobic conditions to suitable vials for further characterization, quality control, and storage.

c. Membrane Gas Exchange

RCC held in a polyvinyl chloride or other suitable bag (150 ml or more) is pumped through a Sorin D100 oxygenator with carbon monoxide as the source gas. The RCC is pumped through the D100 using either a centrifugal or peristaltic pump for 30 minutes. Oxygen and CO levels are monitored until Hb-CO levels of greater than 95% are achieved. Alternatively, with the use of mixed gas of CO and CO₂, pH of the suspension is optimized.

d. Microbubble Gas Exchange:

RCC is held in a polyvinyl chloride or other suitable vented bag (150 ml or more) and carbon monoxide is bubbled through the RCC. Care is taken to ensure that the bubbles are no more than 1 μm in diameter to prevent lysis of the red blood cells. The resulting Hb-CO RCC is transferred under anaerobic conditions to suitable vials for further characterization, quality control, and storage. Alternatively, with the use of mixed gas of CO and CO₂, pH of the suspension is optimized.

e. Modified HEMANEXT® Oxygen Reduction Bag (ORB)

A HEMANEXT® Oxygen Reduction Bag (ORB) as described in U.S. Pat. No. 10,058,091, issued Aug. 28, 2018, is modified to remove the sorbent pack and the headspace filled with CO gas (100 to 200 ml). The CO containing ORB bag is agitated at room temperature on a platelet shaker for 30 minutes. Alternatively, the CO containing ORB bag is placed at 4° C. with either constant shaking or with agitation at regular intervals (e.g., 3 to 5× over 8 hours).

### Example 3: Preparation and Packaging of HB—CO Reagent RBCs

Continue further manufacturing process with CO-treated RBC.

Package reagent RBC in a reagent bottle head and fill the head space CO under positive pressure and store at 4° C.

### Example 4: Long-Term Incubation of RBCs at 37° C. with Carbon Monoxide

A preliminary study is undertaken in an attempt to estimate the behavior of stored RBCs after transfusion into a recipient. The experiment is set up to incubate fresh and stored RBCs in a tissue culture media at 37° C. for extended time, with RBC morphology as the outcome measure. When RBCs are placed in culture medium under ambient air, within 1-3 days, small dark nodules on the surface (Heinz body) appear and continue to grow in size over days. Within a week, Heinz bodies continue to grow and become large (estimated to be more than 1 um in diameter), resulting in the rupture of most of the RBCs. The result is the development of RBC ghosts (clear cytosol, with little or no hemoglobin) with attached large dark nodules.

Without being limited by theory, the development of Heinz bodies and RBC ghosts is likely caused by hemoglobin oxidation products. For example, ferrous (+2) iron in hemoglobin becomes oxidized to a ferric (+3) state by reacting with oxygen during incubation. When hemoglobin is oxidized to methemoglobin, it becomes unstable, and readily degrades into hemichromes, then to globin and hemin, which are all hydrophobic and precipitate to RBC membrane forming aggregates (Heinz body). Additionally, these hemoglobin oxidation products bind to proteins and RBC cytoskeleton disrupting normal morphology and functions. Normally in circulation, small Heinz bodies are removed by macrophages, and normal RBC morphology is maintained albeit with a reduced cell size. In combination with optimized nutrients and mechanical deformation in circulation, RBCs have circulation life of ˜120 days. In culture media without macrophages, growth of Heinz body was uninhibited, resulting in quick cell destruction.

Stabilization of hemoglobin with carbon monoxide (Hb-CO complex) prevents hemoglobin oxidation and inhibits the formation of Heinz bodies and cell destruction. Hb-CO complex RBCs maintain normal biconcave morphology over 2-3 weeks when ambient air is purged with 100% carbon monoxide or 5% CO₂/95% CO in the culture bottle during incubation at 37° C. cell culture environment.

1.-5. (canceled)

6. A method for increasing the shelf-life of a red blood cell composition for drug delivery comprising:
obtaining a red blood cell comprising a pharmaceutical agent;
treating said red blood cell comprising said pharmaceutical agent with a chemical agent to prepare a red blood cell comprising a hemoglobin derivative; and
storing said red blood cell comprising said hemoglobin derivative under a storage atmosphere to prepare a stored red blood cell composition for drug delivery.

7. The method of claim 6, wherein said storage atmosphere comprises an oxygen pressure of less than 10 millimeters of mercury (mmHg).

8. The method of claim 6, wherein said storing is under ambient pressure.

9. The method of claim 6, wherein
said chemical agent is carbon monoxide (CO) and said hemoglobin derivative is carboxy-hemoglobin (CO-Hb);
said chemical agent is cyanide and said hemoglobin derivative is cyano-methemoglobin (CN-Hb); or
said chemical agent is azide (N₃) and said hemoglobin derivative is azido-methemoglobin (N₃-Hb), wherein said N₃-Hb is prepared by reacting said red blood cell with an aqueous solution of sodium azide (NaN₃).

10. (canceled)

11. (canceled)

12. (canceled)

13. The method of claim 9, wherein said storage atmosphere comprises ambient air, carbon monoxide, nitrogen (N₂), or any mixture thereof.

14. (canceled)

15. The method of claim 9, wherein said shelf-life is increased by one or more weeks relative to a red blood cell composition comprising said pharmaceutical agent and oxyhemoglobin, and having been stored under similar conditions.

16. A pharmaceutical composition comprising a red blood cell expressing a pharmaceutical agent, wherein said pharmaceutical composition comprises a non-oxygen hemoglobin binding agent and less than 25% oxygen saturation (SO2).

17. (canceled)

18. A method of packaging a predetermined dose of a red blood cell medication comprising: depleting oxygen in said red blood cell medication by gas exchange with carbon monoxide; filling a medicament container with a predetermined dose of said red blood cell medication; and sealing said medicament container.

19. (canceled)

20. (canceled)

21. The method of claim 6, wherein the growth of Heinz bodies in said red blood cell is reduced relative to a red blood cell comprising and pharmaceutical agent and oxyhemoglobin, and having been stored under similar conditions.

22. The method of claim 6, wherein cell lysis in said red blood cell composition is reduced relative to a red blood cell composition comprising said pharmaceutical composition and oxyhemoglobin, and having been stored under similar conditions.

23. The method of claim 6, wherein said pharmaceutical agent is expressed on the cell surface of said red blood cell, localized in the cytosol of said red blood cell, localized on the cell surface of said red blood cell, or localized in the intracellular membrane of said red blood cell.

24. The method of claim 6, wherein said treating comprises gas exchange selected from the group consisting of rapid gas exchange, overnight gas exchange, membrane gas exchange, and microbubble gas exchange.

25. The method of claim 24, wherein said gas exchange comprises treating said red blood cell with carbon monoxide, treating said red blood cell with cyanide by treatment with hydrogen cyanide (HCN), or treating said red blood cell with a solution of sodium azide (NaN₃).

26. The method of claim 6, wherein said pharmaceutical agent is a fusion protein selected from the group consisting of cluster of differentiation 1 (CD1) to CD10, CD11a, CD11b, CD11c, CD12w, CD13 to CD48, CD49a, CD49b, CD49c, CD49d, CD49e, CD49f, CD53 to CD59, CD61, CD62E, CD62L, CD62P, CD63, CD68, CD69, CD71 to CD74, CD80 to CD83, CD86 to CD91, CD95, CD96, CD100, CD103, CD105 to CD107, CD107a, CD107b, CD109, CD117, CD120, CD122, CD127, CD132 to CD135, CD138, CD141 to CD144, CD147, CD151, CD152, CD154, CD156, CD158, CD163, CD165, CD166, CD168, CD184, CD186, CD195, CD197, CD199, CD209, CD202a, CD220, CD221, CD235a, CD271, CD279, CD303, CD304, CD326, Toll-like receptor 1 (TLR1), TLR2, TLR4, TLR5, TLR6, 2′,3′-cyclic-nucleotide 3′-phosphodiesterase acetylcholinesterase, actin alpha and beta chains, adenosine deaminase, adducin alpha subunit, aldolase A, ankyrin-1, ankyrin 1 isoform 2, ankyrin 1 isoform 4, ankyrin 1 splice form 2, ankyrin-3, aquaporin 1, aquaporin 7, arginase type 1, arginase type 1 erythroid variant, ATP-binding cassette half-transporter, ATP-binding cassette subfamily C member 6, ATP-binding cassette sub-family B member 6, mitochondrial band 3 anion transport protein, bA421I18.2, basal cell adhesion molecule protein, block of proliferation 1, C-1-tetrahydrofolate synthase, calcium transposing ATPase 4, channel-like integral membrane protein, complement receptor 1, adipocyte plasma membrane-associated protein, ammonium transporter Rh type A, basigin, equilibrative nucleoside transporter 1, erythroid membrane-associated protein, flotillin-1, flotillin-2, glucose transporter type I, glycophorin-A, glycophorin-B, glycophorin-C, immunoglobulin-like domain-containing receptor 1, integrin alpha-X, integrin beta-I, Kell blood group glycoprotein, large neutral amino acids transporter small subunit 3, membrane transport protein XK, membrane-associated progesterone receptor component 2, monocarboxylate transporter I, multidrug resistance-associated protein 4, neutral cholesterol ester hydrolase 1, plasma membrane calcium-transporting ATPase I, plasma membrane calcium-transporting ATPase 3, plasma membrane calcium-transporting ATPase 4, probable E3 ubiquitin-protein ligase C12orf51, Rh blood group CcEe antigen, Rh blood group SLC43A3 antigen, sodium/calcium exchanger SCL8A3, sodium/potassium-transporting ATPase subunit alpha-1, sodium/potassium-transporting ATPase subunit beta-3, creatine kinase, DC 38, duodenal cytochrome b, enhancer protein, far upstream element binding protein, glucose transporter glycoprotein, glutathione transferase, glyceraldehyde-3-phosphate dehydrogenase, glycophorin A, glycophorin A precursor, glycophorin C isoform 1, hemoglobin alpha, hemoglobin beta, hemoglobin delta, hemoglobin epsilon, hemoglobin gamma, human growth and transformation-dependent protein, Hypothetical protein XP_061743, Hypothetical protein XP_089854, Hypothetical protein XP_091430, Hypothetical protein XP_091724, Hypothetical protein XP_092517, Hypothetical protein XP_095819, stomatin, stomatin-like protein, thioredoxin-related transmembrane protein 4, transmembrane and coiled-coil domain family 2, transferrin receptor protein 1, urea transporter 1, zinc transporter I, 55 kilodalton (kDa) erythrocyte membrane protein, actin alpha cardiac muscle, actin cytoplasmic, actin-related protein 2, actin-related protein ⅔ complex subunit 1B, actin-related protein ⅔ complex subunit 2, actin-related protein 3, alpha-actinin-4, alpha-adducin, beta-actin-like protein 2, beta-adducin, capping protein (actin filament) muscle Z-line beta, cortactin, dynactin 2 (P50) isoform CRA_b, erythrocyte membrane protein band 4.2, filamin-A, gamma-adducin, gelsolin, kinesin-1 heavy chain, microtubule-associated protein RP/EB family member 1, myosin light chain 4, myosin light polypeptide 6, myosin heavy chain 11 smooth muscle, myosin-9, myosin-10, myosin-14, Hypothetical protein XP_100510, Hypothetical protein XP_100619, Hypothetical protein XP 100665, Hypothetical protein XP_100925, Hypothetical protein XP_103707, Hypothetical protein XP_106269, Ig heavy chain V-V region, KIAA0340, KIAA1741 protein, Lyn B protein, membrane protein p55, phosphatidylinositol-4-phosphate 5 kinase type III, phosphoribosyl pyrophosphate synthetase, poly (A)-specific ribonuclease, presenilin-associated protein, protein band 3, protein band 4.1, protein band 4.1 (elliptocytosis 1, RH-linked), protein band 4.2, protein band 4.9 (dematin), protein band 7.2b (stomatin), Ras-related protein 35 (RAB35), rabphilin-3 A-integrating protein, Ra1 A binding protein, protein 4.1, spectrin alpha chain erythrocyte, spectrin beta chain erythrocyte, talin-1, talin-2, tropomodulin-1, tropomyosin I (alpha) isoform 4, tropomyosin 3, tropomyosin alpha-3 chain, tubulin alpha-I chain, tubulin beta chain, tubulin alpha 1 (testis specific), tubulin alpha 8, tubulin beta 6, vinculin, 78 kilodalton (kDa) glucose-regulated protein, antigen KI-67, ATP-dependent RNA helicase DDX39A, calnexin, calreticulin, DNA topoisomerase 1, DNA-dependent protein kinase catalytic subunit, dolichyl-diphosphooligosaccharide-protein glycosyltransferase 48 kilodalton (kDa) subunit, dolichyl-diphosphooligosaccharide-protein glycosyltransferase subunit 1, endoplasmic reticulum resident protein 29, endoplasmic reticulum resident protein 44, endoplasmin, ER-Golgi SNARE of 24 kildoaltons (kDa), FACT complex submit SPT16, glucosidase 2 subunit beta, heme oxygenase 1, hemogen, heterochromatin protein 1-binding protein 3, high mobility group protein BI, high mobility group protein B2, Ras-related protein 1A (RAP1A), Ras-related protein 1B (RAP1B), Ras-related protein 2B (RAP2B), Rh blood D group antigen polypeptide, Rhesus D category VI type III protein, solute carrier family 29 (nucleoside transporter) member 1, solute carrier family 2 (facilitated glucose transporter) member 1, spectrin alpha chain, spectrin beta chain, translation initiation factor 2C, tropomodulin, tropomyosin 3, tropomyosin isoform, tropomyosin alpha chain (smooth muscle) 26, vesicle-associated membrane protein 2 (synaptobrevin 2), zona pellucida binding protein, histone H1.1, histone H2A type 1-B/E, histone 113.1, histone 114, lamin A/C, lamina-associated polypeptide 2 isoform alpha, lamina-associated polypeptide 2 isoforms beta/gamma, lamin-B receptor, lamin-B1, lamin-B2, matrin-3, multiple inositol polyphosphate phosphatase 1, N-acylneuraminate cytidylyltransferase, neutral alpha-glucosidase AB, nuclear pore complex protein Nup93, nuclear pore membrane glycoprotein 210, nucleolin, nucleoporin NUP188 homolog, nucleoprotein TPR, prelamin-A/C, protein disulfide-isomerase, protein disulfide-isomerase A4, protein disulfide-isomerase A6, protein ERGIC-53, ribophorin II, transitional endoplasmic reticulum ATPase, UDP-glucose:glycoprotein glucosyltransferase 1, adeno-associated virus (AAV) capsid protein, alglocosidase alpha, anti-citrate synthase (anti-CS), anti-glycoprotein IIb/IIIa (anti-gp IIb/IIIa), anti-immunoglobulin E (anti-IGE), anti-interleukin-12 (anti-IL-12), anti-interleukin-23 (anti-IL-23), anti-RANK ligand, anti-alpha 4 integral, anti-a proliferation inducing ligand (anti-APRIL), anti-B cell activating factor of the TNF family (anti-BAFF), anti-cluster of differentiation 20 (anti-CD20), anti-cluster of differentiation 52 (CD52), anti-fibroblast growth factor receptor (anti-FGFR), anti-human epidermal growth factor receptor 2 (anti-Her2), anti-interleukin-12 (anti-IL2) receptor, anti-interleukin-6 (anti-IL6) receptor, anti-programmed cell death protein 1 (anti-PD1), anti-respiratory syncytial virus protein F (anti-RSV protein F), anti-tumor necrosis factor alpha (anti-TNF-alpha), anti-vascular endothelial growth factor (anti-VEGF), cytotoxic T-lymphocyte associated protein 4 (CTLA4), erythropoietin, factor IX, factor VII, factor VIII, glatiramer acetate, glucocerebrosidase, gonadotropin-releasing hormone (GnRH) antagonist, immunoglobulin G (IgG), interleukin-1 receptor (IL1R) antagonist, interleukin-1 (I) antagonist, insulin, interferon alpha, interferon beta, lentivirus capsid protein, lymphocyte function-associated antigen 3 fragment crystallizable region (LFA3-Fc), retrovirus capsid protein, transmembrane activator and cyclophilin ligand interactor immunoglobulin (TACI-Ig), and tumor necrosis factor (TNF) receptor.

27. The method of claim 6, wherein said pharmaceutical agent is selected from the group consisting of an ankyrin repeat protein, an antibody, an antibody-like binder, an aptamer, an ARM repeat protein, a carbohydrate, a complement-related protein, a cyclic peptide, a designed ankyrin repeat protein (DARPin), a DNAse, a fibronectin, a GPI-linked polypeptide, a HEAT repeat protein, a lipoprotein, a metal chelator, a nanobody, a nucleic acid, a polypeptide, a single-chain variable fragment (scFv), a tetratricopeptide repeat protein, a cell surface receptor, a complement receptor, and an enzyme.

28. The method of claim 27, wherein said complement-related protein is selected from the group consisting of a complement component 1 (C1) inhibitor, a complement component 4 (C4) binding protein, cluster of differentiation 59 (CD59), decay-accelerating factor (DAF), factor H, factor I, homologous restriction factor, membrane cofactor protein (MCP), and proline/arginine-rich end leucine-rich repeat protein (PRELP).

29. The method of claim 27, wherein said complement receptor is selected from the group consisting of C3 beta chain receptor, complement component 3a receptor (C3aR), complement receptor type 1 (CR1), complement receptor type 2 (CR2), complement receptor type 3 (CR3), and complement receptor type 4 (CR4).

30. The method of claim 27, wherein said enzyme is selected from the group consisting of triacylglycerol lipase, (S)-methylmalonyl-CoA hydrolase, acyl-carrier-protein phosphodiesterase, phosphorylase phosphatase, 1,4-lactonase, 11-cis-retinyl-palmitate hydrolase, 1-alkyl-2-acetylglycerophosphocholine esterase, 2′-hydroxybiphenyl-2-sulfinate desulfinase, 2-pyrone-4,6-dicarboxylate lactonase, 3′,5′-bisphosphate nucleotidase, 3-hydroxyisobutyryl-CoA hydrolase, 3′-nucleotidase, 3-oxoadipate enol-lactonase, 3-phytase, 4-hydroxybenzoyl-CoA thioesterase, 4-methyloxaloacetate esterase, 4-phytase, 4-pyridoxolactonase, 5′-nuclemidase, 6-acetylglucose deacetylase, 6-phosphogluconolactonase, alpha-amino-acid esterase, acetoacetyl-CoA hydrolase, acetoxybutynylbithiophene deacetylase, acetylajmaline esterase, acetylalkylglycerol acetylhydrolase, acetylcholinesterase, acetyl-CoA hydrolase, acetylesterase, acetylpyruvate hydrolase, acetylsalicylate deacetylase, acetylxylan esterase, acid phosphatase, actinomvcin lactonase, acylcarnitine hydrolase, acyl-CoA hydrolase, acylglycerol lipase, acyloxyacyl hydrolase, acylpyruvate hydrolase, a disintegrin and metalloproteinase with a thrombospondin type 1 motif member 13 (ADAMTS13), adenosine deaminase, adenylyl-[glutamate-ammonia ligase] hydrolase, ADP-dependent medium-chain-acyl-CoA hydrolase, ADP-dependent short-chain-acyl-CoA hydrolase, ADP-phosphoglycerate phosphatase, alkaline phosphatase, all-trans-retinyl-palmitate hydrolase, aminoacyl-tRNA hydrolase, arylesterase, arylsulfatase, asparaginase, bile-acid-CoA hydrolase, bis(2-ethylhexyl)phthalate esterase, bisphosphoglycerate phosphatase, carboxylesterase, carboxymethylenebutenolidase, cellulose-polysulfatase, cephalosporin-C deacetylase, cerebroside-sulfatase, cetraxate benzylesterase, chlorogenate hydrolase, chlorophyllase, cholinesterase, choline-sulfatase, choloyl-CoA hydrolase, chondro-4-stilfatase, chondro-6-sulfatase, citrate-lyase deacetylase, cocaine esterase, cutinase, cyclamate sulfohydrolase, D-arabinonolactonase, deoxylimonate A-ring-lactonase, dGTPase, dihydrocoumarin hydrolase, disulfoglucosamine-6-sulfatase, dodecanoyl-[acyl-carrier-protein] hydrolase, a factor IX, factor VIII, fatty-acyl-ethyl-ester synthase, beta-diketone hydrolase, feruloyl esterase, formyl-CoA hydrolase, fructose-bisphosphatase, fumarylacetoacetase, fusarinine-C ormthinesterase, galactolipase, gluconolactonase, glucose-1-phosphatase, glucose-6-phosphatase, glutathione thiolesterase, glycerol-1-phosphatase, glycerol-2-phosphatase, glycerophosphocholine phosphodiesterase, glycosulfatase, histidinol-phosphatase, hormone-sensitive lipase, hydroxyacylglutathione hydrolase, hydroxybutyrate-dimer hydrolase, hydroxymethylglutaryl-CoA hydrolase, iduronate-2-sulfatase, inositol-phosphate phosphatase, juvenile-hormone esterase, kynureninase, L-arabinonolactonase, limonin-D-ring-lactonase, lipoprotein lipase, L-rhamnono-1,4-lactonase, lysophospholipase, mannitol-1-phosphatase, methylphosphothioglycerate phosphatase, methylumbelliferyl-acetate deacetylase, monoterpene epsilon-lactone hydrolase, N-acetylgalactosamine-4-sulfatase, N-acetylgalactosamine-6-sulfatase, N-acetylgalactosaminoglycan deacetylase, N-acetylglucosiunine-6-sulfatase, N-sulfoglucosamme sulfohydrolase, oleoyl[acyl-carrier-protein] hydrolase, orsellinate-depside hydrolase, oxaloacetase, palmitoyl[protein] hydrolase, palmitoyl-CoA hydrolase, pectinesterase, phenylacetyl-CoA hydrolase, phenylalanine ammonia lyase, phenylalanine hydroxylase, pheophorbidase, phloretin hydrolase, phorbol-diester hydrolase, phosphatidate phosphatase, phosphatidylglycerophosphatase, phosphatidylinositol deacylase, phosphodiesterase I, phosphoglycerate phosphatase, phosphoglycolate phosphatase, phosphoinositide phospholipase C, phospholipase Al, phospholipase A2, phospholipase C, phospholipase D, phosphonoacetaldehyde hydrolase, phosphonoacetate hydrolase, phosphonopyruvate hydrolase, phosphoprotein phosphatase, phosphoserine phosphatase, poly(3-hydroxybutyrate) depolymemse, poly(3-hydroxyoctanoate) depolymerase, polyneuridine-aldehyde esterase, protein-glutamate methylesterase, quorum-quenching N-acyl-homoserine lactonase, retinyl-palmitate esterase, serine dehydratase, serine hydroxymethyl transferase, serine endopeptidase, serine-ethanolaminephosphate phosphodiesterase, S-formylglutathione hydrolase, sialate O-acetylesterase, sinapine esterase, sphingomyelin phosphodiesterase, S-succinylglutathione hydrolase, steroid-lactonase, sterol esterase, steryl-sulfatase, succinyl-CoA hydrolase, sucrose-phosphate phosphatase, sugar-phosphatase, tannase, thymidine phosphorylase, trehalose-phosphatase, triacetate-lactonase, trithionate hydrolase, tropinesterase, ubiquitin thiolesterase, UDP-sulfoquinovose synthase, uronolactonase, wax-ester hydrolase, xylono-1, 4-lactonase, 3-methylcrotonyl-CoA carboxylase, porphobilinogen deaminase, adenine phosphoribosyltransferase, adenosine deaminase, alcohol dehydrogenase, alcohol oxidase, homogentisate oxidase, ammonia monooxygenase, lecithin-cholesterol acyltransferase (LCAT), thiosulfate-cyanide sulfurtransferase, hexokinase, glucokinase, arginase, lysine oxidase, uricase, hepatic lipase (LIPC), lipoprotein lipase (LPL), cholesteryl ester transfer protein (CETP), N-aspartylglucosaminidase, sterol 27-hydroxylase, palmitoyl-protein thioesterase-1, lysosomal pepstatin-insensitive peptidase, lisosomal acid lipase, phosphomannomutase-2, mannose phosphate isomerase, dolichyl-P-glucose:Man-9-GlcNAc2-PP-dolichyl glucosyltransferase, dolichyl-P-mannose:Man-5-GlcNAc2-PP-dolichyl mannosyltransferase, dolichyl-P-mannose synthase, dolichyl-P-mannose:Man-7-GlcNAc2-PP-dolichyl-alpha-6-mannosyltransferase, dolichyl-P-glucose:Glc-1-Man-9-GlcNAc-2-PP-dolichyl-alpha-3-glucosyltransferase, alpha-1,3-mannosyltransferase, mannosyl-alpha-1,6-glycoprotein-beta-1,2-N-acetylglucosminyltransferase, glucosidase I, beta-1,4-galactosyltransferase, UDP-GlcNAc:dolichyl-P NAcGlc phosphotransferase, beta-1,4-mannosyltransferase, alpha-1,2-mannosyltransferase, trihexosylceramide alpha-galactosidase, ceramidase, alpha-L-fucosidase, glucosylcerainide beta-glucosidase, ADP-ribose protein hydrolase, alpha glucosidase, ganglioside beta-galactosidase, galactosylceramide beta-galactosidase, lysosomal acid lipase, arylsulfatase A, N-acetylglucosaminyl-1-phosphotransfeerase catalytic subunit, N-acetylglucosaminyl-1-phosphotransfeerase, N-acetylglucosaminyl-1-phosphotransfeerase substrate recognition subunit, alpha-1-iduronidase, iduronate sulfate sulfatase, heparan-S-sulfate sulfamidase, N-acetyl-D-glucosaminidase, acetyl-CoA-glucosaminide N-acetyltranaferase, N-acetyl-glucosaminine-6-sulfate sulfatase, galactosamine-6-sulfate sulfatase, beta-galactosidase, N-acetyl galactosamine alpha-4-sulfate sulfatase (arylsulfatase B), beta-glucuronidase, sphingomyelinase, beta-hexosaminidase B, N-acetyl-galactosaminidase, neuraminidase 1 (sialidase), UDP-N-acetylglucosamine-2-epimerase, N-acetylmannosamine kinase, ganglioside beta-galactosidase, beta-hexosaminidase A, metalloproteinase-2, lysosomal acid lipase, alpha-D-mannosidase, beta-D-mannosidase, methanol dehydrogenase, and butyrylcholinesterase.

31. The method of claim 27, wherein said antibody-like binder selected from the group consisting of an antibody-like binder to serum amyloid A protein or serum amyloid P component, an antibody-like binder to beta-2 microglobulin or serum amyloid P component, an antibody-like hinder to serum amyloid P component light chain, an antibody-like binder to cluster of differentiation 44 (CD44), an antibody-like binder to epithelial cellular adhesion molecule (EpCam), an antibody-like binder to human epidermal growth factor receptor 2 (Her2), an antibody-like binder to epidermal growth factor receptor (EGFR), an antibody-like binder to cluster of differentiation 20 (CD20), an antibody-like binder to cluster of differentiation 19 (CD19), an antibody-like binder to B. anthracis surface protein, an antibody-like binder to C. botulinum surface protein, an antibody-like binder to C. difficile surface protein, an antibody-like binder to candida surface protein, an antibody-like binder to E. coli surface protein, an antibody-like binder to Ebola surface protein, an antibody-like binder to hepatitis B virus(HBV) surface protein, an antibody-like binder to hepatitis C virus (HCV) surface protein, an antibody-like binder to human immunodeficiency virus (HIV) envelope proteins, an antibody-like binder to cluster of differentiation 4 (CD4), an antibody-like binder to a chemokine receptor (CCR), an antibody-like binder to M. tuberculosis surface protein, an antibody-like hinder to P. falriparutm surface protein, an antibody-like binder to prion protein PRP, an antibody-like binder to prion protein PRPc, an antibody-like binder to prion protein PRPsc, an antibody-like binder to prion protein PRPres, an antibody-like binder to Duffy antigen receptor of chemokines (DARC), an antibody-like binder to spider venom, an antibody-like binder to low-density lipoprotein (LDL) receptor, an antibody-like binder to high-density lipoprotein (HDL) receptor, an antibody-like binder to alpha hemolysin, an antibody-like binder to anthrax toxin, an antibody-like binder to bacterial toxin, and an antibody-like binder to botulinum toxin.

32. The method of claim 6, wherein said pharmaceutical agent is a protein selected from the group consisting of beta2-glycoprotein-I,I/i antigen, alpha-3 noncollagenous domain I (NCI) of collagen (IV), platelet glycoprotein Ib-IX, platelet glycoprotein IIb-IIIa, platelet glycoprotein IV, platelet glycoprotein Ia-IIa, phospholipase A2 receptor, glycophorin A, glycophorin B, glycophorin C, Rh antigen, complement factor H, complement factor I, factor I, factor II, factor XIII, factor XII, factor XI, factor V, factor X, transmembrane CLN3 protein, transmembrane CLN8 protein, transmembrane CLN6 protein, transmembrane CLN5 protein, mannose-P-dolichol utilization defect protein, GDP-fucose transporter-1, oligomeric Golgi complex-7, cystinosin, protective protein/cathepsin A (PPCA), GM2 activator protein, sialin, sulfatase-modifying factor-1, Niemann-Pick C1 (NPC1) protein, epididymal secretory protein 1, prosaposin, cathepsin K, saposin B, saposin C, and sialin.
